SEQUENCE LISTING (84 sequences)

SEQ ID NO: 1 - protein, 7 aa, Artificial Sequence
  Synthetic Construct
GGSFSGY

SEQ ID NO: 2 - protein, 5 aa, Artificial Sequence
  Synthetic Construct
NHSGS

SEQ ID NO: 3 - protein, 7 aa, Artificial Sequence
  Synthetic Construct
VINWFDP

SEQ ID NO: 4 - protein, 11 aa, Artificial Sequence
  Synthetic Construct
RASQDISSWLA

SEQ ID NO: 5 - protein, 7 aa, Artificial Sequence
  Synthetic Construct
AASSLQS

SEQ ID NO: 6 - protein, 9 aa, Artificial Sequence
  Synthetic Construct
QQANSFPYT

SEQ ID NO: 7 - protein, 112 aa, Artificial Sequence
  Synthetic Construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVT

SEQ ID NO: 8 - protein, 107 aa, Artificial Sequence [sequence omitted]

SEQ ID NO: 9 - protein, 8 aa, Artificial Sequence
  Synthetic Construct
GYTFTDYV

SEQ ID NO: 10 - protein, 8 aa, Artificial Sequence
  Synthetic Construct
TYTGSGSS

SEQ ID NO: 11 - protein, 9 aa, Artificial Sequence
  Synthetic Construct
RGKGTGFAF

SEQ ID NO: 12 - protein, 10 aa, Artificial Sequence
  Synthetic Construct
QSVDYDGDSY

SEQ ID NO: 13 - protein, 7 aa, Artificial Sequence
  Synthetic Construct
AASNLES

SEQ ID NO: 14 - protein, 9 aa, Artificial Sequence
  Synthetic Construct
QQSYEDPPT

SEQ ID NO: 15 - protein, 118 aa, Artificial Sequence
  Synthetic Construct
QVQLQQSGPELVKPGASVKMSCKASGYTFTDYVINWVKQRTGQGLEWIGETYTGSGSSYYNEKFKDKATLTVDKASNIAYMQLSSLTSEDSAVYFCARRGKGTGFAFWGQGTLVTVSA

SEQ ID NO: 16 - protein, 111 aa, Artificial Sequence
  Synthetic Construct
DIVLTQSPASLAVSLGQRATISCKASQSVDYDGDSYMNWYQQKPGQPPKLLIYAASNLESGIPARFTGSGSGTDFTLNIHPVEEEDTATYYCQQSYEDPPTFAGGTNLEIK

SEQ ID NO: 17 - protein, 7 aa, Artificial Sequence
  Synthetic Construct
GYTFTNY

SEQ ID NO: 18 - protein, 6 aa, Artificial Sequence
  Synthetic Construct
DPSTGY

SEQ ID NO: 19 - protein, 9 aa, Artificial Sequence
  Synthetic Construct
EGGIGGFAY

SEQ ID NO: 20 - protein, 15 aa, Artificial Sequence
  Synthetic Construct
RASESVDSYDNSFMH

SEQ ID NO: 21 - protein, 7 aa, Artificial Sequence
  Synthetic Construct
RASNLES

SEQ ID NO: 22 - protein, 9 aa, Artificial Sequence
  Synthetic Construct
QQSKEDPYT

SEQ ID NO: 23 - protein, 118 aa, Artificial Sequence
  Synthetic Construct
QVQLQQSGAELAKPGASVKMSCKASGYTFTNYLMHWVKQRPGQGLEWIGYIDPSTGYTVYLQKFKDKATLTADKSSSTTYMQLSSLTSEDSAVYYCAKEGGIGGFAYWGQGTLVTVSA

SEQ ID NO: 24 - protein, 111 aa, Artificial Sequence
  Synthetic Construct
DIVLTPSPASLAVSLGQRATISCRASESVDSYDNSFMHWYQQKPGQPPKLLIYRASNLESGIPARFSGSGSRTDFTLTIDPVEADDVATYYCQQSKEDPYTFGGGTKLEIK

SEQ ID NO: 25 - protein, 7 aa, Artificial Sequence
  Synthetic Construct
GYTFTSY

SEQ ID NO: 26 - protein, 6 aa, Artificial Sequence
  Synthetic Construct
NPYNDG

SEQ ID NO: 27 - protein, 13 aa, Artificial Sequence
  Synthetic Construct
EKDNYATGAWFAY

SEQ ID NO: 28 - protein, 13 aa, Artificial Sequence
  Synthetic Construct
KSSQSLLYSTNQK

SEQ ID NO: 29 - protein, 7 aa, Artificial Sequence
  Synthetic Construct
WASTRES

SEQ ID NO: 30 - protein, 8 aa, Artificial Sequence
  Synthetic Construct
QQYYSYRT

SEQ ID NO: 31 - protein, 123 aa, Artificial Sequence
  Synthetic Construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSA

SEQ ID NO: 32 - protein, 113 aa, Artificial Sequence
  Synthetic Construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKR

SEQ ID NO: 33 - protein, 478 aa, Artificial Sequence
  Synthetic Construct
MALPVTALLLPLALLLHAARPQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 488 aa, Artificial Sequence
  Synthetic Construct
MALPVTALLLPLALLLHAARPQVQLQQSGPELVKPGASVKMSCKASGYTFTDYVINWVKQRTGQGLEWIGETYTGSGSSYYNEKFKDKATLTVDKASNIAYMQLSSLTSEDSAVYFCARRGKGTGFAFWGQGTLVTVSAGGGGSGGGGSGGGGSDIVLTQSPASLAVSLGQRATISCKASQSVDYDGDSYMNWYQQKPGQPPKLLIYAASNLESGIPARFTGSGSGTDFTLNIHPVEEEDTATYYCQQSYEDPPTFAGGTNLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 488 aa, Artificial Sequence
  Synthetic Construct
MALPVTALLLPLALLLHAARPQVQLQQSGAELAKPGASVKMSCKASGYTFTNYLMHWVKQRPGQGLEWIGYIDPSTGYTVYLQKFKDKATLTADKSSSTTYMQLSSLTSEDSAVYYCAKEGGIGGFAYWGQGTLVTVSAGGGGSGGGGSGGGGSDIVLTPSPASLAVSLGQRATISCRASESVDSYDNSFMHWYQQKPGQPPKLLIYRASNLESGIPARFSGSGSRTDFTLTIDPVEADDVATYYCQQSKEDPYTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 495 aa, Artificial Sequence
  Synthetic Construct
MALPVTALLLPLALLLHAARPQVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSAGGGGSGGGGSGGGGSDIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 24 aa, Artificial Sequence
  Synthetic Construct
IYIWAPLAGTCGVLLLSLVITLYC

SEQ ID NO: 38 - protein, 42 aa, Artificial Sequence
  Synthetic Construct
KRGRKKLLYIFKQPFMRPVQTTQEEDGCSCRFPEEEEGGCEL

SEQ ID NO: 39 - protein, 112 aa, Artificial Sequence
  Synthetic Construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 45 aa, Artificial Sequence
  Synthetic Construct
TTTPAPRPPTPAPTIASQPLSLRPEACRPAAGGAVHTRGLDFACD

SEQ ID NO: 41 - protein, 26 aa, Artificial Sequence
  Synthetic Construct
VMSVATIVIVDICITGGLLLLVYYWS

SEQ ID NO: 42 - protein, 22 aa, Artificial Sequence
  Synthetic Construct
MQSGTHWRVLGLCLLSVGVWGQ

SEQ ID NO: 43 - protein, 104 aa, Artificial Sequence
  Synthetic Construct
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 44 - protein, 207 aa, Artificial Sequence
  Synthetic Construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 45 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 46 - protein, 7 aa, Artificial Sequence
  Synthetic Construct
GFSFSDC

SEQ ID NO: 47 - protein, 8 aa, Artificial Sequence
  Synthetic Construct
SVKSENYG

SEQ ID NO: 48 - protein, 13 aa, Artificial Sequence
  Synthetic Construct
SYYRYDVGAWFAY

SEQ ID NO: 49 - protein, 15 aa, Artificial Sequence
  Synthetic Construct
RASKSVSTSGYSYIY

SEQ ID NO: 50 - protein, 7 aa, Artificial Sequence
  Synthetic Construct
LASILES

SEQ ID NO: 51 - protein, 9 aa, Artificial Sequence
  Synthetic Construct
QHSRELPWT

SEQ ID NO: 52 - protein, 125 aa, Artificial Sequence
  Synthetic Construct
EEQLVESGGGLVKPGGSLRLSCAASGFSFSDCRMYWLRQAPGKGLEWIGVISVKSENYGANYAESVRGRFTISRDDSKNTVYLQMNSLKTEDTAVYYCSASYYRYDVGAWFAYWGQGTLVTVSSA

SEQ ID NO: 53 - protein, 112 aa, Artificial Sequence
  Synthetic Construct
DIVMTQSPDSLAVSLGERATINCRASKSVSTSGYSYIYWYQQKPGQPPKLLIYLASILESGVPDRFSGSGSGTDFTLTISSLQAEDVAVYYCQHSRELPWTFGQGTKVEIKR

SEQ ID NO: 54 - protein, 496 aa, Artificial Sequence
  Synthetic Construct
MALPVTALLLPLALLLHAARPEEQLVESGGGLVKPGGSLRLSCAASGFSFSDCRMYWLRQAPGKGLEWIGVISVKSENYGANYAESVRGRFTISRDDSKNTVYLQMNSLKTEDTAVYYCSASYYRYDVGAWFAYWGQGTLVTVSSAGGGGSGGGGSGGGGSDIVMTQSPDSLAVSLGERATINCRASKSVSTSGYSYIYWYQQKPGQPPKLLIYLASILESGVPDRFSGSGSGTDFTLTISSLQAEDVAVYYCQHSRELPWTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 7 aa, Artificial Sequence
  Synthetic Construct
GYTFTNY

SEQ ID NO: 56 - protein, 6 aa, Artificial Sequence
  Synthetic Construct
NTNTGE

SEQ ID NO: 57 - protein, 12 aa, Artificial Sequence
  Synthetic Construct
LGLYYDYGYYAM

SEQ ID NO: 58 - protein, 11 aa, Artificial Sequence
  Synthetic Construct
RASESVDSYGN

SEQ ID NO: 59 - protein, 7 aa, Artificial Sequence
  Synthetic Construct
LASNLES

SEQ ID NO: 60 - protein, 9 aa, Artificial Sequence
  Synthetic Construct
QQNNEDPYT

SEQ ID NO: 61 - protein, 123 aa, Artificial Sequence
  Synthetic Construct
QIQLVQSGPELKKPGETVKISCKASGYTFTNYGMNWVKQAPGKGLKCMGWINTNTGEPTYAEEFKGRFAFSLETSATTAFLQINNLKDEDTATYFCARLGLYYDYGYYAMDYWGQGASVTVSS

SEQ ID NO: 62 - protein, 111 aa, Artificial Sequence
  Synthetic Construct
NIVLTQSPASLAVSLGQRATISCRASESVDSYGNSFMHWYQQKPGQPPKLFIYLASNLESGVPARFSGSGSRTDFTLTIDPVEADDAATYYCQQNNEDPYTFGGGTKLEIK

SEQ ID NO: 63 - protein, 493 aa, Artificial Sequence
  Synthetic Construct
MALPVTALLLPLALLLHAARPQIQLVQSGPELKKPGETVKISCKASGYTFTNYGMNWVKQAPGKGLKCMGWINTNTGEPTYAEEFKGRFAFSLETSATTAFLQINNLKDEDTATYFCARLGLYYDYGYYAMDYWGQGASVTVSSGGGGSGGGGSGGGGSNIVLTQSPASLAVSLGQRATISCRASESVDSYGNSFMHWYQQKPGQPPKLFIYLASNLESGVPARFSGSGSRTDFTLTIDPVEADDAATYYCQQNNEDPYTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 456 aa, Artificial Sequence
  Synthetic Construct
MQSGTHWRVLGLCLLSVGVWGQQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARVINWFDPWGQGTLVTGGGGSGGGGSGGGGSDIQMTQSPSSVSASVGDRVTITCRASQDISSWLAWYQHKPGKAPKLLIYAASSLQSGVPSRFSGSGSGTDFTLTISSLQPEDFATYYCQQANSFPYTFGQGTKLEIKGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 65 - DNA, 10 bp, Artificial Sequence
  Synthetic Construct
  misc_feature 3, 4, 5, 6, 7, 8: n = A or T
ccnnnnnngg

SEQ ID NO: 66 - protein, 10 aa, Homo sapiens
GFAFSIYDMS

SEQ ID NO: 67 - protein, 11 aa, Artificial Sequence
  Synthetic Construct
RASQDISNYLN

SEQ ID NO: 68 - protein, 12 aa, Artificial Sequence
  Synthetic Construct
GDSVSSNSAAWN

SEQ ID NO: 69 - protein, 11 aa, Artificial Sequence
  Synthetic Construct
RASQTIWSYLN

SEQ ID NO: 70 - protein, 17 aa, Artificial Sequence
  Synthetic Construct
YISSGGGTTYYPDTVKG

SEQ ID NO: 71 - protein, 7 aa, Artificial Sequence
  Synthetic Construct
YTSILHS

SEQ ID NO: 72 - protein, 18 aa, Artificial Sequence
  Synthetic Construct
RTYYRSKWYNDYAVSVKS

SEQ ID NO: 73 - protein, 7 aa, Artificial Sequence
  Synthetic Construct
AASSLQS

SEQ ID NO: 74 - protein, 14 aa, Artificial Sequence
  Synthetic Construct
HSGYGSSYGVLFAY

SEQ ID NO: 75 - protein, 9 aa, Artificial Sequence
  Synthetic Construct
QQGNTLPWT

SEQ ID NO: 76 - protein, 12 aa, Artificial Sequence
  Synthetic Construct
EVTGDLEDAFDI

SEQ ID NO: 77 - protein, 9 aa, Artificial Sequence
  Synthetic Construct
QQSYSIPQT

SEQ ID NO: 78 - protein, 847 aa, Artificial Sequence
  Synthetic Construct
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 79 - protein, 123 aa, Artificial Sequence
  Synthetic Construct
EVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGSSYGVLFAYWGQGTLVTVSA

SEQ ID NO: 80 - protein, 107 aa, Artificial Sequence
  Synthetic Construct
DIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIK

SEQ ID NO: 81 - protein, 489 aa, Artificial Sequence
  Synthetic Construct
MALPVTALLLPLALLLHAARPEVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGSSYGVLFAYWGQGTLVTVSAGGGGSGGGGSGGGGSDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 124 aa, Artificial Sequence
  Synthetic Construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 83 - protein, 107 aa, Artificial Sequence
  Synthetic Construct
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 84 - protein, 490 aa, Artificial Sequence
  Synthetic Construct
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2022/0265711 A1
(43) Pub. Date: Aug. 25, 2022

(54) ENGINEERED IMMUNE CELLS COMPRISING A RECOGNITION MOLECULE

(71) Applicant: Nanjing Legend Biotech Co., Ltd., Nanjing, Jiangsu (CN)

(72) Inventors: Ming ZENG, Irving, TX (US); Lili CHEN, Huzhou, Zhejiang (CN); Xun LIU, Nanjing, Jiangsu (CN)

(21) Appl. No.: 17/611,542

(22) PCT Filed: May 15, 2020
(86) PCT No.: PCT/CN2020/090601

§ 371 Date: Nov. 15, 2021

(30) Foreign Application Priority Data
May 16, 2019 (CN) PCT/CN2019/087260

(51) Int. Cl. A61K 35/17; C12N 5/0783; C12N 15/62; C12N 15/86; C07K 14/725; A61P 35/00

(52) U.S. Cl. CPC A61K 35/17; C12N 5/0636; C12N 15/625; A61K 38/00; C07K 14/7051; A61P 35/00; C12N 2740/15042; C12N 15/86

(57) ABSTRACT

Provided is an engineered immune cell comprising on its surface a recognition molecule that comprises a binding moiety specifically binding to a target molecule on the surface of a target cell, wherein the target molecule comprises an extracellular domain, and wherein the immune cell is capable of killing a target cell that comprises on its surface the target molecule. In one aspect, the binding moiety specifically binds to a distal portion of the extracellular domain, and the immune cell is capable of killing a target cell that comprises on its surface both the target molecule and the recognition molecule. In another aspect, the binding moiety specifically binds to a proximal portion of the extracellular domain, and the engineered immune cell has no or reduced capability of killing a target cell comprising on its surface both the target molecule and the recognition molecule.

### CROSS-REFERENCE TO RELATED APPLICATIONS

This application claims priority benefit of International Patent Application No. PCT/CN2019/087260 filed May 16, 2019, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 761422003140.txt, date recorded: May 11, 2020, size: 78 KB).

### FIELD OF THE INVENTION

The invention relates to engineered immune cells (such as engineered T cells) comprising on their surface recognition molecules useful for treating infectious diseases and cancer.

### BACKGROUND OF THE INVENTION

T-cell mediated immunity is an adaptive process of developing antigen (Ag)-specific T lymphocytes to eliminate viruses, bacterial, parasitic infections or malignant cells.

CD4+ T cells play a most important coordinating role in the immune system, having a central role in both T cell mediated immunity and B cell mediated (or humoral) immunity. In T cell mediated immunity, CD4+ T cells play a role in the activation and maturation of CD8+ T cells. In B cell mediated immunity, CD4+ T cells are responsible for stimulating B cells to proliferate and to induce B cell antibody class switching.

The central role CD4+ T cells play is perhaps best illustrated by the aftermath of an infection with human immunodeficiency virus (HIV). The virus is a retrovirus, meaning it carries its genetic information as RNA along with a reverse transcriptase enzyme that allows for the production of DNA from its RNA genome once it has entered a host cell. The DNA can then be incorporated into affected host cells, at which point the viral genes are transcribed and more viral particles are produced and released by the infected cell.

HIV preferentially targets CD4+ T cells; as a result, an infected patient's immune system becomes increasingly compromised, as the population of the main coordinating cells of the immune system is decimated. In fact, the progression of HIV to acquired immunodeficiency syndrome (AIDS) is marked by the patient's CD4+ T cell count. This targeting of CD4+ T cells by the virus is also what results in the inability of infected patients to successfully mount productive immune responses against various pathogens, including opportunistic pathogens.

Targeting the virus with various pharmacological classes of drugs prevents viral resistance and has shown a significant efficacy in infected patients, but requires high levels of adherence by patients to ensure its complete efficacy. In fact, non-adherence can result in the emergence of drug-resistant strains, leading to further difficulties in effectively managing and treating both the disease and subsequent complications in patients.

Chimeric antigen receptors (CARs) are a class of synthetic receptors that reprogram lymphocyte specificity and function. Engineered T cells are applicable in principle to many types of cancer, pending further progress to identify suitable target antigens, overcome immunosuppressive tumor microenvironments, reduce toxicities, and prevent antigen escape. Advances in the selection of optimal T cells, genetic engineering, and cell manufacturing are poised to broaden applications of T-cell-based therapies and enable new applications in infectious diseases and autoimmunity. With the continuous development of CAR-T cell technology, researchers have simultaneously expressed some costimulatory molecules and antigen receptors that are closely related to T cell activation on the surface of T cells, enhancing the killing activity of T cells. Because the reprogramming method of CAR-T cells is to integrate the related gene sequences directly into the cell chromosome through lentivirus, the CAR-T cells can stably express the designed antigen receptors and costimulatory molecules for a long time. In theory, the functions of the antigen receptors and costimulatory molecule can be long-term and steady. A large number of clinical reports have shown that CAR-T cell therapies from autologous sources have promising effects on a variety of B-cell malignancies and multiple myeloma. However, some patients still relapse after receiving CAR-T cell therapies.

A typical CAR comprises an extracellular antigen recognition domain, a hinge domain, a transmembrane domain, an intracellular costimulatory domain and a TCR signaling motif. The antigen recognition domain could be a variable region from an antibody, or a natural receptor or ligand for the antigen. The antigen recognition domain plays a key role in CAR-T cell activation. The affinity of the antigen recognition domain may affect whether CAR-T cells can discriminate cells expressing high-level antigens from low-level antigens, which is critical when a CAR-T is designed to target tumor-associated antigens but not tumor-specific antigens. Tumor associated antigens are antigens expressed at high-level on tumor cells and at low-level on some normal cells. When no tumor-specific antigens are available to construct a CAR, affinity of CAR-T need to be fine-tuned in order to reduce off-tumor, on-target effects. A widely used antigen recognition domain is an scFv, which comprises a heavy chain variable domain and a light chain variable domain from an antibody. The scFvs recognize an epitope on the antigen. The epitope location may also play important roles in the functions of a CAR-T.

In a clinical report (NCT01626495), a patient relapsed 9 months after CD19-targeted CAR T cell (CTL019) infusion due to CD19⁻ leukemia that aberrantly expressed the anti-CD19 CAR. The CAR gene was unintentionally introduced into a single leukemic B cell during T cell manufacture, and its progeny cells bound in cis to the CD19 epitope on the surface of leukemic cells, masking such cells from recognition by CTLO19 and leading to resistance to CTL019.

The disclosures of all publications, patents, patent applications and published patent applications referred to herein are hereby incorporated herein by reference in their entirety.

### BRIEF SUMMARY OF THE INVENTION

The present application in one aspect provides recognition molecules (e.g., transmembrane receptor), engineered immune cells, compositions and methods of treatment.

One aspect of the present application provides an engineered immune cell (“anti-distal portion engineered immune cell”) comprising on its surface a recognition molecule that comprises a binding moiety specifically binding to a target molecule on the surface of a target cell, wherein the target molecule comprises an extracellular domain, wherein the binding moiety specifically binds to a distal portion of the extracellular domain, wherein the immune cell is capable of killing a target cell that comprises on its surface the target molecule, and wherein the immune cell is capable of killing a target cell that comprises on its surface both the target molecule and the recognition molecule. In some embodiments, the recognition molecule comprises the binding moiety, a transmembrane domain, and an intracellular signaling domain. In some embodiments, the binding moiety is a single domain antibody (sdAb), an scFv, a Fab′, a (Fab′)₂, an Fv, or a peptide ligand.

In some embodiments according to one or more of the above embodiments of the anti-distal portion engineered immune cells, the distance from the distal portion of the extracellular domain to the membrane of the target cell is more than about 0.5 times of the distance from the binding moiety to the membrane of engineered immune cell. In some embodiments, the distance from the distal portion of the extracellular domain to the membrane of the target cell is more than about 1 time of the distance from the binding moiety to the membrane of engineered immune cell. In some embodiments, the distance from the distal portion of the extracellular domain to the membrane of the target cell is more than about 1.5 times of the distance from the binding moiety to the membrane of engineered immune cell. In some embodiments, the distance from the distal portion of the extracellular domain to the membrane of the target cell is more than about 2 times of the distance from the binding moiety to the membrane of engineered immune cell.

In some embodiments according to one or more of the above embodiments of the anti-distal portion engineered immune cells, the extracellular domain of the target molecule is at least about 175 amino acids long. In some embodiments, the binding moiety binds to a region in the extracellular domain that is about 50 amino acids or more away from the C-terminus of the extracellular domain. In some embodiments, the binding moiety binds to a region in the extracellular domain that is about 80 amino acids or more away from the C-terminus of the extracellular domain. In some embodiments, the binding moiety binds to a region that is within about 120 amino acids from the N-terminus of the extracellular domain. In some embodiments, the binding moiety binds to a region that is within about 80 amino acids from the N-terminus of the extracellular domain.

In some embodiments according to one or more of the above embodiments of the anti-distal portion engineered immune cells, the distal portion of the extracellular domain is at least about 30 Å away from the membrane of the target cell. In some embodiments, the distal portion of the extracellular domain is at least about 40 Å away from the membrane of the target cell. In some embodiments, the distal portion of the extracellular domain is at least about 60 Å away from the membrane of the target cell. In some embodiments, the distal portion of the extracellular domain is at least about 90 Å away from the membrane of the target cell. In some embodiments, the distal portion of the extracellular domain is at least about 120 Å away from the membrane of the target cell.

In some embodiments according to one or more of the above embodiments of the anti-distal portion engineered immune cells, the extracellular domain of the target molecule comprises three or more Ig-like domains. In some embodiments, the binding moiety binds to a region outside the first two Ig-like domains from the C-terminal end of the extracellular domain. In some embodiments, the binding moiety binds to a region outside the first four Ig-like domains from the C-terminal end of the extracellular domain. In some embodiments, the binding moiety binds to a region within the first three (e.g., within the first) Ig-like domain at the N-terminal end of the extracellular domain. In some embodiments, the binding moiety binds to a region within the first Ig-like domain at the N-terminal end of the extracellular domain.

In some embodiments according to one or more of the above embodiments of the anti-distal portion engineered immune cells, the target molecule is a transmembrane receptor. In some embodiments, the target molecule is selected from the group consisting of CD22, CD4, CD21 (CR2), CD30, ROR1, CD5, and CD20.

In some embodiments according to one or more of the above embodiments of the anti-distal portion engineered immune cells, the target molecule is CD22. In some embodiments, the binding moiety competes for binding with a reference antibody that specifically binds to an epitope within Domains 1-4 of CD22 (“anti-CD22 D1-4 antibody”). In some embodiments, the binding moiety binds to an epitope in Domains 1-4 of CD22 that overlaps with the binding epitope of a reference anti-CD22 D1-4 antibody. In some embodiments, the binding moiety comprises the same heavy chain and light chain CDR sequences as those of a reference anti-CD22 D1-4 antibody. In some embodiments, the binding moiety comprises the same heavy chain variable domain (VH) and light chain variable domain (VL) sequences as those of a reference anti-CD22 D1-4 antibody. In some embodiments, the reference anti-CD22 D1-4 antibody comprises a heavy chain CDR1 (HC-CDR1) comprising the amino acid sequence of SEQ ID NO: 67, a heavy chain CDR2 (HC-CDR2) comprising the amino acid sequence of SEQ ID NO: 68, a heavy chain CDR3 (HC-CDR3) comprising the amino acid sequence of SEQ ID NO: 69, a light chain CDR1 (LC-CDR1) comprising the amino acid sequence of SEQ ID NO: 70, a light chain CDR2 (LC-CDR2) comprising the amino acid sequence of SEQ ID NO: 71, and a light chain CDR3 (LC-CDR3) comprising the amino acid sequence of SEQ ID NO: 72. In some embodiments, the reference anti-CD22 D1-4 antibody comprises a VH comprising the amino acid sequence of SEQ ID NO: 73 and a VL comprising the amino acid sequence of SEQ ID NO: 74.

In some embodiments according to one or more of the above embodiments of the anti-distal portion engineered immune cells, the engineered immune cell is capable of killing a target cell that comprises on its surface both the target molecule and the recognition molecule by at least 3 fold as compared to an engineered immune cell comprising on its surface a recognition molecule comprising a binding moiety that binds to a proximal portion of the extracellular domain of the target molecule.

One aspect of the present application provides an engineered immune cell (“anti-proximal portion engineered immune cell”) comprising on its surface a recognition molecule that comprises a binding moiety specifically binding to a target molecule on the surface of a target cell, wherein the target molecule comprises an extracellular domain, wherein the binding moiety specifically binds to a proximal portion of the extracellular domain, wherein the engineered immune cell is capable of killing a target cell that comprises on its surface the target molecule, and wherein the engineered immune cell has no or reduced capability of killing a target cell comprising on its surface both the target molecule and the recognition molecule. In some embodiments, the recognition molecule comprises the binding moiety, a transmembrane domain, and an intracellular signaling domain. In some embodiments, the binding moiety is an sdAb, an scFv, a Fab′, a (Fab′)2, an Fv, or a peptide ligand.

In some embodiments according to one or more of the above embodiments of the anti-proximal portion engineered immune cells, the distance from the proximal portion of the extracellular domain to the membrane of the target cell is no more than about 2 times of the distance from the binding moiety to the membrane of engineered immune cell. In some embodiments, the distance from the proximal portion of the extracellular domain to the membrane of the target cell is no more than about 1.5 times of the distance from the binding moiety to the membrane of engineered immune cell. In some embodiments, the distance from the proximal portion of the extracellular domain to the membrane of the target cell is no more than about 1 time of the distance from the binding moiety to the membrane of engineered immune cell.

In some embodiments according to one or more of the above embodiments of the anti-proximal portion engineered immune cells, the extracellular domain of the target molecule is at least about 175 amino acids long. In some embodiments, the binding moiety binds outside of a region that is about 80 amino acids or more away from the N-terminus of the extracellular domain. In some embodiments, the binding moiety binds to a region in the extracellular domain that is within about 120 amino acids from the C-terminus of the extracellular domain. In some embodiments, the binding moiety binds to a region in the extracellular domain that is within about 102 amino acids from the C-terminus of the extracellular domain. In some embodiments, the binding moiety binds to a region in the extracellular domain that is within about 50 amino acids from the C-terminus of the extracellular domain.

In some embodiments according to one or more of the above embodiments of the anti-proximal portion engineered immune cells, the proximal portion of the extracellular domain is no more than about 120 Å away from the membrane of the target cell. In some embodiments, the proximal portion of the extracellular domain is no more than about 90 Å away from the membrane of the target cell. In some embodiments, the proximal portion of the extracellular domain is no more than about 60 Å away from the membrane of the target cell.

In some embodiments according to one or more of the above embodiments of the anti-proximal portion engineered immune cells, the extracellular domain of the target molecule comprises two or more Ig-like domains. In some embodiments, the binding moiety binds to a region outside the first Ig-like domain at the N-terminal end of the extracellular domain. In some embodiments, the binding moiety binds to a region outside the first three Ig-like domain at the N-terminal end of the extracellular domain. In some embodiments, the binding moiety binds to a region within the first four Ig-like domains from the C-terminal end of the extracellular domain. In some embodiments, the binding moiety binds to a region within the first two Ig-like domains from the C-terminal end of the extracellular domain.

In some embodiments according to one or more of the above embodiments of the anti-proximal portion engineered immune cells, the target molecule is a transmembrane receptor. In some embodiments, the target molecule is selected from the group consisting of CD22, CD4, CD21 (CR2), CD30, ROR1, CD5, and CD20.

In some embodiments according to one or more of the above embodiments of the anti-proximal portion engineered immune cells, the target molecule is CD22. In some embodiments, the binding moiety competes for binding with a reference antibody that specifically binds to an epitope within Domains 5-7 of CD22 (“anti-CD22 D5-7 antibody”). In some embodiments, the binding moiety binds to an epitope in Domains 5-7 of CD22 that overlaps with the binding epitope of a reference anti-CD22 D5-7 antibody. In some embodiments, the binding moiety comprises the same heavy chain and light chain CDR sequences as those of a reference anti-CD22 D5-7 antibody. In some embodiments, the binding moiety comprises the same VH and VL sequences as those of a reference anti-CD22 D5-7 antibody. In some embodiments, the reference anti-CD22 D5-7 antibody comprises a HC-CDR1 comprising the amino acid sequence of SEQ ID NO: 76, a HC-CDR2 comprising the amino acid sequence of SEQ ID NO: 77, a HC-CDR3 comprising the amino acid sequence of SEQ ID NO: 78, a LC-CDR1 comprising the amino acid sequence of SEQ ID NO: 79, a LC-CDR2 comprising the amino acid sequence of SEQ ID NO: 80, and a LC-CDR3 comprising the amino acid sequence of SEQ ID NO: 81. In some embodiments, the reference anti-CD22 D5-7 antibody comprises a VH comprising the amino acid sequence of SEQ ID NO: 82 and a VL comprising the amino acid sequence of SEQ ID NO: 83.

In some embodiments according to one or more of the above embodiments of the anti-proximal portion engineered immune cells, the engineered immune cell kills a target cell that comprises on its surface both the target molecule and the recognition molecule by no more than about 20% as compared to an engineered immune cell comprising on its surface a recognition molecule comprising a binding moiety that binds to a distal end of the extracellular domain of the target molecule.

In some embodiments according to one or more of the above embodiments of the engineered immune cells (including anti-distal portion and anti-proximal portion engineered immune cells), the recognition molecule is monospecific. In some embodiments, the recognition molecule is multispecific. In some embodiments, the recognition molecule comprises a second binding moiety specifically recognizing a second target molecule. In some embodiments, the second binding moiety is an sdAb, an scFv, a Fab′, a (Fab′)₂, an Fv, or a peptide ligand. In some embodiments, the binding moiety and the second binding moiety are linked in tandem. In some embodiments, the binding moiety is N-terminal to the second binding moiety. In some embodiments, the binding moiety is C-terminal to the second antigen binding moiety. In some embodiments, the binding moiety and the second binding moiety are linked via a linker.

In some embodiments according to one or more of the above embodiments of the engineered immune cells, the binding moiety is fused to the transmembrane domain directly or indirectly. In some embodiments, the binding moiety is non-covalently bound to a polypeptide comprising the transmembrane domain. In some embodiments, the recognition molecule comprises i) a first polypeptide comprising the binding moiety and a first member of a binding pair; and ii) a second polypeptide comprising a second member of the binding pair, wherein the first member and the second member bind to each other, and wherein the second member is fused to the transmembrane domain directly or indirectly. In some embodiments, the binding moiety is fused to a polypeptide comprising the transmembrane domain.

In some embodiments according to one or more of the above embodiments of the engineered immune cells, the recognition molecule is a chimeric antigen receptor (“CAR”). In some embodiments, the transmembrane domain is derived from a molecule selected from the group consisting of CD8α, CD4, CD28, 4-1BB, CD80, CD86, CD152 and PD1. In some embodiments, the transmembrane domain is derived from CD8α. In some embodiments, the intracellular signaling domain comprises a primary intracellular signaling domain derived from CD3ζ, FcRγ, FcRβ, CD3γ, CD3δ, CD3ε, CD5, CD22, CD79a, CD79b, or CD66d. In some embodiments, the primary intracellular signaling domain is derived from CD3ζ. In some embodiments, the intracellular signaling domain comprises a co-stimulatory signaling domain. In some embodiments, the co-stimulatory signaling domain is derived from a co-stimulatory molecule selected from the group consisting of CD27, CD28, 4-1BB, OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, DAP10, DAP12, CD83, ligands of CD83 and combinations thereof. In some embodiments, the co-stimulatory signaling domain comprises a cytoplasmic domain of 4-1BB. In some embodiments, the recognition molecule further comprises a hinge domain located between the C-terminus of the binding moiety and the N-terminus of the transmembrane domain. In some embodiments, the hinge domain is derived from CD8a or IgG4 CH2-CH3.

In some embodiments according to one or more of the above embodiments of the engineered immune cells, the recognition molecule is a chimeric T cell receptor (“cTCR”). In some embodiments, the transmembrane domain is derived from the transmembrane domain of a TCR subunit selected from the group consisting of TCRα, TCRβ, TCRγ, TCRδ, CD3γ, CD3ε, and CD3δ. In some embodiments, the transmembrane domain is derived from the transmembrane domain of CD3. In some embodiments, the intracellular signaling domain is derived from the intracellular signaling domain of a TCR subunit selected from the group consisting of TCRα, TCRβ, TCRγ, TCRδ, CD3γ, CD3ε, and CD3δ. In some embodiments, the intracellular signaling domain is derived from the intracellular signaling domain of CD3. In some embodiments, the transmembrane domain and intracellular signaling domain of the recognition molecule are derived from the same TCR subunit. In some embodiments, the recognition molecule further comprises at least a portion of an extracellular domain of a TCR subunit. In some embodiments, the binding moiety is fused to the N-terminus of CD3F (“eTCR”).

In some embodiments according to one or more of the above embodiments of the engineered immune cells, the engineered immune cell is a T cell. In some embodiments, the immune cell is selected from the group consisting of a cytotoxic T cell, a helper T cell, a natural killer (NK) cell, a natural killer T (NK-T) cell, and a γδT cell. In some embodiments, the engineered immune cell further comprises a co-receptor. In some embodiments, the co-receptor is a chemokine receptor.

In some embodiments according to one or more of the above embodiments of the engineered immune cells, the target cell is an immune cell. In some embodiments, the target cell is a tumor cell.

One aspect of the present application provides a pharmaceutical composition (“anti-distal portion pharmaceutical composition”) comprising the engineered immune cell according to any one of the anti-distal portion engineered immune cells described above.

One aspect of the present application provides a method of treating an individual having a cancer, comprising administering to the individual an effective amount of the pharmaceutical composition according to any one of the anti-distal portion pharmaceutical compositions described above. In some embodiments, the engineered immune cells are autologous to the individual. In some embodiments, the cancer is selected from the group consisting of T cell lymphoma, leukemia, B-cell precursor acute lymphoblastic leukemia (ALL), and B-cell lymphoma.

One aspect of the present application provides a method of treating an individual having an infectious disease, comprising administering to the individual an effective amount of the pharmaceutical composition according to any one of the anti-distal portion pharmaceutical compositions described above. In some embodiments, the engineered immune cells are autologous to the individual. In some embodiments, the infectious disease is an infection by a virus selected from the group consisting of HIV and HTLV. In some embodiments, the infectious disease is HIV.

One aspect of the present application provides a pharmaceutical composition comprising the anti-proximal portion engineered immune cell according to any one of the anti-proximal portion engineered immune cell embodiments described above.

One aspect of the present application provides a method of treating an individual having a cancer, comprising administering to the individual an effective amount of the pharmaceutical composition according to any one of the anti-proximal portion pharmaceutical compositions described above. In some embodiments, the engineered immune cells are allogeneic to the individual. In some embodiments, the cancer is selected from the group consisting of T cell lymphoma, leukemia, B-cell precursor acute lymphoblastic leukemia (ALL), and B-cell lymphoma.

One aspect of the present application provides a method of treating an individual having an infectious disease, comprising administering to the individual an effective amount of the pharmaceutical composition according to any one of the anti-proximal portion pharmaceutical compositions described above. In some embodiments, the engineered immune cells are allogeneic to the individual. In some embodiments, the infectious disease is an infection by a virus selected from the group consisting of HIV and HTLV. In some embodiments, the infectious disease is HIV.

Another aspect of the present application provides a method of making the engineered immune cell according to any one of the anti-distal or anti-proximal portion engineered immune cells described above, comprising introducing one or more nucleic acids encoding the recognition molecule into an immune cell, thereby obtaining the engineered immune cell.

Also provided are distal portion recognition molecules (e.g., transmembrane receptors), anti-distal portion engineered immune cells, or compositions according to any one of the embodiments described above for use in treating a cancer or an infectious disease (e.g., HIV), and use of proximal portion recognition molecules (e.g., transmembrane receptors), anti-proximal engineered immune cells, or compositions according to any one of the embodiments described above for use in treating a cancer or an infectious disease (e.g., HIV).

Further provided are compositions, kits and articles of manufacture comprising any one of the engineered immune cells described above.

### BRIEF DESCRIPTION OF THE DRAWINGS

FIG. 1A shows the structure of an exemplary anti-CD4 CAR, which is composed of a CD4 binding moiety, a hinge region, a transmembrane domain, a co-stimulatory domain and a CD3ζ signaling domain. The CD4 binding moiety can specifically recognize an epitope in Domain 1 of CD4 or an epitope in Domain 2 and/or 3 of CD4.

FIG. 1B shows phenotypes of two different kinds of anti-CD4 CAR-T cells. The CAR in CAR-T No. 1 contains an scFv specifically recognizing an epitope in Domain 1 of CD4, and can kill the CD4+ cells in both CAR+ and CAR− population. The CAR in CAR-T No. 2 contains an scFv specifically recognizing an epitope in domain 2 of CD4 and was not effective in killing the CAR+ target CD4+ cells.

FIG. 2 shows domain mapping of anti-CD4 antibodies Ibalizumab, Tregalizumab, and Zanolimumab. Mouse CD4 substituted with five different domains of human CD4 were transiently expressed on HEK-293 T cells. The antibodies were used to detect these domains by flow cytometry. The Zanolimumab VH/VL was used to generate CAR-T No. 1, and Ibalizumab VH/VL was used to generate CAR-T No. 2. Tregalizumab VH/VL was used to generate CAR-T No. 3.

FIGS. 3A and 3B show a hypothetical CAR-T and CD4 interaction model. FIG. 3A shows that CAR-T No. 1 recognizes an epitope in CD4 Domain 1, and CAR-T No. 2 recognizes an epitope in CD4 Domain 2 or 3. FIG. 3B shows that CD4 on CAR-T No. 2 is blocked in-cis by the CAR on the same cell, while CD4 on CAR-T No. 1 is not blocked and can be recognized by another CAR-T cell.

FIGS. 4A-4C show results of antibody blocking assays. FIG. 4A shows epitope binning for Ibalizumab, Tregalizumab, and Zanolimumab. FIG. 4B shows flow cytometry of CAR-T cells co-cultured with CSFE labeled pan T target cells in the absence or presence of different anti-CD4 antibodies. Two blocking doses were used, at 50 nM and 100 nM, respectively. FIG. 4C shows quantitative analysis of the CAR-T cells in FIG. 4B.

FIG. 5 shows the cytotoxic effects of anti-CD4 CAR-T cells. Two types of antibodies recognizing CD4 Domain 1 were used in the CAR-T cells of this experiment. UNT cells (un-transduced T cells) and CAR-T cells were co-cultured with CFSE labeled pan T target cells at E:T (effector: target) ratio of 0.5:1 for 24 hours. The expression of CD4 was detected by flow cytometry.

FIG. 6A shows flow cytometry results of human cutaneous T lymphoma cell line HH transduced with CARs. CAR % rate was detected by flow cytometry. Untransduced HH cells were used as control. FIG. 6B shows flow cytometry results of CFSE labeled HH or CAR-HH cells co-cultured with effector cells. CD4 Domain 1 specific CAR-T cells were used as effector cells. CAR-T No. 1 and UNT cells were used as control. CD4 expression on target cells was detected by flow cytometry. FIG. 6C shows relative CD4+% in each sample calculated based on UNT+HH sample. FIG. 6D shows effects of CAR-T NO. 1 cells on tumor growth (top) and body weight (bottom).

FIG. 7 shows the in vivo efficacy of anti-CD4 Domain 1 CAR-T No. 1 cells. Mice with human immune system (HIS mice) were inoculated with 3×10⁵ CAR+ CAR-T cells or UNT control cells. Splenocytes were harvested for flow cytometry analysis on day 18 post adoptive T cell treatment.

FIGS. 8A-8D show characterization of anti-CD4 Domain 1 eTCR-T cells. FIG. 8A shows percentages of TCR+ T cells in the anti-CD4 eTCR transduced T cell population. FIG. 8B shows IFNγ production by the anti-CD4 eTCR-T cells. FIG. 8C shows expansion of anti-CD4 eTCR-T cells. FIG. 8D shows in vitro killing effects of anti-CD4 eTCR-T cells against target cells. The sequence of this an anti-CD4 eTCR is listed in SEQ ID NO: 64.

FIG. 9 shows cytotoxic effects of anti-CD4 CAR-T cells. Two types of antibodies recognizing CD4 Domain 2 and/or Domain 3 were used in the CAR-T cells of this experiment. UNT cells (un-transduced T cells) and CAR-T cells were co-cultured with CFSE labeled pan-T target cells at E:T (effector: target) ratio of 0.5:1 for 24 hours. Expression of CD4 was detected by flow cytometry.

FIG. 10 shows the structure of an exemplary anti-CD22 CAR, which comprises a CD22 binding moiety, a hinge region, a transmembrane domain, a co-stimulatory domain and a CD3ζ signaling domain. The CD22 binding moiety can specifically recognize an epitope in Domains 1-4 of CD22 or an epitope in Domains 5-7 of CD22.

FIG. 11A shows CD22 domains recognized by the two anti-CD22 CARs used in the experiment. FIG. 11B shows cytotoxic effects of CAR-T No. 454, which recognizes Domain 3 of CD22. FIG. 11C shows cytotoxic effects of CAR-T No. 447, which recognizes Domains 5-7 of CD22. UNT cells (un-transduced T cells) and CAR-T cells were co-cultured with CFSE-labeled pan T target cells at E:T (effector:target) ratio of 0.5:1 for 24 hours. Expression of CD22 was detecting by flow cytometry.

FIG. 12 shows structures of the extracellular domains of CD22 and CD4.

### DETAILED DESCRIPTION OF THE INVENTION

The present application provides engineered immune cells comprising on its surface a recognition molecule that binds to an epitope within a specific region of a corresponding target molecule on the surface of a target cell. Exemplary recognition molecules include chimeric antigen receptors (“CARs”), chimeric T cell receptors (“cTCRs”), and other receptors that function within immune cells. The present application is based on the surprising discovery that certain types of recognition molecules, when expressed on the surface of an immune cell, can lead to depletion or elimination of the engineered immune cells, e.g., by other immune cells expressing the same recognition molecule (referred to as “self-killing capability”). Other types of recognition molecules, on the other hand, do not have such self-killing capability and instead can protect the immune cell from being killed by other immune cells expressing the same recognition molecule. It was discovered that the type of recognition molecules having self-killing capability contain a binding moiety that specifically recognizes a distal portion of the target molecule (i.e., a portion that is away from the cell membrane), while those that do not have such self-killing capability contain a binding moiety that specifically recognize a proximal portion (i.e., a portion that is close to the cell membrane) of the target molecule.

The present application demonstrates this principle with two exemplary target molecules, namely, CD4 and CD22. For example, we describe CAR-T cells that specifically recognize and respond to CD4+ cells or CD22+ cells. We discovered that anti-CD4 Domain 1 CAR-T not only kills CD4+ cells in the CAR negative cell population, but also eliminates CD4+CAR+ cells. In contrast, anti-CD4 Domains 2/3 CAR-T cannot eliminate CD4+CAR+ cells. Similarly, anti-CD22 Domains 1-4 CAR-T not only kills CD22+ cells in the CAR negative cell population, but also eliminates CD22+CAR+ cells. In contrast, CAR-T recognizing Domains 5-7 of CD22 could not eliminate CD22+CAR+ cells.

Without being bound by theory, it is hypothesized that binding molecules on the surface of an engineered immune cell differ in their self-killing capability depending on the epitope its binding moiety recognizes. A binding moiety recognizing a proximal end of a target molecule may be within a proper distance from an endogenously expressed target molecule on the same cell to block recognition of the epitope by another engineered immune cell, thus protecting the engineered immune cell from being attacked. A binding moiety recognizing a distal end of a target molecule, on the other hand, may be too far away from endogenously expressed target molecule on the same cell to block recognition of the target molecule by another engineered immune cell, thus leading to killing of the engineered immune cell.

So far, most engineered immune cells (such as CAR-T cells) are manufactured from autologous immune cells enriched from the individual to be treated. For HIV treatment, if the original immune cells contain the HIV virus, the engineered immune cells may also contain the HIV virus and become the source of new infection. For example, for treating CD4+ T cell lymphoma/leukemia with engineered immune cells (such as CAR-T), any CD4+ leukemia/lymphoma cell contaminated in the immune cell population will need to be removed. During engineered immune cell manufacturing, residual tumor cells in the enriched T cell population could also be transduced with the lentivirus expressing the immune cell receptor and become positive for the immune cell receptor. An immune cell receptor can bind to its ligand in-cis, thus masking the targeting antigen on the engineered immune cells. The tumor cells expressing the immune cell receptor then can escape the immune cell receptor mediated killing and eventually lead to resistant disease relapse. The distal end-binding molecules described herein, which possess the ability of self-killing, would thus be particularly suitable for autologous treatment methods.

In contrast, the risk of autologous immune cells discussed above does not exist in the context of allogeneic treatment. In the allogeneic context, it is desirable that the engineered immune cells do not kill themselves, so that the efficacy of the engineered immune cells can be realized to their maximum. The proximal end-binding molecules described herein, which do not possess the ability of self-killing, would thus be particularly suitable for allogeneic treatment methods.

Thus, the present application in one aspect provides an engineered immune cell comprising on its surface a recognition molecule that comprises a binding moiety specifically binding to a target molecule on the surface of a target cell, wherein the target molecule comprises an extracellular domain, wherein the binding moiety specifically binds to a distal portion of the extracellular domain, wherein the immune cell is capable of killing a target cell that comprises on its surface the target molecule, and wherein the immune cell is capable of killing a target cell that comprises on its surface both the target molecule and the recognition molecule. These engineered immune cells (“anti-distal portion engineered immune cells”) are particularly useful for autologous treatment of diseases, such as cancer and infectious diseases.

In another aspect, the present application provides an engineered immune cell comprising on its surface a recognition molecule that comprises a binding moiety specifically binding to a target molecule on the surface of a target cell, wherein the target molecule comprises an extracellular domain, wherein the binding moiety specifically binds to a proximal portion of the extracellular domain, wherein the engineered immune cell is capable of killing a target cell that comprises on its surface the target molecule, and wherein the engineered immune cell has no or reduced capability of killing a target cell comprising on its surface both the target molecule and the recognition molecule. These engineered immune cells (“anti-proximal portion engineered immune cells”) are particularly useful for allogeneic treatment of diseases, such as cancer and infectious diseases.

### Definitions

The term “distal portion” used herein refers to an extracellular region in a target molecule on the surface of a cell that is away from the cell membrane relative to other extracellular regions in the target cell.

The term “proximal portion” used herein refers to an extracellular region in a target molecule on the surface of a cell that is close to the cell membrane relative to other extracellular regions in the target cell.

As used herein, the “distance” from a region of a molecule (e.g., the distal portion or proximal portion of an extracellular domain of a target molecule, or the binding moiety of the recognition molecule) to the membrane of the a cell that expresses the molecule refers to the distance from the center of mass among amino acid residues in the region that are involved in binding with its binding partner (e.g., the binding moiety of the recognition molecule or the distal portion or proximal portion of an extracellular domain of a target molecule, respectively) to the membrane of the cell.

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The term antibody includes conventional four-chain antibodies, and single-domain antibodies, such as heavy-chain only antibodies or fragments thereof, e.g., VHH.

A full-length four-chain antibody comprises two heavy chains and two light chains. The variable regions of the light and heavy chains are responsible for antigen binding. The variable domains of the heavy chain and light chain may be referred to as “V_{H}” and “V_{L}”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani, 1997, J. Mol. Biol., 273:927-948; Chothia 1985, J. Mol Biol., 186: 651-663; Chothia 1987, J. Mol. Biol., 196: 901-917; Chothia 1989, Nature, 342:877-883; Kabat 1987, Sequences of Proteins of Immunological Interest, Fourth Edition. US Govt. Printing Off. No. 165-492; Kabat 1991, Sequences of Proteins of Immunological Interest, Fifth Edition. NIH Publication No. 91-3242). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as lgG1 (γ1 heavy chain), lgG2 (γ2 heavy chain), lgG3 (γ3 heavy chain), lgG4 (γ4 heavy chain), lgA1 (α1 heavy chain), or lgA2 (α2 heavy chain).

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, single-domain antibodies are engineered from camelid HCAbs, and their heavy chain variable domains are referred herein as “VHHs” (Variable domain of the heavy chain of the Heavy chain antibody). Camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)). A basic VHH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

The term “antibody moiety” includes full-length antibodies and antigen-binding fragments thereof. A full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and heavy chains, respectively. Several of the major antibody classes are divided into subclasses such as lgG1 (γ1 heavy chain), lgG2 (γ2 heavy chain), lgG3 (γ3 heavy chain), lgG4 (γ4 heavy chain), lgA1 (al heavy chain), or lgA2 (α2 heavy chain).

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

“Fv” is the minimum antibody fragment, which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains, which enables the scFv to form the desired structure for antigen binding. For a review of scFv, see Plückthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing scFv fragments (see preceding paragraph) typically with short linkers (such as about 5 to about 10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” scFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Plückthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues, which encompass the CDRs as defined by each of the above-cited references, are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present invention and for possible inclusion in one or more claims herein. Unless otherwise defined, the CDR sequences provided herein are based on Chothia definition.

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The expression “variable-domain residue-numbering as in Chothia” or “amino-acid-position numbering as in Chothia,” and variations thereof, refers to the numbering system used for heavy-chain variable domains or light-chain variable domains of the compilation of antibodies in Chothia et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Chothia) after residue 52 of H2 and inserted residues (e.g. residues 82a, 82b, and 82c, etc. according to Chothia) after heavy-chain FR residue 82. The Chothia numbering of residues may be determined for a given antibody by alignment at regions of homology of the sequence of the antibody with a “standard” Chothia t numbered sequence.

“Framework” or “FR” residues are those variable-domain residues other than the CDR residues as herein defined.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual, (Cold Spring Harbor Laboratory Press, 2nd ed. 1988); Hammerling et al., Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995)).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino acid sequence, which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol. 227:381 (1991); Marks et al., J. Mol. Biol. 222:581 (1991). Also available for the preparation of human monoclonal antibodies are methods described in Cole et al., Monoclonal Antibodies and Cancer Therapy, Alan R. Liss, 77 (1985); Boerner et al., J. Immunol. 147(1):86-95 (1991). See also van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA 103:3557-3562 (2006) regarding human antibodies generated via a human B-cell hybridoma technology.

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of <1 M, <100 nM, <10 nM, 1 nM, or <0.1 nM. In certain embodiments, an antibody specifically binds to an epitope on a protein that is conserved among the protein from different species. In another embodiment, specific binding can include, but does not require exclusive binding.

The term “specificity” refers to selective recognition of an antigen binding protein (such as a chimeric receptor or an antibody construct) for a particular epitope of an antigen. Natural antibodies, for example, are monospecific. The term “multispecific” as used herein denotes that an antigen binding protein has two or more antigen-binding sites of which at least two bind different antigens or epitopes. “Bispecific” as used herein denotes that an antigen binding protein has two different antigen-binding specificities. The term “monospecific” as used herein denotes an antigen binding protein that has one or more binding sites each of which bind the same antigen or epitope.

The term “valent” as used herein denotes the presence of a specified number of binding sites in an antigen binding protein. A natural antibody for example or a full-length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

“Chimeric antigen receptor” or “CAR” as used herein refers to genetically engineered receptors, which graft one or more antigen specificity onto cells, such as T cells. CARs are also known as “artificial T-cell receptors,” “chimeric T cell receptors,” or “chimeric immune receptors.” In some embodiments, the CAR comprises an extracellular variable domain of an antibody specific for a tumor antigen, and an intracellular signaling domain of a T cell receptor and/or other receptors, such as one or more costimulatory domains. “CAR-T” refers to a T cell that expresses a CAR.

“T cell receptor” or “TCR” as used herein refers to endogenous or recombinant T cell receptor comprising an extracellular antigen binding domain that binds to a specific antigenic peptide bound in an MHC molecule. In some embodiments, the TCR comprises a TCRα polypeptide chain and a TCR β polypeptide chain. In some embodiments, the TCR specifically binds a tumor antigen. “TCR-T” refers to a T cell that expresses a recombinant TCR.

“Chimeric T cell receptor” or “cTCR” as used herein refers to an engineered receptor comprising an extracellular antigen-binding domain that binds to a specific antigen, a transmembrane domain of a first subunit of the TCR complex or a portion thereof, and an intracellular signaling domain of a second subunit of the TCR complex or a portion thereof, wherein the first or second subunit of the TCR complex is a TCRα chain, TCRβ chain, TCRγ chain, TCRδ chain, CD3ε, CD3δ, or CD3γ. The transmembrane domain and the intracellular signaling domain of a cTCR may be derived from the same subunit of the TCR complex, or from different subunits of the TCR complex. The intracellular domain may be the full-length intracellular signaling domain or a portion of the intracellular domain of a naturally occurring TCR subunit. In some embodiments, the cTCR comprises the extracellular domain of the TCR subunit or a portion thereof. In some embodiments, the cTCR does not comprise the extracellular domain of the TCR subunit. An “eTCR” refers to a cTCR comprising an extracellular domain of CD3ε.

“Percent (%) amino acid sequence identity” with respect to a polypeptide sequence are defined as the percentage of amino acid residues in a candidate sequence that are identical with the amino acid residues in the specific polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full-length of the sequences being compared. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

The term “express” refers to translation of a nucleic acid into a protein. Proteins may be expressed and remain intracellular, become a component of the cell surface membrane, or be secreted into extracellular matrix or medium.

The term “host cell” refers to a cell that can support the replication or expression of the expression vector. Host cells may be prokaryotic cells such as E. coli, or eukaryotic cells, such as yeast, insect cells, amphibian cells, or mammalian cells.

The term “transfected” or “transformed” or “transduced” as used herein refers to a process by which exogenous nucleic acid is transferred or introduced into the host cell. A “transfected” or “transformed” or “transduced” cell is one that has been transfected, transformed or transduced with exogenous nucleic acid.

The term “in vivo” refers to inside the body of the organism from which the cell is obtained. “Ex vivo” or “in vitro” means outside the body of the organism from which the cell is obtained.

The term “cell” includes the primary subject cell and its progeny.

“Activation”, as used herein in relation to a cell expressing CD3, refers to the state of the cell that has been sufficiently stimulated to induce a detectable increase in downstream effector functions of the CD3 signaling pathway, including, without limitation, cellular proliferation and cytokine production.

As used herein, the term “autologous” is meant to refer to any material derived from the same individual to whom it is later to be re-introduced into the individual.

“Allogeneic” refers to a graft derived from a different individual of the same species.

As used herein, “deplete” includes a reduction by at least 75%, at least 80%, at least 90%, at least 99%, or 100%.

The term “domain” when referring to a portion of a protein is meant to include structurally and/or functionally related portions of one or more polypeptides that make up the protein. For example, a transmembrane domain of an immune cell receptor may refer to the portions of each polypeptide chain of the receptor that span the membrane. A domain may also refer to related portions of a single polypeptide chain. For example, a transmembrane domain of a monomeric receptor may refer to portions of the single polypeptide chain of the receptor that span the membrane. A domain may also include only a single portion of a polypeptide.

The term “isolated nucleic acid” as used herein is intended to mean a nucleic acid of genomic, cDNA, or synthetic origin or some combination thereof, which by virtue of its origin the “isolated nucleic acid” (1) is not associated with all or a portion of a polynucleotide in which the “isolated nucleic acid” is found in nature, (2) is operably linked to a polynucleotide which it is not linked to in nature, or (3) does not occur in nature as part of a larger sequence.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “inducible promoter” refers to a promoter whose activity can be regulated by adding or removing one or more specific signals. For example, an inducible promoter may activate transcription of an operably linked nucleic acid under a specific set of conditions, e.g., in the presence of an inducing agent or conditions that activates the promoter and/or relieves repression of the promoter.

As used herein, “treatment” or “treating” is an approach for obtaining beneficial or desired results, including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of the disease (such as, for example, tumor volume in cancer). The methods of the invention contemplate any one or more of these aspects of treatment.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

Administration “in combination with” one or more further agents includes simultaneous and sequential administration in any order.

The term “simultaneous” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered with a time separation of no more than about 15 minutes, such as no more than about any of 10, 5, or 1 minute.

The term “sequentially” is used herein to refer to administration of two or more therapeutic agents where the administration of one or more therapeutic agent(s) continues after discontinuing the administration of one or more other agent(s). For example, administration of the two or more agents are administered with a time separation of more than about 15 minutes, such as about any of 20, 30, 40, 50, or 60 minutes, 1 day, 2 days, 3 days, 1 week, 2 weeks, or 1 month, or longer.

A “subject” or an “individual” for purposes of treatment refers to any animal classified as a mammal, including humans, domestic and farm animals, and zoo, sports, or pet animals, such as dogs, horses, cats, cows, etc.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

As used herein and in the appended claims, the singular forms “a,” “an,” and “the” include plural referents unless the context clearly dictates otherwise.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

### Engineered Immune Cells Comprising Recognition Molecules

The present application provides engineered immune cells comprising on its surface a recognition molecule that comprises a binding moiety specifically binding to a target molecule on the surface of a target cell, wherein the target molecule comprises an extracellular domain, and wherein the immune cell is capable of killing a target cell that comprises on its surface the target molecule. In one aspect, the binding moiety specifically binds to a distal portion of the extracellular domain, and the immune cell is capable of killing a target cell that comprises on its surface both the target molecule and the recognition molecule. In another aspect, the binding moiety specifically binds to a proximal portion of the extracellular domain, and the engineered immune cell has no or reduced capability of killing a target cell comprising on its surface both the target molecule and the recognition molecule.

Recognition molecules described herein can be any binding-moiety containing molecules present on the surface of an engineered immune cell. In some embodiments, the recognition molecule is an immune cell receptor molecule comprising an extracellular domain comprising the binding moiety, a transmenbrane domain, and a signaling domain. Suitable immune cell receptors include, for example, chimeric antigen receptor and chimeric T cell receptor.

In some embodiments, the binding moiety is an antibody or fragment thereof. In some embodiments, the binding moiety is a peptide ligand.

In some embodiments, the distance from the distal portion of the extracellular domain to the membrane of the target cell is more than about 0.5 times (e.g., more than about 1 time, more than about 1.5 times, 2 times, or more) of the distance from the binding moiety to the membrane of engineered immune cell. In some embodiments, the distance from the proximal portion of the extracellular domain to the membrane of the target cell is no more than about 2 times (e.g., no more than about 1.5 times, or no more than about 1 time) of the distance from the binding moiety to the membrane of engineered immune cell.

In some embodiments, the distal portion of the extracellular domain is at least about 30 Å (e.g., at least about 40, 60, 90, 120 or more Å) away from the membrane of the target cell. In some embodiments, the proximal portion of the extracellular domain is no more than about 120 Å (e.g., no more than about 100, 90, 80, 70 or 60 Å) from the membrane of the target cell.

In some embodiments, the extracellular domain of the target molecule is at least about 100 amino acids long, including for example at least about 110, 120, 130, 140, 150, 160, 170, 175, 180, 190, or 200 amino acids long. In some embodiments, the extracellular domain of the target molecule comprises two or more, three or more, four or more, five or more, six or more, seven or more, or eight or more IgG-like domains. In some embodiments, the target molecule is a transmembrane receptor.

Suitable target molecules described herein include, but are not limited to, CD22, CD4, CD21 (CR2), CD30, ROR1, CD5, and CD20. The target molecule may have two or more repeats (e.g., Ig-like domains) in its extracellular domain.

CD4, also known as Cluster of Differentiation 4, is a glycoprotein found on the surface of immune cells, particularly CD4+, or helper, T cells. CD4 is an important cell-surface molecule required for HIV-1 entry and infection. HIV-1 entry is triggered by interaction of the viral envelope (Env) glycoprotein gp120 with domain 1 (D1) of the T-cell receptor CD4. As HIV infection progresses, greater numbers of CD4+ T cells are targeted and destroyed by the virus, resulting in an increasingly compromised immune system; CD4+ T cell count is therefore used as a proxy for the progression and stage of HIV/AIDS in an individual. Furthermore, HIV gene products Env, Vpu, and Nef, are involved in the downregulation of CD4 during HIV infection (see Tanaka, M., et al. Virology (2003) 311(2):316-325).

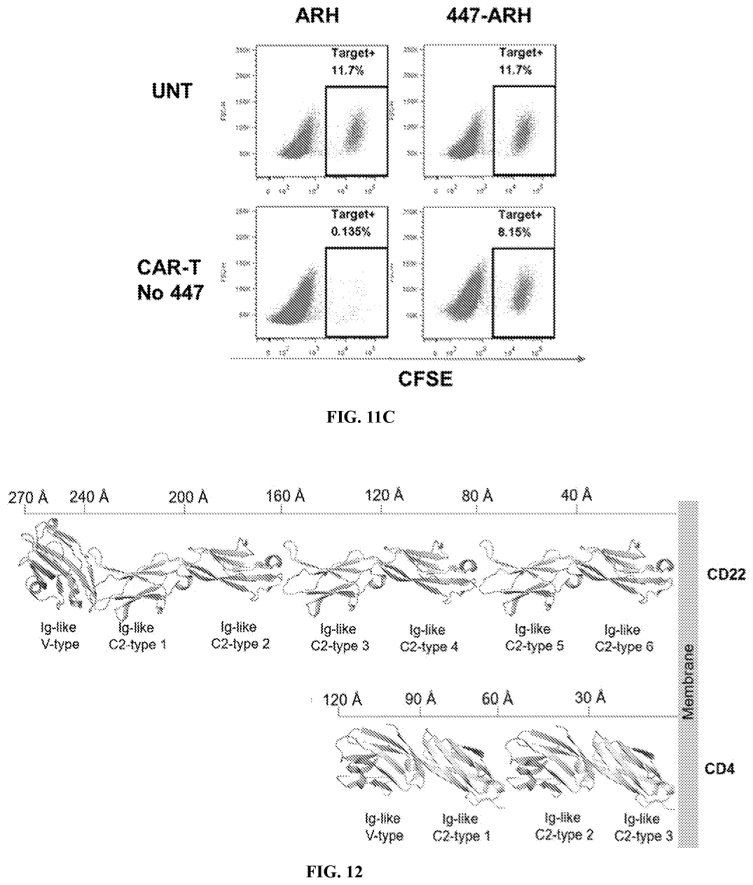

CD4 is a member of the immunoglobulin superfamily, and has four extracellular immunoglobulin domains. As shown in FIG. 12, the extracellular domain of CD4 includes, from the N-terminus to the C-terminus, Ig-like V-type domain (“Domain 1” or D1; amino acid residues 26-125), Ig-like C2-type 1 domain (“Domain 2” or D2; amino acid residues 126-203), Ig-like C2-type 2 domain (“Domain 3” or D3; amino acid residues 204-317), and Ig-like C2-type 3 domain (“Domain 4” or D4; amino acid residues 318-374), wherein the amino acid residue positions are based on the full-length amino acid sequence of human CD4 (UniProtKB ID: P01730), e.g., SEQ ID NO: 45. D1 and D3 show similarity to immunoglobulin variable domains, while D2 and D4 show similarity to immunoglobulin constant domains.

CD22, also known as B-cell receptor CD22, is a cell surface receptor that binds sialytated glycoproteins (e.g., CD45) and mediates B-cell/B-cell interactions. As shown in FIG. 12, the extracellular domain of CD22 has 7 Ig-like domains, including, from the N-terminus to the C-terminus, Ig-like V-type domain (“Domain 1” or “D1”; amino acid residues 20-138), Ig-like C2-type 1 domain (“Domain 2” or “D2”; amino acid residues 143-235), Ig-like C2-type 2 domain (“Domain 3” or “D3”; amino acid residues 242-326), Ig-like C2-type 3 domain (“Domain 4” or “D4”; amino acid residues 331-416), Ig-like C2-type 4 domain (“Domain 5” or “D5”; amino acid residues 419-500), Ig-like C2-type 5 domain (“Domain 6” or “D6”; amino acid residues 505-582), and Ig-like C2-type 6 domain (“Domain 7” or “D7”; amino acid residues 593-676), wherein the amino acid residue positions are based on the full-length amino acid sequence of human CD22 (UniProtKB ID: P20273), e.g., SEQ ID NO: 66.

CD21, also known as complement receptor type 2 (CR2), is a receptor for complement C3, for the Epstein-Barr virus on human B-cells and T-cells and for HNRNPU. CD21 participates in B lymphocytes activation. The extracellular domain of CD21 has 15 Sushi domains, including, from the N-terminus to the C-terminus, Sushi 1 (amino acid residues 21-84), Sushi 2 (amino acid residues 89-148), Sushi 3 (amino acid residues 152-212), Sushi 4 (amino acid residues 213-273), Sushi 5 (amino acid residues 274-344), Sushi 6 (amino acid residues 349-408), Sushi 7 (amino acid residues 409-468), Sushi 8 (amino acid residues 469-524), Sushi 9 (amino acid residues 525-595), Sushi 10 (amino acid residues 600-659), Sushi 11 (amino acid residues 660-716), Sushi 12 (amino acid residues 717-781), Sushi 13 (amino acid residues 786-845), Sushi 14 (amino acid residues 849-909), and Sushi 15 (amino acid residues 910-970), wherein the amino acid residue positions are based on the full-length amino acid sequence of human CD21 (UniProtKB ID: P20023).

CD30, also known as tumor necrosis factor receptor superfamily member 8 (TNFRSF8), is a receptor for TNFSF8/CD30L. CD30 may play a role in the regulation of cellular growth and transformation of activated lymphoblasts. It regulates gene expression through activation of NF-kappa-B. The extracellular domain of CD30 has 6 TNFR-Cys domains, including, from the N-terminus to the C-terminus, TNFR-Cys domain 1 (amino acid residues 28-66), TNFR-Cys domain 2 (amino acid residues 68-106), TNFR-Cys domain 3 (amino acid residues 107-150), TNFR-Cys domain 4 (amino acid residues 205-241), TNFR-Cys domain 5 (amino acid residues 243-281), and TNFR-Cys domain 6 (amino acid residues 282-325), wherein the amino acid residue positions are based on the full-length amino acid sequence of human CD30 (UniProtKB ID: P28908).

ROR1, also known as inactive tyrosine-protein kinase transmembrane receptor ROR1, is a receptor for ligand WNT5 Å that activate downstream NFkB signaling pathway and may result in the inhibition of WNT3A-mediated signaling. The extracellular domain of ROR1 includes various subdomains, including, from the N-terminus to the C-terminus, Ig-like C2-type domain (amino acid residues 42-147), FZ domain (amino acid residues 165-299), and Kringle domain (amino acid residues 312-391), wherein the amino acid residue positions are based on the full-length amino acid sequence of human ROR1 (UniProtKB ID: Q01973).

CD5, also known as T-cell surface glycoprotein CD5, may act as a receptor in regulating T-cell proliferation. The extracellular domain of CD5 has 3 SRCR domains, including, from the N-terminus to the C-terminus, SRCR 1 (amino acid residues 35-133), SRCR 2 (amino acid residues 159-268), and SRCR 3 (amino acid residues 276-368), wherein the amino acid residue positions are based on the full-length amino acid sequence of human CD5 (UniProtKB ID: P06127).

CD20, also known as B-lymphocyte antigen CD20 or MS4A1, is a B-lymphocyte-specific membrane protein that plays a role in the regulation of cellular calcium influx necessary for the development, differentiation, and activation of B-lymphocytes. CD20 has two extracellular domains at amino acid residues 79-84 and 142-188, wherein the amino acid residue positions are based on the full-length amino acid sequence of human CD20 (UniProtKB ID: P11836).

Target cells can be any cells whose expresses a target molecule (such as the exemplary target molecules described herein). In some embodiments, the target cell is an immune cell. In some embodiments, the target cell is a tumor cell.

### Recognition Molecules Comprising a Binding Moiety Specifically Binding to a Distal Portion of the Extracellular Domain of a Target Molecule (“Distal Portion Recognition Molecules”)

The present application in some embodiments provides an engineered immune cell comprising on its surface a recognition molecule that comprises a binding moiety specifically binding to a target molecule on the surface of a target cell, wherein the target molecule comprises an extracellular domain, wherein the binding moiety specifically binds to a distal portion of the extracellular domain, wherein the immune cell is capable of killing a target cell that comprises on its surface the target molecule, and wherein the immune cell is capable of killing a target cell that comprises on its surface both the target molecule and the recognition molecule. In some embodiments, the engineered immune cell is capable of killing a target cell that comprises on its surface both the target molecule and the recognition molecule by at least 2 fold, such as at least about 3 fold, at least about 4 fold, at least about 5 fold, at least about 10 fold, or more as compared to an engineered immune cell comprising on its surface a recognition molecule comprising a binding moiety that binds to a proximal portion of the extracellular domain of the target molecule.

The binding moiety can be, but is not limited to, an sdAb (e.g., VHH), an scFv, a Fab′, a (Fab′)₂, an Fv, or a peptide ligand.

We have demonstrated that engineered immune cells containing an anti-CD4 D1 immune cell receptor (i.e., immune cell receptor having a binding moiety specifically recognizing Domain 1 of CD4) are able to kill themselves. We have further demonstrated that engineered immune cells containing an anti-CD22 D1-4 immune cell receptors (i.e., immune cell receptor having a binding moiety specifically recognizing Domains 1-4 of CD22) are also able to kill themselves. Without being bound by theory, it is believed that the anti-CD4 D1 moiety and anti-CD22 D1-4 moiety in an engineered immune cell may be too far away from intrinsic CD4 or CD22 on the same cell to block the recognition of Domain 1 of CD4 or Domains 1-4 of CD22 by another engineered immune cell, respectively, thus leading to killing of the engineered immune cell. Similarly, other recognition molecules having a binding moiety that binds to a distal portion of a target molecule would possess the same property as the anti-CD4 D1 and anti-CD22 D1-4 molecules described herein. These recognition molecules are thus particularly useful for autologous therapy, where it is desirable to remove autologous cells expressing the immune cell receptors.

In some embodiments, the binding moiety binds to a region (e.g. an epitope) in the extracellular domain that is about 50 amino acids or more away from the C-terminus of the extracellular domain. “C-terminus of the extracellular domain regards” refers to the C-terminal end of the extracellular domain immediately, and is also referred to as the “juxtamembrane residue” on the target molecule. When the target molecule is a transmembrane receptor, the juxtamembrane residue is immediately followed by the first residue in the transmembrane domain. The distal portion-recognition molecule binds to a region that is far enough away from the juxtamembrane residue of the target molecule such that it would not be able to block other binding moieties from binding to the target molecule when co-expressed with the target molecule. In some embodiments, the binding moiety binds to a region (e.g., epitope) in the extracellular domain that is about 55, 60, 70, 80, 90, 100, 110, 120, 130, 140, 150 amino acids or more away from the C-terminus of the extracellular domain.

In some embodiments, the binding moiety binds to a region (e.g., an epitope) within about 130 (such as within about any of 120, 110, 100, 90, 80, 70, 60, 50, 40, or 30) amino acids from the N-terminus of the extracellular domain of the target molecule. In some embodiments, the binding moiety binds to an epitope that falls within any one or more of the following regions: amino acid residues 26-125, 26-46, 46-66, 66-86, 86-106, and 106-125 from the N-terminus of the extracellular domain of the target molecule. In some embodiments, the binding moiety binds to a region (e.g. an epitope) within about 80 amino acids from the N-terminus of the extracellular domain.

In some embodiments, the target molecule comprises three or more Ig-like domains, and the binding moiety binds to a region (e.g. an epitope) outside the first Ig-like domains from the C-terminal end of the extracellular domain. In some embodiments, the binding moiety binds to a region (e.g., an epitope) that is within the first Ig-like domain at the N-terminal end of the extracellular domain.

In some embodiments, the binding moiety of the recognition molecule binds to the target molecule between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any values and ranges between these values).

### Anti-CD22 D1-4 Binding Moieties

In some embodiments, the CD22 binding moiety of the anti-CD22 D1-4 recognition molecule binds to Domains 1-4 (D1-4) of CD22 with a Kd between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any values and ranges between these values). In some embodiments, the CD22 is human CD22. In some embodiments, the CD22 comprises the amino acid sequence of SEQ ID NO: 66.

In some embodiments, the anti-CD22 D1-4 binding moiety binds to an epitope in D1 of CD22. In some embodiments, the anti-CD22 D1-4 binding moiety binds to an epitope in D2 of CD22. In some embodiments, the anti-CD22 D1-4 binding moiety binds to an epitope in D3 of CD22. In some embodiments, the anti-CD22 D1-4 binding moiety binds to an epitope in D4 of CD22. In some embodiments, the anti-CD22 D1-4 binding moiety binds to an epitope that bridges any two or more domains among D1-D4 of CD22. In some embodiments, the anti-CD22 D1-4 binding moiety binds to an epitope within amino acid residues 20-416 of SEQ ID NO: 66. In some embodiments, the anti-CD22 D1-4 binding moiety binds to an epitope that falls within any one or more of the following regions: amino acid residues 20-138, 143-235, 242-326, and 331-416 of SEQ ID NO: 66. In some embodiments, the CD22 is human CD22.

In some embodiment, the CD22 binding moiety binds to an epitope that is at least about 50 amino acid residues, such as at least about any one of 60, 70, 80, 90, 100, 120, 140, 160, 180, 200, 220, 240, or 260 amino acid residues, including any values and ranges in between these values, away from the C-terminus of the extracellular domain of CD22. In some embodiments, the CD22 binding moiety binds to an epitope that is within about any one of 400, 380, 360, 340, 320, 300, 280, 260, 240, 220, 200, 180, 160, 140, 120, 100, 90, 80, 70, 60, 50 or fewer amino acid residues, including any values and ranges in between these values, away from the N-terminus of the extracellular domain of CD22.

In some embodiment, the CD22 binding moiety binds to an epitope of a CD22 molecule that is at least about 30, 40, 50, 60, 70, 80, 90, 100, 120, 130, 140, 150, 160, 170, 180, 190, 200, 210, 220, 230, 240, 250, 260 or more A away from the membrane of a target cell that expresses CD22. In some embodiments, the CD22 binding moiety binds to an epitope of a CD22 molecule that is about 30-40, 40-80, 80-120, 120-160, 160-200, 200-240, 240-270, 30-80, 30-120, 60-120, 60-160, 60-100, 90-120, 100-200, 100-150, or 30-270 Å away from the membrane of a target cell that expresses CD22.

In some embodiments, the distance from the CD22 epitope in D1-D4 is more than about 0.5 times, 1 time, 1.5 times, 2 times, 2.5 times, 3 times or more, including any values and ranges between these values, of the distance from the CD22 binding moiety to the membrane of the engineered immune cell.

In some embodiments, the CD22 binding moiety is derived from RFB4 or a humanized variant thereof, for example as described in U.S. Pat. No. 9,139,649. In some embodiments, the CD22 binding moiety competes for binding against RFB4. In some embodiments, the CD22 binding moiety binds to the same or overlapping epitope as that of RFB4. In some embodiments, the CD22 binding moiety comprises one, two, three, four, five, or six heavy chain and light chain complementary determining regions (CDRs) of RFB4 or a humanized variant thereof. In some embodiments, the CD22 binding moiety comprises the heavy chain variable domain (VH) and/or the light chain variable domain (VL) of RFB4 or a humanized variant thereof.

In some embodiments, the CD22 binding moiety is derived from Epratuzumab or a biosimilar thereof, for example as described in U.S. Pat. No. 7,074,403 or 9,139,649. In some embodiments, the CD22 binding moiety competes for binding against Epratuzumab. In some embodiments, the CD22 binding moiety binds to the same or overlapping epitope as that of Epratuzumab. In some embodiments, the CD22 binding moiety comprises one, two, three, four, five, or six heavy chain and light chain complementary determining regions (CDRs) of Epratuzumab. In some embodiments, the CD22 binding moiety comprises the heavy chain variable domain (VH) and/or the light chain variable domain (VL) of Epratuzumab.

In some embodiments, the CD22 binding moiety of the anti-CD22 D1-4 recognition molecule competes for binding with a reference antibody that specifically binds to an epitope within Domains 1-4 (D1-4) of CD22 (“anti-CD22 D1-4 antibody”), or binds to an epitope in D1-4 of CD22 that overlaps with the binding epitope of a reference anti-CD22 D1-4 antibody. In some embodiments, the CD22 binding moiety comprises the same heavy chain and light chain CDR sequences as those of a reference anti-CD22 D1-4 antibody. In some embodiments, the CD22 binding moiety comprises a VH sequence that has at least about 80% (such as at least about 85%, 90%, 95%, 96%, 97%, 98%, or 99%) sequence identity as the VH sequence of a reference anti-CD22 D1-4 antibody, and/or a VL sequence that has at least about 80% (such as at least about 85%, 90%, 95%, 96%, 97%, 98%, or 99%) sequence identity as the light chain variable sequence of a reference anti-CD22 D1-4 antibody. In some embodiments, the CD22 binding moiety comprises the same heavy chain and light chain variable sequences as those of a reference anti-CD22 D1-4 antibody.

Any antibodies that are known to specifically recognize Domains 1-4 of CD22 can serve as a reference antibody, including, but not limited to, hLL2 (Epratuzumab), Inotuzumab (Pfizer, Groton, Conn.), BL22 (Cambridge Antibody Technology Group, Cambridge, England), HA22 (Cambridge Antibody Technology Group, Cambridge, England), HB22.7 (Duke University, Durham, N.C.) and RFB4 (e.g., Invitrogen, Grand Island, N.Y.; Santa Cruz Biotechnology, Santa Cruz, Calif.)

In some embodiments, the reference antibody is RFB4 or a humanized variant thereof. In some embodiments, the reference anti-CD22 D1-4 antibody comprises a heavy chain CDR1 (HC-CDR1) comprising the amino acid sequence of SEQ ID NO: 67, a heavy chain CDR2 (HC-CDR2) comprising the amino acid sequence of SEQ ID NO: 68, a heavy chain CDR3 (HC-CDR3) comprising the amino acid sequence of SEQ ID NO: 69, a light chain CDR1 (LC-CDR1) comprising the amino acid sequence of SEQ ID NO: 70, a light chain CDR2 (LC-CDR2) comprising the amino acid sequence of SEQ ID NO: 71, and a light chain CDR3 (LC-CDR3) comprising the amino acid sequence of SEQ ID NO: 72. In some embodiments, the reference anti-CD22 D1-4 antibody comprises a heavy chain variable domain (VH) comprising the amino acid sequence of SEQ ID NO: 73 and a light chain variable domain (VL) comprising the amino acid sequence of SEQ ID NO: 74.

In some embodiments, the CD22 binding moiety comprises a VH comprising a HC-CDR1 comprising SEQ ID NO: 67, a HC-CDR2 comprising SEQ ID NO: 68, a HC-CDR3 comprising SEQ ID NO: 69; and a VL comprising a LC-CDR1 comprising SEQ ID NO: 70, a LC-CDR2 comprising SEQ ID NO: 71, and a LC-CDR3 comprising SEQ ID NO: 72. In some embodiments, the CD22 binding moiety comprises a VH comprising HC-CDR1, HC-CDR2 and HC-CDR3 of SEQ ID NO: 73, and a VL comprising LC-CDR1, LC-CDR3 and LC-CDR3 of SEQ ID NO: 74. In some embodiments, the CD22 binding moiety comprises a VH comprising an amino acid sequence having at least about 80% (e.g., at least about any one of 85%, 90%, 95%, 98%, 99%, or more) sequence identity to SEQ ID NO: 73, and a VL comprising an amino acid sequence having at least about 80% (e.g., at least about any one of 85%, 90%, 95%, 98%, 99%, or more) sequence identity to SEQ ID NO: 74. In some embodiments, the CD22 binding moiety comprises a VH comprising SEQ ID NO: 73 and a VL comprising SEQ ID NO: 74.

### Recognition Molecules Comprising a Binding Moiety Specifically Binding to a Proximal Portion of the Extracellular Domain of a Target Molecule (“Proximal Portion Recognition Molecules”)

The present application in some embodiments provides an engineered immune cell comprising on its surface a recognition molecule that comprises a binding moiety specifically binding to a target molecule on the surface of a target cell, wherein the target molecule comprises an extracellular domain, wherein the binding moiety specifically binds to a proximal portion of the extracellular domain, wherein the engineered immune cell is capable of killing a target cell that comprises on its surface the target molecule, and wherein the engineered immune cell has no or reduced capability of killing a target cell comprising on its surface both the target molecule and the recognition molecule. In some embodiments, the engineered immune cell kills a target cell that comprises on its surface both the target molecule and the recognition molecule by no more than about 20% as compared to an engineered immune cell comprising on its surface a recognition molecule comprising a binding moiety that binds to a distal end of the extracellular domain of the target molecule.

The binding moiety can be, but is not limited to, an sdAb (e.g., VHH), an scFv, a Fab′, a (Fab′)₂, an Fv, or a peptide ligand.

We have demonstrated that engineered immune cells containing an anti-CD4 D2/D3 immune cell receptor (i.e., immune cell receptor having a binding moiety specifically recognizing Domains 2/3 of CD4) are unable to kill themselves. We have further demonstrated that engineered immune cells containing an anti-CD22 D5-7 immune cell receptors (i.e., immune cell receptor having a binding moiety specifically recognizing Domains 5-7 of CD22) are also unable to kill themselves. Without being bound by theory, it is believed that the anti-CD4 D2/D3 moiety and the anti-CD22 D5-7 moiety in an engineered immune cell may be within a proper distance from intrinsic CD4 or CD22 on the same cell to block recognition of D2/3 of CD4 or D5-7 of CD22 by another engineered immune cell respectively, thus protecting the engineered immune cell from being attacked. Similarly, other recognition molecules having a binding moiety that binds to a proximal portion of a target molecule would possess the same property as the anti-CD4 D2/D3 and anti-CD22 D5-7 molecules described herein. These recognition molecules are thus particularly useful for allogeneic therapy, where it is desirable for cells comprising the recognition molecules to persist throughout the treatment.

In some embodiments, the binding moiety binds outside a region that is about 80 (such as about any of 85, 90, 100, 110, 120, or more) amino acids away from the N-terminus of the extracellular domain of the target molecule.

In some embodiments, the binding moiety binds to a region (e.g. an epitope) in the extracellular domain that is within about 120 (such as 115, 110, 105, or 102) amino acids from the C-terminus of the extracellular domain. In some embodiments, the binding moiety binds to a region (e.g. an epitope) in the extracellular domain that is within about 120 (such as 102, 100, 90, 80, 70, 60, or 50) amino acids from the C-terminus of the extracellular domain. In some embodiments, the binding moiety binds to a region (e.g. an epitope) in the extracellular domain that is within about 50 amino acids from the C-terminus of the extracellular domain. In some embodiments, the binding moiety binds to an epitope that falls within any one or more of the following regions (e.g., epitopes): amino acid residues 26-125, 26-46, 46-66, 66-86, 86-106, and 106-125 from the C-terminus of the extracellular domain of the target molecule.

In some embodiments, the extracellular domain of the target molecule comprises two or more Ig-like domains, and the binding moiety binds to a region outside the first Ig-like domain at the N-terminal end of the extracellular domain. In some embodiments, the extracellular domain of the target molecule comprises two or more Ig-like domains, and the binding moiety binds to a region (e.g. epitope) within the first Ig-like domain at the N-terminal end of the extracellular domain.

In some embodiments, the binding moiety of the recognition molecule binds to the target molecule between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any values and ranges between these values).

### Anti-CD22 D5-7 Binding Moieties

In some embodiments, the CD22 binding moiety of the anti-CD22 D5-7 recognition molecule binds to D5-7 of CD22 with a Kd between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any values and ranges between these values). In some embodiments, the CD22 is human CD22. In some embodiments, the CD22 comprises the amino acid sequence of SEQ ID NO: 66.

In some embodiments, the anti-CD22 D5-7 binding moiety binds to an epitope in D5 of CD22. In some embodiments, the anti-CD22 D5-7 binding moiety binds to an epitope in D6 of CD22. In some embodiments, the anti-CD22 D5-7 binding moiety binds to an epitope in D7 of CD22. In some embodiments, the anti-CD22 D5-7 binding moiety binds to an epitope that bridges any two or more domains among D5-D7 of CD22. In some embodiments, the anti-CD22 D5-7 binding moiety binds to an epitope within amino acid residues 419-676 of SEQ ID NO: 66. In some embodiments, the CD22 binding moiety of the anti-CD22 D5-7 immune cell receptor binds to an epitope that falls within any one or more of the following regions: amino acid residues 419-500, 505-582 and 593-676 of SEQ ID NO: 66.

In some embodiment, the CD22 binding moiety binds to an epitope that is at least about 80 amino acid residues, such as at least about any one of 90, 100, 120, 140, 160, 180, 200, 220, 240, 260, 280, 300, 320, 340, 360, 380, 400 or more amino acid residues, including any values and ranges in between these values, away from the N-terminus of the extracellular domain of CD22. In some embodiments, the CD22 binding moiety binds to an epitope that is within about any one of 260, 240, 220, 200, 180, 160, 140, 120, 100, 90, 80, 70, 60, 50 or fewer amino acid residues, including any values and ranges in between these values, away from the C-terminus of the extracellular domain of CD22.

In some embodiment, the CD22 binding moiety binds to an epitope of a CD22 molecule that is no more than about 140, 130, 120, 110, 100, 90, 80, 70, 60, 50, 40, 30 or fewer A away from the membrane of a target cell that expresses CD22. In some embodiments, the CD22 binding moiety binds to an epitope of a CD22 molecule that is about 0-30, 0-40, 0-80, 0-120, 30-60, 60-80, 80-120, 40-80, or 30-120 Å away from the membrane of a target cell that expresses CD22.

In some embodiments, the distance from the CD22 epitope in D5-D7 is no more than about 2 times, 1.75 times, 1.5 times, 1.25 times, 1 time or less, including any values and ranges between these values, of the distance from the CD22 binding moiety to the membrane of the engineered immune cell.

In some embodiments, the CD22 binding moiety is derived from m971 or m972, for example as described in U.S. Ser. No. 10/494,435. In some embodiments, the CD22 binding moiety competes for binding against m971. In some embodiments, the CD22 binding moiety binds to the same or overlapping epitope as that of m971. In some embodiments, the CD22 binding moiety comprises one, two, three, four, five, or six heavy chain and light chain complementary determining regions (CDRs) of m971. In some embodiments, the CD22 binding moiety comprises the VH and/or the VL of m971.

In some embodiments, the CD22 binding moiety of the anti-CD22 D5-7 recognition molecule competes for binding with a reference antibody that specifically binds to an epitope within D5-7 of CD22 (“anti-CD22 D5-7 antibody”), or binds to an epitope in D5-7 of CD22 that overlaps with the binding epitope of a reference anti-CD22 D5-7 antibody. In some embodiments, the CD22 binding moiety comprises the same heavy chain and light chain CDR sequences as those of a reference anti-CD22 D5-7 antibody. In some embodiments, the CD22 binding moiety comprises a VH sequence that has at least about 80% (such as at least about 85%, 90%, 95%, 96%, 97%, 98%, or 99%) sequence identity as the VH sequence of a reference anti-CD22 D5-7 antibody, and/or a VL sequence that has at least about 80% (such as at least about 85%, 90%, 95%, 96%, 97%, 98%, or 99%) sequence identity as the light chain variable sequence of a reference anti-CD22 D5-7 antibody. In some embodiments, the CD22 binding moiety comprises the same heavy chain and light chain variable sequences as those of a reference anti-CD22 D5-7 antibody.

Any antibodies that are known to specifically recognize Domains D5-7 of CD22 can serve as a reference antibody, including, but not limited to, m971 and m972. In some embodiments, the reference antibody is m971. In some embodiments, the reference anti-CD22 D5-7 antibody comprises a HC-CDR1 comprising the amino acid sequence of SEQ ID NO: 76, a HC-CDR2 comprising the amino acid sequence of SEQ ID NO: 77, a HC-CDR3 comprising the amino acid sequence of SEQ ID NO: 78, a LC-CDR1 comprising the amino acid sequence of SEQ ID NO: 79, a LC-CDR2 comprising the amino acid sequence of SEQ ID NO: 80, and a LC-CDR3 comprising the amino acid sequence of SEQ ID NO: 81. In some embodiments, the reference anti-CD22 D5-7 antibody comprises a VH comprising the amino acid sequence of SEQ ID NO: 82 and a VL comprising the amino acid sequence of SEQ ID NO: 83.

In some embodiments, the CD22 binding moiety comprises a VH comprising a HC-CDR1 comprising SEQ ID NO: 76, a HC-CDR2 comprising SEQ ID NO: 77, a HC-CDR3 comprising SEQ ID NO: 78; and a VL comprising a LC-CDR1 comprising SEQ ID NO: 79, a LC-CDR2 comprising SEQ ID NO: 80, and a LC-CDR3 comprising SEQ ID NO: 81. In some embodiments, the CD22 binding moiety comprises a VH comprising HC-CDR1, HC-CDR2 and HC-CDR3 of SEQ ID NO: 82, and a VL comprising LC-CDR1, LC-CDR3 and LC-CDR3 of SEQ ID NO: 83. In some embodiments, the CD22 binding moiety comprises a VH comprising an amino acid sequence having at least about 80% (e.g., at least about any one of 85%, 90%, 95%, 98%, 99%, or more) sequence identity to SEQ ID NO: 82, and a VL comprising an amino acid sequence having at least about 80% (e.g., at least about any one of 85%, 90%, 95%, 98%, 99%, or more) sequence identity to SEQ ID NO: 83. In some embodiments, the CD22 binding moiety comprises a VH comprising SEQ ID NO: 82 and a VL comprising SEQ ID NO: 83.

### Structure of the Recognition Molecules

The recognition molecules can be any binding moiety-containing molecules present on the surface of the engineered immune cell. The engineered immune cell in some embodiments comprises one or more nucleic acids that encode the recognition molecule or a portion thereof. The discussion in this section applies to both distal portion-recognition molecules and proximal portion-recognition molecules.

In some embodiments, the recognition molecule is an immune cell receptor, such as an immune cell receptor comprising an extracellular domain comprising a binding moiety (such binding moieties described in the sections above), a transmembrane domain, and an intracellular signaling domain. In some embodiments, the binding moiety in the extracellular domain is fused to the transmembrane domain directly or indirectly. For example, the recognition molecule (also referred to as immune cell receptor in this context) can be a single polypeptide that comprises, from N-terminus to the C-terminus: the binding moiety, an optional linker (e.g., a hinge sequence or an extracellular domain of a TCR subunit), the transmembrane domain, an optional linker (e.g., a co-stimulatory domain), and the intracellular signaling domain.

In some embodiments, the binding moiety in the extracellular domain is non-covalently bound to a polypeptide comprising the transmembrane domain. This can be accomplished, for example, by using two members of a binding pair, one fused to the binding moiety, the other fused to the transmembrane domain. The two components are brought together through interaction of the two members of the binding pair. For example, the recognition molecule (also referred to as immune cell receptor in this context) can comprise an extracellular domain comprising: i) a first polypeptide comprising the binding moiety and a first member of a binding pair; and ii) a second polypeptide comprising a second member of the binding pair, wherein the first member and the second member bind to each other non-covalently, and wherein the second member of the binding pair is fused to the transmembrane domain directly or indirectly. Suitable binding pairs include, but are not limited to, leucine zipper, biotin/streptavidin, MIC ligand/iNKG2D etc. See Cell 173, 1426-1438, Oncoimmunology. 2018; 7(1): e1368604, U.S. Ser. No. 10/259,858B2. In some embodiments, the binding moiety is fused to a polypeptide comprising the transmembrane domain.

In some embodiments, the recognition molecule is monovalent, i.e., has one binding moiety. In some embodiments, the recognition molecule is multivalent, i.e., has more than one binding moieties.

The recognition molecules described herein can be monospecific. In some embodiments, the recognition molecule is multispecific. For example, in some embodiments, the extracellular domain of the recognition molecule comprises a second antigen binding moiety specifically recognizing a second antigen. The second antigen binding moiety can be, for example, an sdAb (e.g., VHH), an scFv, a Fab′, a (Fab′)₂, an Fv, or a peptide ligand. The binding moiety and the second antigen binding moiety are linked in tandem. In some embodiments, the binding moiety is N-terminal to the second antigen binding moiety. In some embodiments, the binding moiety is C-terminal to the second antigen binding moiety. In some embodiments, the binding moiety and the second antigen binding moiety are linked via a linker. In some embodiments, the second antigen binding moiety specifically binds to an antigen on the surface of a T cell, such as CCR5.

In some embodiments, the transmembrane domain of recognition molecule (referred to as the immune cell receptor in this context) comprises one or more transmembrane domains derived from, for example, CD28, CD3ε, CD3ζ, CD45, CD4, CD5, CD8, CD9, CD16, CD22, CD33, CD37, CD64, CD80, CD86, CD134, CD137, or CD154.

The intracellular signaling domain of the recognition molecule (referred to as immune cell receptor in this context) in some embodiments comprises a functional primary immune cell signaling sequences, which include, but are not limited to, those found in a protein selected from the group consisting of CD3ζ, FcRγ, FcRβ, CD3γ, CD3δ, CD3ε, CD5, CD22, CD79a, CD79b, and CD66d. A “functional” primary immune cell signaling sequence is a sequence that is capable of transducing an immune cell activation signal when operably coupled to an appropriate receptor. “Non-functional” primary immune cell signaling sequences, which may comprises fragments or variants of primary immune cell signaling sequences, are unable to transduce an immune cell activation signal. In some embodiments, the intracellular signaling domain lacks a functional primary immune cell signaling sequence. In some embodiments, the intracellular signaling domain lack any primary immune cell signaling sequence.

### CAR

In some embodiments, the recognition molecule (referred to as immune cell receptor in this context) is a chimeric antigen receptor (“CAR”). The discussion in this section applies to both distal portion-recognition molecules and proximal portion-recognition molecules.

In some embodiments, the transmembrane domain of the CAR is derived from a molecule selected from the group consisting of CD8α, CD4, CD28, 4-1BB, CD80, CD86, CD152 and PD1. In some embodiments, the transmembrane domain of the CAR is derived from CD8α. In some embodiments, the transmembrane domain of the CAR comprises an amino acid sequence having at least about 80% (e.g., at least about any one of 85%, 90%, 95%, 98%, 99%, or more) sequence identity to SEQ ID NO: 37. In some embodiments, the transmembrane domain of the CAR has the amino acid sequence of SEQ ID NO: 37.

In some embodiments, the intracellular signaling domain of the CAR comprises a primary intracellular signaling domain derived from CD3ζ, FcRγ, FcRβ, CD3γ, CD3δ, CD3ε, CD5, CD22, CD79a, CD79b, or CD66d. In some embodiments, the primary intracellular signaling domain of the CAR is derived from CD3ζ. In some embodiments, the primary intracellular signaling domain of the CAR comprises an amino acid sequence having at least about 80% (e.g., at least about any one of 85%, 90%, 95%, 98%, 99%, or more) sequence identity to SEQ ID NO: 39. In some embodiments, the primary intracellular signaling domain of CAR has the sequence of SEQ ID NO: 39.

In some embodiments, the intracellular signaling domain of the CAR further comprises a co-stimulatory signaling domain. In some embodiments, the co-stimulatory signaling domain of the CAR is derived from a co-stimulatory molecule selected from the group consisting of CD27, CD28, 4-1BB, OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, DAP10, DAP12, CD83, ligands of CD83 and combinations thereof. In some embodiments, the co-stimulatory signaling domain of the CAR comprises a cytoplasmic domain of 4-1BB. In some embodiments, the co-stimulatory signaling domain of the CAR comprises an amino acid sequence having at least about 80% (e.g., at least about any one of 85%, 90%, 95%, 98%, 99%, or more) sequence identity to SEQ ID NO: 38. In some embodiments, the co-stimulatory signaling domain of the CAR has the sequence of SEQ ID NO: 38.

In some embodiments, the CAR further comprises a hinge domain located between the C-terminus of the extracellular domain and the N-terminus of the transmembrane domain. In some embodiments, the hinge domain is derived from CD8α. In some embodiments, the hinge domain is derived from an immunoglobulin (e.g., IgG1, IgG2, IgG3, IgG4, and IgD, for example, IgG4 CH2-CH3. In some embodiments, the hinge domain comprises an amino acid sequence having at least about 80% (e.g., at least about any one of 85%, 90%, 95%, 98%, 99%, or more) sequence identity to SEQ ID NO: 40. In some embodiments, the hinge domain has the amino acid sequence of 40.

In some embodiments, there is provided a CAR or a polypeptide comprising an amino acid sequence having at least about 80% (e.g., at least about any one of 85%, 90%, 95%, 98%, 99%, or more) sequence identity to SEQ ID NO: 75 or 84. In some embodiment, there is provided a CAR or a polypeptide comprising SEQ ID NO: 75 or 84.

cTCR

In some embodiments, the immune cell receptor is a chimeric T cell receptor (“cTCR”). The discussion in this section applies to both distal portion-recognition molecules and proximal portion-recognition molecules.

In some embodiments, the immune cell receptor described herein is a chimeric TCR receptor (“cTCR”). cTCRs typically comprise a chimeric receptor (CR) antigen binding domain linked (e.g., fused) directly or indirectly to the full-length or a portion of a TCR subunit, such as TCRα, TCRβ, TCRγ, TCRS, CD3γ, CD3ε, and CD3δ. The fusion polypeptide can be incorporated into a functional TCR complex along with other TCR subunits and confers antigen specificity to the TCR complex. In some embodiments, the binding domain is linked (e.g., fused) directly or indirectly to the full-length or a portion of the CD3F subunit (referred to as “eTCR”). The intracellular signaling domain of the cTCR can be derived from the intracellular signaling domain of a TCR subunit. The transmembrane domain of the cTCR can also be derived from a TCR subunit. In some embodiments, the intracellular signaling domain and the transmembrane domain of the cTCR are derived from the same TCR subunit. In some embodiments, the intracellular signaling domain and the transmembrane domain of the cTCR are derived from CD3ε. In some embodiments, the binding domain and the TCR subunit (or a portion thereof) can be fused via a linker (such as a GS linker). In some embodiments, the cTCR further comprises an extracellular domain of a TCR subunit or a portion thereof, which can be the same or different from the TCR subunit from which the intracellular signaling domain and/or transmembrane domain are derived from.

In some embodiments, the transmembrane domain of the cTCR is derived from the transmembrane domain of a TCR subunit selected from the group consisting of TCRα, TCRβ, TCRγ, TCRδ, CD3γ, CD3ε, and CD3δ. In some embodiments, the transmembrane domain of the cTCR is derived from the transmembrane domain of CD3. In some embodiments, the transmembrane domain of the cTCR comprises an amino acid sequence having at least about 80% (e.g., at least about any one of 85%, 90%, 95%, 98%, 99%, or more) sequence identity to SEQ ID NO: 41. In some embodiments, the transmembrane domain of the cTCR has the sequence of SEQ ID NO: 41.

In some embodiments, the intracellular signaling domain of the cTCR is derived from the intracellular signaling domain of a TCR subunit selected from the group consisting of TCRα, TCRβ, TCRγ, TCRδ, CD3γ, CD3ε, and CD3δ. In some embodiments, the intracellular signaling domain of the cTCR is derived from the intracellular signaling domain of CD3. In some embodiments, the intracellular signaling domain of the cTCR comprises an amino acid sequence having at least about 80% (e.g., at least about any one of 85%, 90%, 95%, 98%, 99%, or more) sequence identity to SEQ ID NO: 42. In some embodiments, the intracellular signaling domain of the cTCR has the sequence of SEQ ID NO: 42.

In some embodiments, the transmembrane domain and intracellular signaling domain of the cTCR are derived from the same TCR subunit. In some embodiments, the cTCR further comprises at least a portion of an extracellular sequence of a TCR subunit, and the TCR extracellular sequence in some embodiments may be derived from the same TCR subunit as the transmembrane domain and/or intracellular signaling domain. In some embodiments, the cTCR comprises a full-length TCR subunit. For example, in some embodiments, the cTCR comprises a binding domain fused (directly or indirectly) to the N-terminus of a TCR subunit (e.g., CD3ε).

### Binding Moieties

The binding moieties described herein can be an antibody moiety or a ligand that specifically recognizing a portion in the extracellular domain of a target molecule. The discussion in this section applies to both distal portion-recognition molecules and proximal portion-recognition molecules.

In some embodiments, the binding moiety specifically binds the target molecule with a) an affinity that is at least about 10 (including for example at least about any of 10, 20, 30, 40, 50, 75, 100, 200, 300, 400, 500, 750, 1000 or more) times its binding affinity for other molecules; or b) a K_{d} no more than about 1/10 (such as no more than about any of 1/10, 1/20, 1/30, 1/40, 1/50, 1/75, 1/100, 1/200, 1/300, 1/400, 1/500, 1/750, 1/1000 or less) times its K_{d} for binding to other molecules. Binding affinity can be determined by methods known in the art, such as ELISA, fluorescence activated cell sorting (FACS) analysis, or radioimmunoprecipitation assay (RIA). K_{d} can be determined by methods known in the art, such as surface plasmon resonance (SPR) assay utilizing, for example, Biacore instruments, or kinetic exclusion assay (KinExA) utilizing, for example, Sapidyne instruments.

In some embodiments, the binding moiety is selected from the group consisting of Fab, a Fab′, a (Fab′)₂, an Fv, a single chain Fv (scFv), a single domain antibody (sdAb), and a peptide ligand specifically binding to the target molecule.

In some embodiments, the binding moiety is an antibody moiety. In some embodiments, the antibody moiety is monospecific. In some embodiments, the antibody moiety is multi-specific. In some embodiments, the antibody moiety is bispecific. In some embodiments, the antibody moiety is a tandem scFv, a diabody (db), a single chain diabody (scDb), a dual-affinity retargeting (DART) antibody, a dual variable domain (DVD) antibody, a chemically cross-linked antibody, a heteromultimeric antibody, or a heteroconjugate antibody. In some embodiments, the antibody moiety is a scFv. In some embodiments, the antibody moiety is a single domain antibody (sdAb). In some embodiments, the antibody moiety is a VHH. In some embodiments, the antibody moiety is fully human, semi-synthetic with human antibody framework regions, or humanized.

The antibody moiety in some embodiments comprises specific CDR sequences derived from one or more antibody moieties (such as any of the reference antibodies disclosed herein) or certain variants of such sequences comprising one or more amino acid substitutions. In some embodiments, the amino acid substitutions in the variant sequences do not substantially reduce the ability of the antigen-binding domain to bind the target antigen. Alterations that substantially improve target antigen binding affinity or affect some other property, such as specificity and/or cross-reactivity with related variants of the target antigen, are also contemplated.

In some embodiments, the binding moiety binds to target molecule with a K_{d} between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any values and ranges between these values).

### Exemplary Anti-CD22 Immune Cell Receptors

In some embodiments, there is provided an anti-CD22 D1-4 immune cell receptor comprising: i) an extracellular domain comprising a CD22 binding moiety that specifically binds to an epitope within D1-4 of CD22; ii) a transmembrane domain, and iii) an intracellular signaling domain. In some embodiments, there is provided an engineered immune cell comprising: an anti-CD22 D1-4 immune cell receptor comprising: i) an extracellular domain comprising a CD22 binding moiety that specifically binds to an epitope within D1-4 of CD22; ii) a transmembrane domain, and iii) an intracellular signaling domain. In some embodiments, there is provided an engineered immune cell comprising: one or more nucleic acids encoding an anti-CD22 D1-4 immune cell receptor, wherein the anti-CD22 immune cell receptor comprises: i) an extracellular domain comprising a CD22 binding moiety that specifically binds to an epitope within D1-4 of CD22; ii) a transmembrane domain, and iii) an intracellular signaling domain. In some embodiments, the engineered immune cell further comprises one or more co-receptors (such as a cytokine receptor) or one or more nucleic acids encoding one or more co-receptors (such as a cytokine receptor).

In some embodiments, there is provided an anti-CD22 D5-7 immune cell receptor comprising: i) an extracellular domain comprising a CD22 binding moiety that specifically binds to an epitope within D5-7 of CD22; ii) a transmembrane domain, and iii) an intracellular signaling domain. In some embodiments, there is provided an engineered immune cell comprising: an anti-CD22 D5-7 immune cell receptor comprising: i) an extracellular domain comprising a CD22 binding moiety that specifically binds to an epitope within D5-7 of CD22; ii) a transmembrane domain, and iii) an intracellular signaling domain. In some embodiments, there is provided an engineered immune cell comprising: one or more nucleic acids encoding an anti-CD22 immune cell receptor, wherein the anti-CD22 D5-7 immune cell receptor comprises: i) an extracellular domain comprising a CD22 binding moiety that specifically binds to an epitope within D5-7 of CD22; ii) a transmembrane domain, and iii) an intracellular signaling domain. In some embodiments, the engineered immune cell further comprises one or more co-receptors (such as a cytokine receptor) or one or more nucleic acids encoding one or more co-receptors (such as a cytokine receptor).

In some embodiments, the anti-CD22 immune cell receptor described herein is a chimeric antigen receptor (“CAR”). Thus, for example, in some embodiments, there is provided an anti-CD22 D1-4 CAR comprising: i) an extracellular domain comprising a CD22 binding moiety that specifically binds to an epitope within D1-4 of CD22 (for example an anti-CD22 antibody moiety such as scFv or sdAb); ii) an optional hinge sequence (such as a hinge sequence derived from CD8); iii) a transmembrane domain (such as a CD8 transmembrane domain), iv) an intracellular co-stimulatory domain (such as a co-stimulatory domain derived from 4-1BB or CD28); and v) an intracellular signaling domain (such as an intracellular signaling domain derived from CD3ζ). In some embodiments, there is provided an engineered immune cell comprising an anti-CD22 D1-4 CAR comprising: i) an extracellular domain comprising a CD22 binding moiety that specifically binds to an epitope within D1-4 of CD22 (for example an anti-CD4 antibody moiety such as scFv or sdAb); ii) an optional hinge sequence (such as a hinge sequence derived from CD8); iii) a transmembrane domain (such as a CD8 transmembrane domain), iv) an intracellular co-stimulatory domain (such as a co-stimulatory domain derived from 4-1BB or CD28); and v) an intracellular signaling domain (such as an intracellular signaling domain derived from CD3ζ). In some embodiments, there is provided an engineered immune cell comprising: one or more nucleic acids encoding an anti-CD22 D1-4 CAR comprising: i) an extracellular domain comprising a CD22 binding moiety that specifically binds to an epitope within D1-4 of CD22 (for example an anti-CD22 antibody moiety such as scFv or sdAb); ii) an optional hinge sequence (such as a hinge sequence derived from CD8); iii) a transmembrane domain (such as a CD8 transmembrane domain), iv) an intracellular co-stimulatory domain (such as a co-stimulatory domain derived from 4-1BB or CD28); and v) an intracellular signaling domain (such as an intracellular signaling domain derived from CD3ζ). In some embodiments, the engineered immune cell further comprises one or more co-receptors (such as a cytokine receptor) or one or more nucleic acids encoding one or more co-receptors (such as a cytokine receptor).

In some embodiments, there is provided an anti-CD22 D5-7 CAR comprising: i) an extracellular domain comprising a CD22 binding moiety that specifically binds to an epitope within D5-7 of CD22 (for example an anti-CD22 D5-7 antibody moiety such as scFv or sdAb); ii) an optional hinge sequence (such as a hinge sequence derived from CD8); iii) a transmembrane domain (such as a CD8 transmembrane domain), iv) an intracellular co-stimulatory domain (such as a co-stimulatory domain derived from 4-1BB or CD28); and v) an intracellular signaling domain (such as an intracellular signaling domain derived from CD3ζ). In some embodiments, there is provided an engineered immune cell comprising an anti-CD22 D5-7 CAR comprising: i) an extracellular domain comprising a CD22 binding moiety that specifically binds to an epitope within D5-7 of CD22 (for example an anti-CD22 D5-7 antibody moiety such as scFv or sdAb); ii) an optional hinge sequence (such as a hinge sequence derived from CD8); iii) a transmembrane domain (such as a CD8 transmembrane domain), iv) an intracellular co-stimulatory domain (such as a co-stimulatory domain derived from 4-1BB or CD28); and v) an intracellular signaling domain (such as an intracellular signaling domain derived from CD3ζ). In some embodiments, there is provided an engineered immune cell comprising: one or more nucleic acids encoding an anti-CD22 D5-7 CAR comprising: i) an extracellular domain comprising a CD22 binding moiety that specifically binds to an epitope within D5-7 of CD22 (for example an anti-CD22 D5-7 antibody moiety such as scFv or sdAb); ii) an optional hinge sequence (such as a hinge sequence derived from CD8); iii) a transmembrane domain (such as a CD8 transmembrane domain), iv) an intracellular co-stimulatory domain (such as a co-stimulatory domain derived from 4-1BB or CD28); and v) an intracellular signaling domain (such as an intracellular signaling domain derived from CD3ζ). In some embodiments, the engineered immune cell further comprises one or more co-receptors (such as a cytokine receptor) or one or more nucleic acids encoding one or more co-receptors (such as a cytokine receptor).

In some embodiments, the anti-CD4 immune cell receptor is a chimeric T cell receptor (“cTCR.”). In some embodiments, there is provided an anti-CD22 D1-4 cTCR comprising: i) an extracellular domain comprising a CD22 binding moiety that specifically binds to an epitope within D1-4 of CD22 (for example an anti-CD22 D1-4 antibody moiety such as scFv or sdAb); ii) an optional linker (such as a GS linker); iii) an optional extracellular domain of a TCR subunit or a portion thereof, iii) a transmembrane domain derived from a TCR subunit, and iv) an intracellular signaling domain derived from a TCR subunit. In some embodiments, there is provided an engineered immune cell comprising an anti-CD22 D1-4 cTCR comprising: i) an extracellular domain comprising a CD22 binding moiety that specifically binds to an epitope within D1-4 of CD22 (for example an anti-CD22 D1-4 antibody moiety such as scFv or sdAb); ii) an optional linker (such as a GS linker); iii) an optional extracellular domain of a TCR subunit or a portion thereof, iii) a transmembrane domain derived from a TCR subunit, and iv) an intracellular signaling domain derived from a TCR subunit. In some embodiments, there is provided an engineered immune cell comprising: one or more nucleic acids encoding an anti-CD22 D1-4 cTCR comprising: i) an extracellular domain comprising a CD22 binding moiety that specifically binds to an epitope within D1-4 of CD22 (for example an anti-CD22 D1-4 antibody moiety such as scFv or sdAb); ii) an optional linker (such as a GS linker); iii) an optional extracellular domain of a TCR subunit or a portion thereof, iii) a transmembrane domain derived from a TCR subunit, and iv) an intracellular signaling domain derived from a TCR subunit. In some embodiments, the TCR subunit is selected from the group consisting of TCRα, TCRβ, TCRγ, TCRδ, CD3γ, and CD3ε. In some embodiments, the transmembrane domain, the intracellular signaling domain, and the optional extracellular domain of a TCR subunit or a portion thereof are derived from the same TCR subunit. In some embodiments, the transmembrane domain, the intracellular signaling domain, and the optional extracellular domain of a TCR subunit or a portion thereof are derived from CD3ε. In some embodiments, the anti-CD22 D1-4 cTCR comprises the CD22 binding domain fused to the N-terminus of a full length CD3ε. In some embodiments, the engineered immune cell further comprises one or more co-receptors (such as a cytokine receptor) or one or more nucleic acids encoding one or more co-receptors (such as a cytokine receptor).

In some embodiments, there is provided an anti-CD22 D5-7 cTCR comprising: i) an extracellular domain comprising: a CD22 binding moiety that specifically binds to an epitope within D5-7 of CD22 (for example an anti-CD22 D5-7 antibody moiety such as scFv or sdAb); ii) an optional linker (such as a GS linker); iii) an optional extracellular domain of a TCR subunit or a portion thereof, iii) a transmembrane domain derived from a TCR subunit, and iv) an intracellular signaling domain derived from a TCR subunit. In some embodiments, there is provided an engineered immune cell comprising an anti-CD22 D5-7 cTCR comprising: i) an extracellular domain comprising a CD22 binding moiety that specifically binds to an epitope within D5-7 of CD22 (for example an anti-CD22 D5-7 antibody moiety such as scFv or sdAb); ii) an optional linker (such as a GS linker); iii) an optional extracellular domain of a TCR subunit or a portion thereof, iii) a transmembrane domain derived from a TCR subunit, and iv) an intracellular signaling domain derived from a TCR subunit. In some embodiments, there is provided an engineered immune cell comprising: one or more nucleic acids encoding an anti-CD22 D5-7 cTCR comprising: i) an extracellular domain comprising a CD22 binding moiety that specifically binds to an epitope within D5-7 of CD22 (for example an anti-CD22 D5-7 antibody moiety such as scFv or sdAb); ii) an optional linker (such as a GS linker); iii) an optional extracellular domain of a TCR subunit or a portion thereof; iii) a transmembrane domain derived from a TCR subunit, and iv) an intracellular signaling domain derived from a TCR subunit. In some embodiments, the TCR subunit is selected from the group consisting of TCRα, TCRβ, TCRγ, TCRδ, CD3γ, and CD3ε. In some embodiments, the transmembrane domain, the intracellular signaling domain, and the optional extracellular domain of a TCR subunit or a portion thereof are derived from the same TCR subunit. In some embodiments, the transmembrane domain, the intracellular signaling domain, and the optional extracellular domain of a TCR subunit or a portion thereof are derived from CD3ε. In some embodiments, the anti-CD22 D5-7 cTCR comprises the CD22 binding domain fused to the N-terminus of a full length CD3ε. In some embodiments, the engineered immune cell further comprises one or more co-receptors (such as a cytokine receptor) or one or more nucleic acids encoding one or more co-receptors (such as a cytokine receptor).

### Engineered Immune Cells

In some embodiments, there is provided an engineered immune cell (such as cytotoxic T cell, NK cell, or γδT cell) comprising on its surface a recognition molecule that comprises a binding moiety specifically binding to a target molecule on the surface of a target cell, wherein the target molecule comprises an extracellular domain (such as an extracellular domain that is at least about 175 amino acids long), wherein the binding moiety specifically binds to a distal portion of the extracellular domain, wherein the immune cell is capable of killing a target cell that comprises on its surface the target molecule, and wherein the immune cell is capable of killing a target cell that comprises on its surface both the target molecule and the recognition molecule. In some embodiments, the binding moiety binds to a region (e.g. an epitope) in the extracellular domain that is about 50 amino acids or more (such as about any of 60, 70, 80, 90, 100, 110, 120, 130, 140, 150, 160, 170, 180, 190, or 200 amino acids or more) away from the C-terminus of the extracellular domain. In some embodiments, the binding moiety binds to a region (e.g. an epitope) that is within about 120 amino acids (such as within about any of 110, 100, 90, 80, 70, 60, 50, 40, or 30 amino acids) from the N-terminus of the extracellular domain. In some embodiments, the target molecule is a transmembrane receptor, such as a transmembrane receptor selected from the group consisting of CD22, CD4, CD21 (CR2), CD30, ROR1, CD5, and CD20. In some embodiments, the target molecule is CD4. In some embodiments, the target molecule is CD22.

In some embodiments, there is provided an engineered immune cell (such as cytotoxic T cell, NK cell, or γδT cell) comprising on its surface a recognition molecule that comprises a binding moiety specifically binding to a target molecule on the surface of a target cell, wherein the target molecule comprises an extracellular domain comprising three or more Ig-like domains, wherein the binding moiety specifically binds to a distal portion of the extracellular domain, wherein the immune cell is capable of killing a target cell that comprises on its surface the target molecule, and wherein the immune cell is capable of killing a target cell that comprises on its surface both the target molecule and the recognition molecule. In some embodiments, the binding moiety binds to a region outside the first two Ig-like domains from the C-terminal end of the extracellular domain. In some embodiments, the binding moiety binds to a region within the first Ig-like domain at the N-terminal end of the extracellular domain. In some embodiments, the target molecule is a transmembrane receptor, such as a transmembrane receptor selected from the group consisting of CD22, CD4, CD21 (CR2), CD30, ROR1, CD5, and CD20. In some embodiments, the target molecule is CD4. In some embodiments, the target molecule is CD22.

In some embodiments, there is provided an engineered immune cell (such as cytotoxic T cell, NK cell, or γδT cell) comprising on its surface an immune cell receptor that comprises a binding moiety specifically binding to a target molecule on the surface of a target cell, a transmembrane domain, and an intracellular signaling domain, wherein the target molecule comprises an extracellular domain (such as an extracellular domain that is at least about 175 amino acids long), wherein the binding moiety specifically binds to a distal portion of the extracellular domain, wherein the immune cell is capable of killing a target cell that comprises on its surface the target molecule, and wherein the immune cell is capable of killing a target cell that comprises on its surface both the target molecule and the immune cell receptor. In some embodiments, the binding moiety binds to a region (e.g. an epitope) in the extracellular domain that is about 50 amino acids or more (such as about any of 60, 70, 80, 90, 100, 110, 120, 130, 140, 150, 160, 170, 180, 190, or 200 amino acids or more) away from the C-terminus of the extracellular domain. In some embodiments, the binding moiety binds to a region (e.g. an epitope) that is within about 120 amino acids (such as within about any of 110, 100, 90, 80, 70, 60, 50, 40, or 30 amino acids) from the N-terminus of the extracellular domain. In some embodiments, the target molecule is a transmembrane receptor, such as a transmembrane receptor selected from the group consisting of CD22, CD4, CD21 (CR2), CD30, ROR1, CD5, and CD20. In some embodiments, the target molecule is CD4. In some embodiments, the target molecule is CD22.

In some embodiments, there is provided an engineered immune cell (such as cytotoxic T cell, NK cell, or γδT cell) comprising on its surface an immune cell receptor that comprises a binding moiety specifically binding to a target molecule on the surface of a target cell, a transmembrane domain, and an intracellular signaling domain, wherein the target molecule comprises an extracellular domain comprising three or more Ig-like domains, wherein the binding moiety specifically binds to a distal portion of the extracellular domain, wherein the immune cell is capable of killing a target cell that comprises on its surface the target molecule, and wherein the immune cell is capable of killing a target cell that comprises on its surface both the target molecule and the immune cell receptor. In some embodiments, the binding moiety binds to a region outside the first two Ig-like domains from the C-terminal end of the extracellular domain. In some embodiments, the binding moiety binds to a region within the first Ig-like domain at the N-terminal end of the extracellular domain. In some embodiments, the target molecule is a transmembrane receptor, such as a transmembrane receptor selected from the group consisting of CD22, CD4, CD21 (CR2), CD30, ROR1, CD5, and CD20. In some embodiments, the target molecule is CD4. In some embodiments, the target molecule is CD22.

In some embodiments, there is provided an engineered immune cell (such as cytotoxic T cell, NK cell, or γδT cell) comprising on its surface a chimeric antigen receptor (CAR) that comprises a binding moiety specifically binding to a target molecule on the surface of a target cell, a transmembrane domain, and an intracellular signaling domain, wherein the target molecule comprises an extracellular domain (such as an extracellular domain that is at least about 175 amino acids long), wherein the binding moiety specifically binds to a distal portion of the extracellular domain, wherein the immune cell is capable of killing a target cell that comprises on its surface the target molecule, and wherein the immune cell is capable of killing a target cell that comprises on its surface both the target molecule and the CAR. In some embodiments, the CAR comprises: i) an extracellular domain comprising the binding moiety; ii) an optional hinge sequence (such as a hinge sequence derived from CD8); iii) a transmembrane domain (such as a CD8 transmembrane domain), iv) an intracellular co-stimulatory domain (such as a co-stimulatory domain derived from 4-1BB or CD28); and v) an intracellular signaling domain (such as an intracellular signaling domain derived from CD3ζ). In some embodiments, the binding moiety binds to a region (e.g. an epitope) in the extracellular domain that is about 50 amino acids or more (such as about any of 60, 70, 80, 90, 100, 110, 120, 130, 140, 150, 160, 170, 180, 190, or 200 amino acids or more) away from the C-terminus of the extracellular domain. In some embodiments, the binding moiety binds to a region (e.g. an epitope) that is within about 120 amino acids (such as within about any of 110, 100, 90, 80, 70, 60, 50, 40, or 30 amino acids) from the N-terminus of the extracellular domain. In some embodiments, the target molecule is a transmembrane receptor, such as a transmembrane receptor selected from the group consisting of CD22, CD4, CD21 (CR2), CD30, ROR1, CD5, and CD20. In some embodiments, the target molecule is CD4. In some embodiments, the target molecule is CD22.

In some embodiments, there is provided an engineered immune cell (such as cytotoxic T cell, NK cell, or γδT cell) comprising on its surface a chimeric antigen receptor (CAR) that comprises a binding moiety specifically binding to a target molecule on the surface of a target cell, a transmembrane domain, and an intracellular signaling domain, wherein the target molecule comprises an extracellular domain comprising three or more Ig-like domains, wherein the binding moiety specifically binds to a distal portion of the extracellular domain, wherein the immune cell is capable of killing a target cell that comprises on its surface the target molecule, and wherein the immune cell is capable of killing a target cell that comprises on its surface both the target molecule and the CAR. In some embodiments, the CAR comprises: i) an extracellular domain comprising the binding moiety; ii) an optional hinge sequence (such as a hinge sequence derived from CD8); iii) a transmembrane domain (such as a CD8 transmembrane domain), iv) an intracellular co-stimulatory domain (such as a co-stimulatory domain derived from 4-1BB or CD28); and v) an intracellular signaling domain (such as an intracellular signaling domain derived from CD3ζ). In some embodiments, the binding moiety binds to a region outside the first two Ig-like domains from the C-terminal end of the extracellular domain. In some embodiments, the binding moiety binds to a region within the first Ig-like domain at the N-terminal end of the extracellular domain. In some embodiments, the target molecule is a transmembrane receptor, such as a transmembrane receptor selected from the group consisting of CD22, CD4, CD21 (CR2), CD30, ROR1, CD5, and CD20. In some embodiments, the target molecule is CD4. In some embodiments, the target molecule is CD22.

In some embodiments, there is provided an engineered immune cell (such as cytotoxic T cell, NK cell, or γδT cell) comprising on its surface a chimeric T cell receptor (cTCR) that comprises a binding moiety specifically binding to a target molecule on the surface of a target cell, a transmembrane domain, and an intracellular signaling domain, wherein the target molecule comprises an extracellular domain (such as an extracellular domain that is at least about 175 amino acids long), wherein the binding moiety specifically binds to a distal portion of the extracellular domain, wherein the immune cell is capable of killing a target cell that comprises on its surface the target molecule, and wherein the immune cell is capable of killing a target cell that comprises on its surface both the target molecule and the cTCR. In some embodiments, the cTCR comprises: i) an extracellular domain comprising the binding moiety; ii) an optional linker (such as a GS linker); iii) an optional extracellular domain of a TCR subunit or a portion thereof, iii) a transmembrane domain derived from a TCR subunit, and iv) an intracellular signaling domain derived from a TCR subunit. In some embodiments, the TCR subunit is selected from the group consisting of TCRα, TCRβ, TCRγ, TCRδ, CD3γ, and CD3ε. In some embodiments, the transmembrane domain, the intracellular signaling domain, and the optional extracellular domain of a TCR subunit or a portion thereof are derived from the same TCR subunit. In some embodiments, the transmembrane domain, the intracellular signaling domain, and the optional extracellular domain of a TCR subunit or a portion thereof are derived from CD3ε. In some embodiments, the cTCR comprises the binding moiety fused to the N-terminus of a full length CD3ε. In some embodiments, the binding moiety binds to a region (e.g. an epitope) in the extracellular domain that is about 50 amino acids or more (such as about any of 60, 70, 80, 90, 100, 110, 120, 130, 140, 150, 160, 170, 180, 190, or 200 amino acids or more) away from the C-terminus of the extracellular domain. In some embodiments, the binding moiety binds to a region (e.g. an epitope) that is within about 120 amino acids (such as within about any of 110, 100, 90, 80, 70, 60, 50, 40, or 30 amino acids) from the N-terminus of the extracellular domain. In some embodiments, the target molecule is a transmembrane receptor, such as a transmembrane receptor selected from the group consisting of CD22, CD4, CD21 (CR2), CD30, ROR1, CD5, and CD20. In some embodiments, the target molecule is CD4. In some embodiments, the target molecule is CD22.

In some embodiments, there is provided an engineered immune cell (such as cytotoxic T cell, NK cell, or γδT cell) comprising on its surface a chimeric T cell receptor (cTCR) that comprises a binding moiety specifically binding to a target molecule on the surface of a target cell, a transmembrane domain, and an intracellular signaling domain, wherein the target molecule comprises an extracellular domain comprising three or more Ig-like domains, wherein the binding moiety specifically binds to a distal portion of the extracellular domain, wherein the immune cell is capable of killing a target cell that comprises on its surface the target molecule, and wherein the immune cell is capable of killing a target cell that comprises on its surface both the target molecule and the cTCR. In some embodiments, the cTCR comprises: i) an extracellular domain comprising the binding moiety; ii) an optional linker (such as a GS linker); iii) an optional extracellular domain of a TCR subunit or a portion thereof; iii) a transmembrane domain derived from a TCR subunit, and iv) an intracellular signaling domain derived from a TCR subunit. In some embodiments, the TCR subunit is selected from the group consisting of TCRα, TCRβ, TCRγ, TCRδ, CD3γ, and CD3ε. In some embodiments, the transmembrane domain, the intracellular signaling domain, and the optional extracellular domain of a TCR subunit or a portion thereof are derived from the same TCR subunit. In some embodiments, the transmembrane domain, the intracellular signaling domain, and the optional extracellular domain of a TCR subunit or a portion thereof are derived from CD3ε. In some embodiments, the cTCR comprises the binding moiety fused to the N-terminus of a full length CD3ε. In some embodiments, the binding moiety binds to a region outside the first two Ig-like domains from the C-terminal end of the extracellular domain. In some embodiments, the binding moiety binds to a region within the first Ig-like domain at the N-terminal end of the extracellular domain. In some embodiments, the target molecule is a transmembrane receptor, such as a transmembrane receptor selected from the group consisting of CD22, CD4, CD21 (CR2), CD30, ROR1, CD5, and CD20. In some embodiments, the target molecule is CD4. In some embodiments, the target molecule is CD22.

In another aspect, there is provided an engineered immune cell (such as cytotoxic T cell, NK cell, or γδT cell) comprising on its surface a recognition molecule that comprises a binding moiety specifically binding to a target molecule on the surface of a target cell, wherein the target molecule comprises an extracellular domain (such as an extracellular domain that is at least about 175 amino acids long), wherein the binding moiety specifically binds to a proximal portion of the extracellular domain, wherein the engineered immune cell is capable of killing a target cell that comprises on its surface the target molecule, and wherein the engineered immune cell has no or reduced capability of killing a target cell comprising on its surface both the target molecule and the recognition molecule. In some embodiments, the binding moiety binds to a region (e.g. epitope) in the extracellular domain that is outside a region that is about 80 amino acids or more (such as about any of 90, 100, 110, 120 amino acids or more) away from the N-terminus of the extracellular domain. In some embodiments, the binding moiety binds to a region (e.g. epitope) in the extracellular domain that is within about 102 amino acids (e.g. within about any of 100, 90, 80, 70, 60, 50, 40, or 30 amino acids) from the C-terminus of the extracellular domain. In some embodiments, the target molecule is a transmembrane receptor, such as a transmembrane receptor selected from the group consisting of CD22, CD4, CD21 (CR2), CD30, ROR1, CD5, and CD20. In some embodiments, the target molecule is CD4. In some embodiments, the target molecule is CD22.

In some embodiments, there is provided an engineered immune cell (such as cytotoxic T cell, NK cell, or γδT cell) comprising on its surface a recognition molecule that comprises a binding moiety specifically binding to a target molecule on the surface of a target cell, wherein the target molecule comprises an extracellular domain comprising two or more Ig-like domains, wherein the binding moiety specifically binds to a proximal portion of the extracellular domain, wherein the engineered immune cell is capable of killing a target cell that comprises on its surface the target molecule, and wherein the engineered immune cell has no or reduced capability of killing a target cell comprising on its surface both the target molecule and the recognition molecule. In some embodiments, the binding moiety binds to a region (e.g. epitope) outside the first Ig-like domain at the N-terminal end of the extracellular domain. In some embodiments, the binding moiety binds to a region (e.g. epitope) within the first two Ig-like domains from the C-terminal end of the extracellular domain. In some embodiments, the target molecule is a transmembrane receptor, such as a transmembrane receptor selected from the group consisting of CD22, CD4, CD21 (CR2), CD30, ROR1, CD5, and CD20. In some embodiments, the target molecule is CD4. In some embodiments, the target molecule is CD22.

In some embodiments, there is provided an engineered immune cell (such as cytotoxic T cell, NK cell, or γδT cell) comprising on its surface an immune cell receptor that comprises a binding moiety specifically binding to a target molecule on the surface of a target cell, a transmembrane domain, and an intracellular signaling domain, wherein the target molecule comprises an extracellular domain (such as an extracellular domain that is at least about 175 amino acids long), wherein the binding moiety specifically binds to a proximal portion of the extracellular domain, wherein the engineered immune cell is capable of killing a target cell that comprises on its surface the target molecule, and wherein the engineered immune cell has no or reduced capability of killing a target cell comprising on its surface both the target molecule and the immune cell receptor. In some embodiments, the binding moiety binds to a region (e.g. epitope) in the extracellular domain that is outside a region that is about 80 amino acids or more (such as about any of 90, 100, 110, 120 amino acids or more) away from the N-terminus of the extracellular domain. In some embodiments, the binding moiety binds to a region (e.g. epitope) in the extracellular domain that is within about 102 amino acids (e.g. within about any of 100, 90, 80, 70, 60, 50, 40, or 30 amino acids) from the C-terminus of the extracellular domain. In some embodiments, the target molecule is a transmembrane receptor, such as a transmembrane receptor selected from the group consisting of CD22, CD4, CD21 (CR2), CD30, ROR1, CD5, and CD20. In some embodiments, the target molecule is CD4. In some embodiments, the target molecule is CD22.

In some embodiments, there is provided an engineered immune cell (such as cytotoxic T cell, NK cell, or γδT cell) comprising on its surface an immune cell receptor that comprises a binding moiety specifically binding to a target molecule on the surface of a target cell, a transmembrane domain, and an intracellular signaling domain, wherein the target molecule comprises an extracellular domain comprising two or more Ig-like domains, wherein the binding moiety specifically binds to a proximal portion of the extracellular domain, wherein the engineered immune cell is capable of killing a target cell that comprises on its surface the target molecule, and wherein the engineered immune cell has no or reduced capability of killing a target cell comprising on its surface both the target molecule and the immune cell receptor. In some embodiments, the binding moiety binds to a region (e.g. epitope) outside the first Ig-like domain at the N-terminal end of the extracellular domain. In some embodiments, the binding moiety binds to a region (e.g. epitope) within the first two Ig-like domains from the C-terminal end of the extracellular domain. In some embodiments, the target molecule is a transmembrane receptor, such as a transmembrane receptor selected from the group consisting of CD22, CD4, CD21 (CR2), CD30, ROR1, CD5, and CD20. In some embodiments, the target molecule is CD4. In some embodiments, the target molecule is CD22.

In some embodiments, there is provided an engineered immune cell (such as cytotoxic T cell, NK cell, or γδT cell) comprising on its surface a chimeric antigen receptor (CAR) that comprises a binding moiety specifically binding to a target molecule on the surface of a target cell, a transmembrane domain, and an intracellular signaling domain, wherein the target molecule comprises an extracellular domain (such as an extracellular domain that is at least about 175 amino acids long), wherein the binding moiety specifically binds to a proximal portion of the extracellular domain, wherein the engineered immune cell is capable of killing a target cell that comprises on its surface the target molecule, and wherein the engineered immune cell has no or reduced capability of killing a target cell comprising on its surface both the target molecule and the CAR. In some embodiments, the CAR comprises: i) an extracellular domain comprising the binding moiety; ii) an optional hinge sequence (such as a hinge sequence derived from CD8); iii) a transmembrane domain (such as a CD8 transmembrane domain), iv) an intracellular co-stimulatory domain (such as a co-stimulatory domain derived from 4-1BB or CD28); and v) an intracellular signaling domain (such as an intracellular signaling domain derived from CD3ζ). In some embodiments, the binding moiety binds to a region (e.g. epitope) in the extracellular domain that is outside a region that is about 80 amino acids or more (such as about any of 90, 100, 110, 120 amino acids or more) away from the N-terminus of the extracellular domain. In some embodiments, the binding moiety binds to a region (e.g. epitope) in the extracellular domain that is within about 102 amino acids (e.g. within about any of 100, 90, 80, 70, 60, 50, 40, or 30 amino acids) from the C-terminus of the extracellular domain. In some embodiments, the target molecule is a transmembrane receptor, such as a transmembrane receptor selected from the group consisting of CD22, CD4, CD21 (CR2), CD30, ROR1, CD5, and CD20. In some embodiments, the target molecule is CD4. In some embodiments, the target molecule is CD22.

In some embodiments, there is provided an engineered immune cell (such as cytotoxic T cell, NK cell, or γδT cell) comprising on its surface a chimeric antigen receptor (CAR) that comprises a binding moiety specifically binding to a target molecule on the surface of a target cell, a transmembrane domain, and an intracellular signaling domain, wherein the target molecule comprises an extracellular domain comprising two or more Ig-like domains, wherein the binding moiety specifically binds to a proximal portion of the extracellular domain, wherein the engineered immune cell is capable of killing a target cell that comprises on its surface the target molecule, and wherein the engineered immune cell has no or reduced capability of killing a target cell comprising on its surface both the target molecule and the CAR. In some embodiments, the CAR comprises: i) an extracellular domain comprising the binding moiety; ii) an optional hinge sequence (such as a hinge sequence derived from CD8); iii) a transmembrane domain (such as a CD8 transmembrane domain), iv) an intracellular co-stimulatory domain (such as a co-stimulatory domain derived from 4-1BB or CD28); and v) an intracellular signaling domain (such as an intracellular signaling domain derived from CD3ζ). In some embodiments, the binding moiety binds to a region (e.g. epitope) outside the first Ig-like domain at the N-terminal end of the extracellular domain. In some embodiments, the binding moiety binds to a region (e.g. epitope) within the first two Ig-like domains from the C-terminal end of the extracellular domain. In some embodiments, the target molecule is a transmembrane receptor, such as a transmembrane receptor selected from the group consisting of CD22, CD4, CD21 (CR2), CD30, ROR1, CD5, and CD20. In some embodiments, the target molecule is CD4. In some embodiments, the target molecule is CD22.

In some embodiments, there is provided an engineered immune cell (such as cytotoxic T cell, NK cell, or γδT cell) comprising on its surface a chimeric T cell receptor (cTCR) that comprises a binding moiety specifically binding to a target molecule on the surface of a target cell, a transmembrane domain, and an intracellular signaling domain, wherein the target molecule comprises an extracellular domain (such as an extracellular domain that is at least about 175 amino acids long), wherein the binding moiety specifically binds to a proximal portion of the extracellular domain, wherein the engineered immune cell is capable of killing a target cell that comprises on its surface the target molecule, and wherein the engineered immune cell has no or reduced capability of killing a target cell comprising on its surface both the target molecule and the cTCR. In some embodiments, the cTCR comprises: i) an extracellular domain comprising the binding moiety; ii) an optional linker (such as a GS linker); iii) an optional extracellular domain of a TCR subunit or a portion thereof; iii) a transmembrane domain derived from a TCR subunit, and iv) an intracellular signaling domain derived from a TCR subunit. In some embodiments, the TCR subunit is selected from the group consisting of TCRα, TCRβ, TCR7, TCRδ, CD3γ, and CD3ε. In some embodiments, the transmembrane domain, the intracellular signaling domain, and the optional extracellular domain of a TCR subunit or a portion thereof are derived from the same TCR subunit. In some embodiments, the transmembrane domain, the intracellular signaling domain, and the optional extracellular domain of a TCR subunit or a portion thereof are derived from CD3ε. In some embodiments, the cTCR comprises the binding moiety fused to the N-terminus of a full length CD3ε. In some embodiments, the binding moiety binds to a region (e.g. epitope) in the extracellular domain that is outside a region that is about 80 amino acids or more (such as about any of 90, 100, 110, 120 amino acids or more) away from the N-terminus of the extracellular domain. In some embodiments, the binding moiety binds to a region (e.g. epitope) in the extracellular domain that is within about 102 amino acids (e.g. within about any of 100, 90, 80, 70, 60, 50, 40, or 30 amino acids) from the C-terminus of the extracellular domain. In some embodiments, the target molecule is a transmembrane receptor, such as a transmembrane receptor selected from the group consisting of CD22, CD4, CD21 (CR2), CD30, ROR1, CD5, and CD20. In some embodiments, the target molecule is CD4. In some embodiments, the target molecule is CD22.

In some embodiments, there is provided an engineered immune cell (such as cytotoxic T cell, NK cell, or γδT cell) comprising on its surface a chimeric T cell receptor (cTCR) that comprises a binding moiety specifically binding to a target molecule on the surface of a target cell, a transmembrane domain, and an intracellular signaling domain, wherein the target molecule comprises an extracellular domain comprising two or more Ig-like domains, wherein the binding moiety specifically binds to a proximal portion of the extracellular domain, wherein the engineered immune cell is capable of killing a target cell that comprises on its surface the target molecule, and wherein the engineered immune cell has no or reduced capability of killing a target cell comprising on its surface both the target molecule and the cTCR. In some embodiments, the cTCR comprises: i) an extracellular domain comprising the binding moiety; ii) an optional linker (such as a GS linker); iii) an optional extracellular domain of a TCR subunit or a portion thereof, iii) a transmembrane domain derived from a TCR subunit, and iv) an intracellular signaling domain derived from a TCR subunit. In some embodiments, the TCR subunit is selected from the group consisting of TCRα, TCRβ, TCRγ, TCRδ, CD3γ, and CD3ε. In some embodiments, the transmembrane domain, the intracellular signaling domain, and the optional extracellular domain of a TCR subunit or a portion thereof are derived from the same TCR subunit. In some embodiments, the transmembrane domain, the intracellular signaling domain, and the optional extracellular domain of a TCR subunit or a portion thereof are derived from CD3ε. In some embodiments, the cTCR comprises the binding moiety fused to the N-terminus of a full length CD3ε. In some embodiments, the binding moiety binds to a region (e.g. epitope) outside the first Ig-like domain at the N-terminal end of the extracellular domain. In some embodiments, the binding moiety binds to a region (e.g. epitope) within the first two Ig-like domains from the C-terminal end of the extracellular domain. In some embodiments, the target molecule is a transmembrane receptor, such as a transmembrane receptor selected from the group consisting of CD22, CD4, CD21 (CR2), CD30, ROR1, CD5, and CD20. In some embodiments, the target molecule is CD4. In some embodiments, the target molecule is CD22.

### Immune Cells

Exemplary engineered immune cells useful for the present invention include, but are not limited to, dendritic cells (including immature dendritic cells and mature dendritic cells), T lymphocytes (such as naïve T cells, effector T cells, memory T cells, cytotoxic T lymphocytes, T helper cells, Natural Killer T cells, Treg cells, tumor infiltrating lymphocytes (TIL), and lymphokine-activated killer (LAK) cells), B cells, Natural Killer (NK) cells, NKT cells, aPT cells, γδT cells, monocytes, macrophages, neutrophils, granulocytes, peripheral blood mononuclear cells (PBMC) and combinations thereof. Subpopulations of immune cells can be defined by the presence or absence of one or more cell surface markers known in the art (e.g., CD3, CD4, CD8, CD19, CD20, CD11c, CD123, CD56, CD34, CD14, CD33, etc.). In the cases that the pharmaceutical composition comprises a plurality of engineered mammalian immune cells, the engineered mammalian immune cells can be a specific subpopulation of an immune cell type, a combination of subpopulations of an immune cell type, or a combination of two or more immune cell types. In some embodiments, the immune cell is present in a homogenous cell population. In some embodiments, the immune cell is present in a heterogeneous cell population that is enhanced in the immune cell. In some embodiments, the engineered immune cell is a lymphocyte. In some embodiments, the engineered immune cell is not a lymphocyte. In some embodiments, the engineered immune cell is suitable for adoptive immunotherapy. In some embodiments, the engineered immune cell is a PBMC. In some embodiments, the engineered immune cell is an immune cell derived from the PBMC. In some embodiments, the engineered immune cell is a T cell. In some embodiments, the engineered immune cell is a CD4⁺ T cell. In some embodiments, the engineered immune cell is a CD8⁺ T cell. In some embodiments, the therapeutic cell is a T cell expressing TCRα and TCRβ chains (i.e., αβ T cell). In some embodiments, the therapeutic cell is a T cell expressing TCRγ and TCRδ chains (i.e., γδ T cell). In some embodiments, the therapeutic cell is a γ9δ2 T cell. In some embodiments, the therapeutic cell is a δ1 T cell. In some embodiments, the therapeutic cell is a δ3 T cell. In some embodiments, the engineered immune cell is a B cell. In some embodiments, the engineered immune cell is an NK cell. In some embodiments, the engineered immune cell is an NK-T cell. In some embodiments, the engineered immune cell is a dendritic cell (DC). In some embodiments, the engineered immune cell is a DC-activated T cell.

In some embodiments, the engineered immune cell is derived from a primary cell. In some embodiments, the engineered immune cell is a primary cell isolated from an individual. In some embodiments, the engineered immune cell is propagated (such as proliferated and/or differentiated) from a primary cell isolated from an individual. In some embodiments, the primary cell is obtained from the thymus. In some embodiments, the primary cell is obtained from the lymph or lymph nodes (such as tumor draining lymph nodes). In some embodiments, the primary cell is obtained from the spleen. In some embodiments, the primary cell is obtained from the bone marrow. In some embodiments, the primary cell is obtained from the blood, such as the peripheral blood. In some embodiments, the primary cell is a Peripheral Blood Mononuclear Cell (PBMC). In some embodiments, the primary cell is derived from the blood plasma. In some embodiments, the primary cell is derived from a tumor. In some embodiments, the primary cell is obtained from the mucosal immune system. In some embodiments, the primary cell is obtained from a biopsy sample.

In some embodiments, the engineered immune cell is derived from a cell line. In some embodiments, the engineered immune cell is obtained from a commercial cell line. In some embodiments, the engineered immune cell is propagated (such as proliferated and/or differentiated) from a cell line established from a primary cell isolated from an individual. In some embodiments, the cell line is mortal. In some embodiments, the cell line is immortalized. In some embodiments, the cell line is a tumor cell line, such as a leukemia or lymphoma cell line. In some embodiments, the cell line is a cell line derived from the PBMC. In some embodiments, the cell line is a stem cell line. In some embodiments, the cell line is NK-92.

In some embodiments, the engineered immune cell is derived from a stem cell. In some embodiments, the stem cell is an embryonic stem cell (ESC). In some embodiments, the stem cell is hematopoietic stem cell (HSC). In some embodiments, the stem cell is a mesenchymal stem cell. In some embodiments, the stem cell is an induced pluripotent stem cell (iPSC).

### Co-Receptor (“COR”)

In some embodiments, the engineered immune cells further comprise one or more co-receptors (“COR”).

In some embodiments, the COR facilitates the migration of the immune cell to follicles. In some embodiments, the COR facilitates the migration of the immune cell to the gut. In some embodiments, the COR facilitates the migration of the immune cells to the skin.

In some embodiments, the COR is CXCR5. In some embodiments, the COR is CCR9. In some embodiments, the COR is α4β7 (also referred to as integrin α4β7). In some embodiments, the engineered immune cell comprises two or more receptors selected from the group consisting of CXCR5, α4β7, and CCR9. In some embodiments, the engineered immune cell comprises both α4β7 and CCR9. In some embodiments, the engineered immune cell comprises CXCR5, α4β7, and CCR9.

CCR9, also known as C-C chemokine receptor type 9 (CCR9), is a member of the beta chemokine receptor family and mediates chemotaxis in response to its binding ligand, CCL25. CCR9 is predicted to be a seven transmembrane domain protein similar in structure to a G protein-coupled receptor. CCR9 is expressed on T cells in the thymus and small intestine, and it plays a role in regulating the development and migration of T lymphocytes (Uehara, S., et al. (2002) J. Immunol. 168(6):2811-2819). CCR9/CCL25 has been shown to direct immune cells to the small intestine (Pabst, O., et al. (2004). J. Exp. Med. 199(3):411). Co-expressing a CCR9 in the immune cells can thus direct the engineered immune cells to the gut. In some embodiments, a splicing variant of CCR9 is used.

α4β7, or lymphocyte Peyer patch adhesion molecule (LPAM), is an integrin that is expressed on lymphocytes and that is responsible for T-cell homing into gut-associated lymphoid tissue (Petrovic, A. et al. (2004) Blood 103(4):1542-1547). α4β7 is a heterodimer comprised of CD49d (the protein product of ITGA4, the gene encoding the α4 integrin subunit) and ITGB7 (the protein product of ITGB4, the gene encoding the 37 integrin subunit). In some embodiments, a splicing variant of α4 is incorporated into the α4β7 heterodimer. In some embodiments, a splicing variant of β7 is incorporated into the α4β7 heterodimer. In other embodiments, splicing variants of α4 and splicing variants of β7 are incorporated into the heterodimer. Co-expression of α4β7, alone or in combination of CCR9, can direct the engineered immune cells to the gut.

Although α4β7 and CCR9 both function in homing to the gut, they are not necessarily co-regulated. The vitamin A metabolite retinoic acid plays a role in the induction of expression of both CCR9 and α4β7. α4β7 expression, however, can be induced through other means, while CCR9 expression requires retinoic acid. Furthermore, colon-tropic T-cells express only α4β7 and not CCR9, showing that the two receptors are not always coexpressed or coregulated. (See Takeuchi, H., et al. J. Immunol. (2010) 185(9):5289-5299.)

In some embodiments, CCR9 and α4β7 function as CORs for targeting the engineered immune cell to the gut.

In some embodiments, the immune cell expresses CXCR5, also known as C-X-C chemokine receptor type 5. CXCR5 is a G protein-coupled receptor containing seven transmembrane domains that belongs to the CXC chemokine receptor family CXCR5 and its ligand, the chemokine CXCL13, play a central role in trafficking lymphocytes to follicles within secondary lymphoid tissues, including lymph nodes and the spleen. (Bürkle, A. et al. (2007) Blood 110:3316-3325.) In particular, CXCR5 enables T cells to migrate to lymph node B cell zones in response to CXCL13 (Schaerli, P. et al. (2000) J. Exp. Med. 192(11):1553-1562.) When expressed in the immune cell, CXCR5 can function as a COR for targeting the engineered immune cells to follicles. In some embodiments, a splicing variant of CXCR5 is used.

In general, a non-naturally occurring variant of any of the CORs discussed above can be comprised/expressed in the engineered immune cells. These variants may, for example, contain one or more mutations, but nonetheless maintain some or more functions of the corresponding native receptors. For example, in some embodiments, the COR is a variant of a naturally occurring CCR9, α4β, or CXCR5, wherein the variant has an amino acid sequence that is at least about any of 90%, 95%, 96%, 97%, 98%, or 99% identical to a native CCR9, α4β, or CXCR5. In some embodiments, the COR is a variant of a naturally occurring CCR9, α4β, or CXCR5, wherein the variant comprises no more than about any one of 1, 2, 3, 4, 5, 6, 7, 8, 9, or 10 amino acid substitutions as compared to that of a native CCR9, α4β, or CXCR5.

In some embodiments, the COR is a chemokine receptor. In some embodiments, the COR is an integrin. In some embodiments, the COR is selected from the group consisting of CCR1, CCR2, CCR3, CCR4, CCR5, CCR6, CCR7, CCR8, CCR9, CCR10, CXCR1, CXCR2, CXCR3, CXCR4, CXCR5, CXCR6, CX3CR1, XCR1, ACKR1, ACKR2, ACKR3, ACKR4, and CCRL2.

In some embodiments, the COR is not normally expressed in the immune cell from which the engineered immune cell is derived from. In some embodiments, the COR is expressed at low levels in the immune cell from which the engineered immune cell is derived from.

### Anti-HIV Antibodies

The engineered immune cells described herein in some embodiments further express (and secrete) an anti-HIV antibody, such as a broadly neutralizing antibody. bNAbs were first discovered in elite controllers, who were infected with HIV, but could naturally control the virus infection without taking antiretroviral medicines. bNAbs are neutralizing antibodies, which neutralize multiple HIV viral strains. bNAbs target conserved epitopes of the virus, even if the virus undergoes mutations. The engineered immune cells described herein in some embodiments can secrete a broadly neutralizing antibody to block HIV infection of other host cells.

In some embodiments, the bNAb specifically recognizes a viral epitope on MPER of gp41, V1V2 glycan, outer domain of glycan, V3 glycan, or a CD4 binding site. A bNAb may block the interaction of the virus envelop glycoprotein with CD4. See, Mascola and Haynes, Immunol. Rev. 2013 July; 254(1):225-44.

Suitable bNAbs include, but are not limited to, VRC01, PGT-121, 3BNC117, 10-1074, UB-421, N6, VRC07, VRC07-523, eCD4-IG, 10E8, 10E8v4, PG9, PGDM 1400, PGT151, CAP256.25, 35022, and 8ANC195. See, Science Translational Medicine, 23 Dec. 2015: Vol. 7, Issue 319, pp. 319ra206; PLoS Pathog. 2013; 9(5):e1003342; 2015 Jun. 25; 522(7557):487-91; Nat Med. 2017 February; 23(2):185-191; and Nature Immunology, volume 19, pages 1179-1188 (2018). Other suitable broadly neutralizing antibodies can be found at, for example, Cohen et al., Current Opin. HIV AIDS, 2018 Jul.; 13(4):366-373; and Mascola and Haynes, Immunol. Rev. 2013 July; 254(1):225-44.

### Methods of Preparation

Also provided are compositions and methods for preparing the recognition molecules and engineered immune cells described herein.

### Antibody Moieties

In some embodiments, the binding moieties described herein comprise an antibody moiety (for example anti-CD22 D1-4 antibody moiety and anti-CD22 D5-7 antibody moiety). In some embodiments, the antibody moiety comprises VH and VL domains, or variants thereof, from the monoclonal antibody. In some embodiments, the antibody moiety further comprises C_{H}1 and C_{L} domains, or variants thereof, from the monoclonal antibody. Monoclonal antibodies can be prepared, e.g., using hybridoma methods, such as those described by Kohler and Milstein, Nature, 256:495 (1975) and Sergeeva et al., Blood, 117(16):4262-4272.

In a hybridoma method, a hamster, mouse, or other appropriate host animal is typically immunized with an immunizing agent to elicit lymphocytes that produce or are capable of producing antibodies that will specifically bind to the immunizing agent. Alternatively, the lymphocytes can be immunized in vitro. The immunizing agent can include a polypeptide or a fusion protein of the protein of interest, or a complex comprising at least two molecules, such as a complex comprising a peptide and an MHC protein. Generally, peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which prevents the growth of HGPRT-deficient cells.

In some embodiments, the immortalized cell lines fuse efficiently, support stable high-level expression of antibody by the selected antibody-producing cells, and are sensitive to a medium such as HAT medium. In some embodiments, the immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies. Kozbor, J. Immunol., 133:3001 (1984); Brodeur et al. Monoclonal Antibody Production Techniques and Applications (Marcel Dekker, Inc.: New York, 1987) pp. 51-63.

The culture medium in which the hybridoma cells are cultured can then be assayed for the presence of monoclonal antibodies directed against the polypeptide. The binding specificity of monoclonal antibodies produced by the hybridoma cells can be determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107: 220 (1980).

After the desired hybridoma cells are identified, the clones can be sub-cloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the sub-clones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In some embodiments, the antibody moiety comprises sequences from a clone selected from an antibody moiety library (such as a phage library presenting scFv or Fab fragments). The clone may be identified by screening combinatorial libraries for antibody fragments with the desired activity or activities. For example, a variety of methods are known in the art for generating phage display libraries and screening such libraries for antibodies possessing the desired binding characteristics. Such methods are reviewed, e.g., in Hoogenboom et al., Methods in Molecular Biology 178:1-37 (O'Brien et al., ed., Human Press, Totowa, N.J., 2001) and further described, e.g., in McCafferty et al., Nature 348:552-554; Clackson et al., Nature 352: 624-628 (1991); Marks et al., J. Mol. Biol. 222: 581-597 (1992); Marks and Bradbury, Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

The antibody moiety can be prepared using phage display to screen libraries for antibodies specific to the target antigen (such as a CD4 or CD22 polypeptides). The library can be a human scFv phage display library having a diversity of at least one x 10⁹ (such as at least about any of 1×10⁹, 2.5×10⁹, 5×10⁹, 7.5×10⁹, 1×10¹⁰, 2.5×10¹⁰, 5×10¹⁰, 7.5×10¹⁰, or 1×10¹¹) unique human antibody fragments. In some embodiments, the library is a naïve human library constructed from DNA extracted from human PMBCs and spleens from healthy donors, encompassing all human heavy and light chain subfamilies. In some embodiments, the library is a naïve human library constructed from DNA extracted from PBMCs isolated from patients with various diseases, such as patients with autoimmune diseases, cancer patients, and patients with infectious diseases. In some embodiments, the library is a semi-synthetic human library, wherein heavy chain CDR3 is completely randomized, with all amino acids (with the exception of cysteine) equally likely to be present at any given position (see, e.g., Hoet, R. M. et al., Nat. Biotechnol. 23(3):344-348, 2005). In some embodiments, the heavy chain CDR3 of the semi-synthetic human library has a length from about 5 to about 24 (such as about any of 5, 6, 7, 8, 9, 10, 11, 12, 13, 14, 15, 16, 17, 18, 19, 20, 21, 22, 23, or 24) amino acids. In some embodiments, the library is a fully synthetic phage display library. In some embodiments, the library is a non-human phage display library.

Phage clones that bind to the target antigen with high affinity can be selected by iterative binding of phage to the target antigen, which is bound to a solid support (such as, for example, beads for solution panning or mammalian cells for cell panning), followed by removal of non-bound phage and by elution of specifically bound phage. In an example of solution panning, the target antigen can be biotinylated for immobilization to a solid support. The biotinylated target antigen is mixed with the phage library and a solid support, such as streptavidin-conjugated Dynabeads M-280, and then target antigen-phage-bead complexes are isolated. The bound phage clones are then eluted and used to infect an appropriate host cell, such as E. coli XL1-Blue, for expression and purification. In an example of cell panning, cells expressing CD4 or CD22 are mixed with the phage library, after which the cells are collected and the bound clones are eluted and used to infect an appropriate host cell for expression and purification. The panning can be performed for multiple (such as about any of 2, 3, 4, 5, 6 or more) rounds with either solution panning, cell panning, or a combination of both, to enrich for phage clones binding specifically to the target antigen. Enriched phage clones can be tested for specific binding to the target antigen by any methods known in the art, including for example ELISA and FACS.

In some embodiments, the CD22 binding moieties bind to the same epitope as a reference antibody. In some embodiments, the CD22 binding moieties compete for binding with a reference antibody. Competition assays can be used to determine whether two antibodies moieties bind the same epitope (or compete with each other) by recognizing identical or sterically overlapping epitopes or one antibody competitively inhibits binding of another antibody to the antigen. Exemplary competition assays include, but are not limited to, routine assays such as those provided in Harlow and Lane (1988) Antibodies: A Laboratory Manual ch.14 (Cold Spring Harbor Laboratory, Cold Spring Harbor, N.Y.). Detailed exemplary methods for mapping an epitope to which an antibody binds are provided in Morris (1996) “Epitope Mapping Protocols,” in Methods in Molecular Biology vol. 66 (Humana Press, Totowa, N.J.). In some embodiments, two antibodies are said to bind to the same epitope if each blocks binding of the other by 50% or more.

### Human and Humanized Antibody Moieties

The antibody moieties described herein can be human or humanized. Humanized forms of non-human (e.g., murine) antibody moieties are chimeric immunoglobulins, immunoglobulin chains, or fragments thereof (such as Fv, Fab, Fab′, F(ab′)2, scFv, or other antigen-binding subsequences of antibodies) that typically contain minimal sequence derived from non-human immunoglobulin. Humanized antibody moieties include human immunoglobulins, immunoglobulin chains, or fragments thereof (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibody moieties can also comprise residues that are found neither in the recipient antibody moiety nor in the imported CDR or framework sequences. In general, the humanized antibody moiety can comprise substantially all of at least one, and typically two, variable domains, in which all or substantially all of the CDR regions correspond to those of a non-human immunoglobulin, and all or substantially all of the FR regions are those of a human immunoglobulin consensus sequence. See, e.g., Jones et al., Nature, 321: 522-525 (1986); Riechmann et al., Nature, 332: 323-329 (1988); Presta, Curr. Op. Struct. Biol., 2:593-596 (1992).

Generally, a humanized antibody moiety has one or more amino acid residues introduced into it from a source that is non-human. These non-human amino acid residues are often referred to as “import” residues, which are typically taken from an “import” variable domain. According to some embodiments, humanization can be essentially performed following the method of Winter and co-workers (Jones et al., Nature, 321: 522-525 (1986); Riechmann et al., Nature, 332: 323-327 (1988); Verhoeyen et al., Science, 239: 1534-1536 (1988)), by substituting rodent CDRs or CDR sequences for the corresponding sequences of a human antibody moiety. Accordingly, such “humanized” antibody moieties are antibody moieties (U.S. Pat. No. 4,816,567), wherein substantially less than an intact human variable domain has been substituted by the corresponding sequence from a non-human species. In practice, humanized antibody moieties are typically human antibody moieties in which some CDR residues and possibly some FR residues are substituted by residues from analogous sites in rodent antibodies.

As an alternative to humanization, human antibody moieties can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., PNAS USA, 90:2551 (1993); Jakobovits et al., Nature, 362:255-258 (1993); Bruggemann et al., Year in Immunol., 7:33 (1993); U.S. Pat. Nos. 5,545,806, 5,569,825, 5,591,669; 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); Lonberg and Huszar, Intern. Rev. Immunol., 13: 65-93 (1995).

Human antibodies may also be generated by in vitro activated B cells (see U.S. Pat. Nos. 5,567,610 and 5,229,275) or by using various techniques known in the art, including phage display libraries. Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147(1): 86-95 (1991).

### Antibody Variants

In some embodiments, amino acid sequence variants of the antigen-binding domains (e.g., anti-CD22 D1-4 antibody moiety, anti-CD22 D5-7 antibody moiety, and anti-CD4 antibody moieties) provided herein are contemplated. For example, it may be desirable to improve the binding affinity and/or other biological properties of the antigen-binding domain. Amino acid sequence variants of an antigen-binding domain may be prepared by introducing appropriate modifications into the nucleotide sequence encoding the antigen-binding domain, or by peptide synthesis. Such modifications include, for example, deletions from, and/or insertions into and/or substitutions of residues within the amino acid sequences of the antigen-binding domain. Any combination of deletion, insertion, and substitution can be made to arrive at the final construct, provided that the final construct possesses the desired characteristics, e.g., antigen-binding.

In some embodiments, antigen-binding domain variants having one or more amino acid substitutions are provided. Sites of interest for substitutional mutagenesis include the HVRs and FRs of antibody moieties. Amino acid substitutions may be introduced into an antigen-binding domain of interest and the products screened for a desired activity, e.g., retained/improved antigen binding or decreased immunogenicity.

Conservative substitutions are shown in Table 2 below. Variant CORS discussed herein can also contain such conservative substitutions.

TABLE 2
CONSERVATIVE SUBSTITITIONS

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties:
a. hydrophobic: Norleucine, Met, Ala, Val, Leu, Ile;
b. neutral hydrophilic: Cys, Ser, Thr, Asn, Gln;
c. acidic: Asp, Glu;
d. basic: His, Lys, Arg;
e. residues that influence chain orientation: Gly, Pro;
f. aromatic: Trp, Tyr, Phe.

Non-conservative substitutions will entail exchanging a member of one of these classes for another class.

An exemplary substitutional variant is an affinity matured antibody moiety, which may be conveniently generated, e.g., using phage display-based affinity maturation techniques. Briefly, one or more CDR residues are mutated and the variant antibody moieties displayed on phage and screened for a particular biological activity (e.g., binding affinity). Alterations (e.g., substitutions) may be made in HVRs, e.g., to improve antibody moiety affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or specificity determining residues (SDRs), with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).)

In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody moiety variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody moiety to bind antigen. For example, conservative alterations (e.g., conservative substitutions as provided herein) that do not substantially reduce binding affinity may be made in HVRs. Such alterations may be outside of HVR “hotspots” or SDRs. In some embodiments of the variant V_{H} and V_{L} sequences provided above, each HVR either is unaltered, or contains no more than one, two or three amino acid substitutions.

A useful method for identification of residues or regions of an antigen-binding domain that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antigen-binding domain with antigen is affected. Further substitutions may be introduced at the amino acid locations demonstrating functional sensitivity to the initial substitutions. Alternatively, or additionally, a crystal structure of an antigen-antigen-binding domain complex can be determined to identify contact points between the antigen-binding domain and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antigen-binding domain with an N-terminal methionyl residue. Other insertional variants of the antigen-binding domain include the fusion to the N- or C-terminus of the antigen-binding domain to an enzyme (e.g., for ADEPT) or a polypeptide which increases the serum half-life of the antigen-binding domain.

### Nucleic Acids

Also provided herein are nucleic acids (or a set of nucleic acids) encoding the recognition molecules (or one or more portions thereof), COR, and/or bNAb described herein, as well as vectors comprising the nucleic acid(s).

The expression of the recognition molecules (or one or more portions thereof), COR, and/or bNAb can be achieved by inserting the nucleic acid(s) into an appropriate expression vector, such that the nucleic acid(s) is operably linked to 5′ and/or 3′ regulatory elements, including for example a promoter (e.g., a lymphocyte-specific promoter) and a 3′ untranslated region (UTR). The vectors can be suitable for replication and integration in host cells. Typical cloning and expression vectors contain transcription and translation terminators, initiation sequences, and promoters useful for regulation of the expression of the desired nucleic acid sequence.

The nucleic acid(s) can be cloned into a number of types of vectors. For example, the nucleic acid can be cloned into a vector including, but not limited to, a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art. Viruses that are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatinine kinase promoter.

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, β-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Exemplary methods to confirm the presence of the nucleic acid(s) in the mammalian cell, include, for example, molecular biological assays well known to those of skill in the art, such as Southern and Northern blotting, RT-PCR and PCR; biochemical assays, such as detecting the presence or absence of a particular peptide, e.g., by immunological methods (such as ELISAs and Western blots).

In some embodiments, the one or more nucleic acid sequences are contained in separate vectors. In some embodiments, at least some of the nucleic acid sequences are contained in the same vector. In some embodiments, all of the nucleic acid sequences are contained in the same vector. Vectors may be selected, for example, from the group consisting of mammalian expression vectors and viral vectors (such as those derived from retroviruses, adenoviruses, adeno-associated viruses, herpes viruses, and lentiviruses).

For example, in some embodiments, the nucleic acid comprises a first nucleic acid sequence encoding the immune cell receptor polypeptide chain, optionally a second nucleic acid encoding the COR polypeptide chain, and optionally a third nucleic acid encoding a bNAb polypeptide. In some embodiments, the first nucleic acid sequence is contained in a first vector, the optional second nucleic acid sequence is contained in a second vector, and the optional third nucleic acid sequence is contained in a third vector. In some embodiments, the first and second nucleic acid sequences are contained in a first vector, and the third nucleic acid sequence is contained in a second vector. In some embodiments, the first and third nucleic acid sequences are contained in a first vector, and the second nucleic acid sequence is contained in a second vector. In some embodiments, the second and third nucleic acid sequences are contained in a first vector, and the first nucleic acid sequence is contained in a second vector. In some embodiments, the first, second, and third nucleic acid sequences are contained in the same vector. In some embodiments, the first, second, and third nucleic acids can be connected to each other via a linker selected from the group consisting of an internal ribosomal entry site (IRES) and a nucleic acid encoding a self-cleaving 2 Å peptide (such as P2A, T2A, E2A, or F2A).

In some embodiments, the first nucleic acid sequence is under the control of a first promoter, the optional second nucleic acid sequence is under the control of a second promoter, and the optional third nucleic acid sequence is under the control of a third promoter. In some embodiments, some or all of the first, second, and third promoters have the same sequence. In some embodiments, some or all of the first, second, and third promoters have different sequences. In some embodiments, some or all of the first, second, and third, nucleic acid sequences are expressed as a single transcript under the control of a single promoter in a multicistronic vector. In some embodiments, one or more of the promoters are inducible.

In some embodiments, some or all of the first, second, and third nucleic acid sequences have similar (such as substantially or about the same) expression levels in an immune cell (such as a T cell). In some embodiments, some of the first, second, and third nucleic acid sequences have expression levels in an immune cell (such as a T cell) that differ by at least about two (such as at least about any of 2, 3, 4, 5, or more) times. Expression can be determined at the mRNA or protein level. The level of mRNA expression can be determined by measuring the amount of mRNA transcribed from the nucleic acid using various well-known methods, including Northern blotting, quantitative RT-PCR, microarray analysis and the like. The level of protein expression can be measured by known methods including immunocytochemical staining, enzyme-linked immunosorbent assay (ELISA), western blot analysis, luminescent assays, mass spectrometry, high performance liquid chromatography, high-pressure liquid chromatography-tandem mass spectrometry, and the like.

Methods of introducing and expressing genes into a cell (such as immune cell) are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human, cells. Other viral vectors can be derived from lentivirus, poxviruses, herpes simplex virus 1, adenoviruses and adeno-associated viruses, and the like.

Chemical means for introducing a polynucleotide into a host cell (such as immune cell) include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances that may be naturally occurring or synthetic lipids. For example, lipids include the fatty droplets that naturally occur in the cytoplasm as well as the class of compounds that contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

The nucleic acids described herein may be transiently or stably incorporated in the immune cells. In some embodiments, the nucleic acid is transiently expressed in the engineered immune cell. For example, the nucleic acid may be present in the nucleus of the engineered immune cell in an extrachromosomal array comprising the heterologous gene expression cassette. Nucleic acids may be introduced into the engineered mammalian using any transfection or transduction methods known in the art, including viral or non-viral methods. Exemplary non-viral transfection methods include, but are not limited to, chemical-based transfection, such as using calcium phosphate, dendrimers, liposomes, or cationic polymers (e.g., DEAE-dextran or polyethylenimine); non-chemical methods, such as electroporation, cell squeezing, sonoporation, optical transfection, impalefection, protoplast fusion, hydrodynamic delivery, or transposons; particle-based methods, such as using a gene gun, magnectofection or magnet assisted transfection, particle bombardment; and hybrid methods, such as nucleofection. In some embodiments, the nucleic acid is a DNA. In some embodiments, the nucleic acid is a RNA. In some embodiments, the nucleic acid is linear. In some embodiments, the nucleic acid is circular.

In some embodiments, the nucleic acid(s) is present in the genome of the engineered immune cell. For example, the nucleic acid(s) may be integrated into the genome of the immune cell by any methods known in the art, including, but not limited to, virus-mediated integration, random integration, homologous recombination methods, and site-directed integration methods, such as using site-specific recombinase or integrase, transposase, Transcription activator-like effector nuclease (TALEN©), CRISPR/Cas9, and zinc-finger nucleases. In some embodiments, the nucleic acid(s) is integrated in a specifically designed locus of the genome of the engineered immune cell. In some embodiments, the nucleic acid(s) is integrated in an integration hotspot of the genome of the engineered immune cell. In some embodiments, the nucleic acid(s) is integrated in a random locus of the genome of the engineered immune cell. In the cases that multiple copies of the nucleic acids are present in a single engineered immune cell, the nucleic acid(s) may be integrated in a plurality of loci of the genome of the engineered immune cell.

The nucleic acid(s) encoding the recognition molecules, COR, and/or bNAb can be operably linked to a promoter. In some embodiments, the promoter is an endogenous promoter. For example, the nucleic acid(s) encoding the recognition molecule, COR, or bNAb may be knocked-in to the genome of the engineered immune cell downstream of an endogenous promoter using any methods known in the art, such as CRISPR/Cas9 method. In some embodiments, the endogenous promoter is a promoter for an abundant protein, such as beta-actin. In some embodiments, the endogenous promoter is an inducible promoter, for example, inducible by an endogenous activation signal of the engineered immune cell. In some embodiments, wherein the engineered immune cell is a T cell, the promoter is a T cell activation-dependent promoter (such as an IL-2 promoter, an NFAT promoter, or an NFκB promoter).

In some embodiments, the promoter is a heterologous promoter.

In some embodiments, the nucleic acid(s) encoding the recognition molecule, COR, and/or bNAb is operably linked to a constitutive promoter. In some embodiments, the nucleic acid(s) encoding the recognition molecule, COR or bNAb is operably linked to an inducible promoter. In some embodiments, a constitutive promoter is operably linked to the nucleic acid(s) encoding a recognition molecule, and an inducible promoter is operably linked to a nucleic acid encoding a COR or bNAb. In some embodiments, a first inducible promoter is operably linked to a nucleic acid encoding a recognition molecule, and an second inducible promoter is operably linked to a nucleic acid encoding a COR, or vice versa. In some embodiments, a first inducible promoter is operably linked to a nucleic acid encoding a recognition molecule, and a second inducible promoter is operably linked to a nucleic acid encoding bNAb, or vice versa. In some embodiments, a first inducible promoter is operably linked to a nucleic acid encoding a COR, and a second inducible promoter is operably linked to a nucleic acid encoding bNAb or vice versa. In some embodiments, the first inducible promoter is inducible by a first inducing condition, and the second inducible promoter is inducible by a second inducing condition. In some embodiments, the first inducing condition is the same as the second inducing condition. In some embodiments, the first inducible promoter and the second inducible promoter are induced simultaneously. In some embodiments, the first inducible promoter and the second inducible promoter are induced sequentially, for example, the first inducible promoter is induced prior to the second inducible promoter, or the first inducible promoter is induced after the second inducible promoter.

Constitutive promoters allow heterologous genes (also referred to as transgenes) to be expressed constitutively in the host cells. Exemplary constitutive promoters contemplated herein include, but are not limited to, Cytomegalovirus (CMV) promoters, human elongation factors-1alpha (hEF1α), ubiquitin C promoter (UbiC), phosphoglycerokinase promoter (PGK), simian virus 40 early promoter (SV40), and chicken β-Actin promoter coupled with CMV early enhancer (CAGG). The efficiencies of such constitutive promoters on driving transgene expression have been widely compared in a huge number of studies. For example, Michael C. Milone et al compared the efficiencies of CMV, hEF1α, UbiC and PGK to drive chimeric antigen receptor expression in primary human T cells, and concluded that hEF1α promoter not only induced the highest level of transgene expression, but was also optimally maintained in the CD4 and CD8 human T cells (Molecular Therapy, 17(8): 1453-1464 (2009)). In some embodiments, the promoter in the nucleic acid is a hEF1α promoter.

The inducible promoter can be induced by one or more conditions, such as a physical condition, microenvironment of the engineered immune cell, or the physiological state of the engineered immune cell, an inducer (i.e., an inducing agent), or a combination thereof. In some embodiments, the inducing condition does not induce the expression of endogenous genes in the engineered immune cell, and/or in the subject that receives the pharmaceutical composition. In some embodiments, the inducing condition is selected from the group consisting of: inducer, irradiation (such as ionizing radiation, light), temperature (such as heat), redox state, tumor environment, and the activation state of the engineered immune cell.

In some embodiments, the promoter is inducible by an inducer. In some embodiments, the inducer is a small molecule, such as a chemical compound. In some embodiments, the small molecule is selected from the group consisting of doxycycline, tetracycline, alcohol, metal, or steroids. Chemically-induced promoters have been most widely explored. Such promoters includes promoters whose transcriptional activity is regulated by the presence or absence of a small molecule chemical, such as doxycycline, tetracycline, alcohol, steroids, metal and other compounds. Doxycycline-inducible system with reverse tetracycline-controlled transactivator (rtTA) and tetracycline-responsive element promoter (TRE) is the most mature system at present. WO9429442 describes the tight control of gene expression in eukaryotic cells by tetracycline responsive promoters. WO9601313 discloses tetracycline-regulated transcriptional modulators. Additionally, Tet technology, such as the Tet-on system, has described, for example, on the website of TetSystems.com. Any of the known chemically regulated promoters may be used to drive expression of the therapeutic protein in the present application.

In some embodiments, the inducer is a polypeptide, such as a growth factor, a hormone, or a ligand to a cell surface receptor, for example, a polypeptide that specifically binds a tumor antigen. In some embodiments, the polypeptide is expressed by the engineered immune cell. In some embodiments, the polypeptide is encoded by a nucleic acid in the nucleic acid. Many polypeptide inducers are also known in the art, and they may be suitable for use in the present invention. For example, ecdysone receptor-based gene switches, progesterone receptor-based gene switches, and estrogen receptor based gene switches belong to gene switches employing steroid receptor derived transactivators (WO9637609 and WO9738117 etc.).

In some embodiments, the inducer comprises both a small molecule component and one or more polypeptides. For example, inducible promoters that dependent on dimerization of polypeptides are known in the art, and may be suitable for use in the present invention. The first small molecule CID system, developed in 1993, used FK1012, a derivative of the drug FK506, to induce homo-dimerization of FKBP. By employing similar strategies, Wu et al successfully make the CAR-T cells titratable through an ON-switch manner by using Rapalog/FKPB-FRB* and Gibberelline/GID1-GAI dimerization dependent gene switch (C.-Y. Wu et al., Science 350, aab4077 (2015)). Other dimerization dependent switch systems include Coumermycin/GyrB-GyrB (Nature 383 (6596): 178-81), and HaXS/Snap-tag-HaloTag (Chemistry and Biology 20 (4): 549-57).

In some embodiments, the promoter is a light-inducible promoter, and the inducing condition is light. Light inducible promoters for regulating gene expression in mammalian cells are also well known in the art (see, for example, Science 332, 1565-1568 (2011); Nat. Methods 9, 266-269 (2012); Nature 500: 472-476 (2013); Nature Neuroscience 18:1202-1212 (2015)). Such gene regulation systems can be roughly put into two categories based on their regulations of (1) DNA binding or (2) recruitment of a transcriptional activation domain to a DNA bound protein. For instance, synthetic mammalian blue light controlled transcription system based on melanopsin, which, in response to blue light (480 nm), triggers an intracellular calcium increase that result in calcineurin-mediated mobilization of NFAT, were developed and tested in mammalian cells. More recently, Motta-Mena et al described a new inducible gene expression system developed from naturally occurring EL222 transcription factor that confers high-level, blue light-sensitive control of transcriptional initiation in human cell lines and zebrafish embryos (Nat. Chem. Biol. 10(3):196-202 (2014)). Additionally, the red light induced interaction of photoreceptor phytochrome B (PhyB) and phytochrome-interacting factor 6 (PIF6) of Arabidopsis thaliana was exploited for a red light triggered gene expression regulation. Furthermore, ultraviolet B (UVB)-inducible gene expression system were also developed and proven to be efficient in target gene transcription in mammalian cells (Chapter 25 of Gene and Cell Therapy: Therapeutic Mechanisms and Strategies, Fourth Edition CRC Press, Jan. 20, 2015). Any of the light-inducible promoters described herein may be used to drive expression of the therapeutic protein in the present invention.

In some embodiments, the promoter is a light-inducible promoter that is induced by a combination of a light-inducible molecule, and light. For example, a light-cleavable photocaged group on a chemical inducer keeps the inducer inactive, unless the photocaged group is removed through irradiation or by other means. Such light-inducible molecules include small molecule compounds, oligonucleotides, and proteins. For example, caged ecdysone, caged IPTG for use with the lac operon, caged toyocamycin for ribozyme-mediated gene expression, caged doxycycline for use with the Tet-on system, and caged Rapalog for light mediated FKBP/FRB dimerization have been developed (see, for example, Curr Opin Chem Biol. 16(3-4): 292-299 (2012)).

In some embodiments, the promoter is a radiation-inducible promoter, and the inducing condition is radiation, such as ionizing radiation. Radiation inducible promoters are also known in the art to control transgene expression. Alteration of gene expression occurs upon irradiation of cells. For example, a group of genes known as “immediate early genes” can react promptly upon ionizing radiation. Exemplary immediate early genes include, but are not limited to, Erg-1, p21/WAF-1, GADD45alpha, t-PA, c-Fos, c-Jun, NF-kappaB, and AP1. The immediate early genes comprise radiation responsive sequences in their promoter regions. Consensus sequences CC(A/T)₆GG (SEQ ID NO: 65) have been found in the Erg-1 promoter, and are referred to as serum response elements or known as CArG elements. Combinations of radiation induced promoters and transgenes have been intensively studied and proven to be efficient with therapeutic benefits. See, for example, Cancer Biol Ther. 6(7):1005-12 (2007) and Chapter 25 of Gene and Cell Therapy: Therapeutic Mechanisms and Strategies, Fourth Edition CRC Press, Jan. 20, 2015. Any of the immediate early gene promoters or any promoter comprising a serum response element or SEQ ID NO: 65 may be useful as a radiation inducible promoter to drive the expression of the therapeutic protein of the present invention.

In some embodiments, the promoter is a heat inducible promoter, and the inducing condition is heat. Heat inducible promoters driving transgene expression have also been widely studied in the art. Heat shock or stress protein (HSP) including Hsp90, Hsp70, Hsp60, Hsp40, Hsp10 etc. plays important roles in protecting cells under heat or other physical and chemical stresses. Several heat inducible promoters including heat-shock protein (HSP) promoters and growth arrest and DNA damage (GADD) 153 promoters have been attempted in pre-clinical studies. The promoter of human hsp70B gene, which was first described in 1985 appears to be one of the most highly-efficient heat inducible promoters. Huang et al reported that after introduction of hsp70B-EGFP, hsp70B-TNFalpha and hsp70B-IL12 coding sequences, tumor cells expressed extremely high transgene expression upon heat treatment, while in the absence of heat treatment, the expression of transgenes were not detected. In addition, tumor growth was delayed significantly in the IL12 transgene plus heat treated group of mice in vivo (Cancer Res. 60:3435 (2000)). Another group of scientists linked the HSV-tk suicide gene to hsp70B promoter and test the system in nude mice bearing mouse breast cancer. Mice whose tumor had been administered the hsp70B-HSVtk coding sequence and heat treated showed tumor regression and a significant survival rate as compared to no heat treatment controls (Hum. Gene Ther. 11:2453 (2000)). Additional heat inducible promoters known in the art can be found in, for example, Chapter 25 of Gene and Cell Therapy: Therapeutic Mechanisms and Strategies, Fourth Edition CRC Press, Jan. 20, 2015. Any of the heat-inducible promoters discussed herein may be used to drive the expression of the therapeutic protein of the present invention.

In some embodiments, the promoter is inducible by a redox state. Exemplary promoters that are inducible by redox state include inducible promoter and hypoxia inducible promoters. For instance, Post D E et al developed hypoxia-inducible factor (HIF) responsive promoter, which specifically and strongly induce transgene expression in HIF-active tumor cells (Gene Ther. 8: 1801-1807 (2001); Cancer Res. 67: 6872-6881 (2007)).

In some embodiments, the promoter is inducible by the physiological state, such as an endogenous activation signal, of the engineered immune cell. In some embodiments, wherein the engineered immune cell is a T cell, the promoter is a T cell activation-dependent promoter, which is inducible by the endogenous activation signal of the engineered T cell. In some embodiments, the engineered T cell is activated by an inducer, such as PMA, ionomycin, or phytohaemagglutinin. In some embodiments, the engineered T cell is activated by recognition of a tumor antigen on the tumor cells via an endogenous T cell receptor, or an engineered receptor (such as recombinant TCR, or CAR). In some embodiments, the engineered T cell is activated by blockade of an immune checkpoint, such as by an immunomodulator expressed by the engineered T cell or by a second engineered immune cell. In some embodiments, the T cell activation-dependent promoter is an IL-2 promoter. In some embodiments, the T cell activation-dependent promoter is an NFAT promoter. In some embodiments, the T cell activation-dependent promoter is a NFκB promoter.

Without being bound by any theory or hypothesis, IL-2 expression initiated by the gene transcription from IL-2 promoter is a major activity of T cell activation. Un-specific stimulation of human T cells by Phorbol 12-myristate 13-acetate (PMA), or ionomycin, or phytohaemagglutinin results in IL-2 secretion from stimulated T cells. IL-2 promoter was explored for activation-induced transgene expression in genetically engineered T-cells (Virology Journal 3:97 (2006)). We found that IL-2 promoter is efficient to initiate reporter gene expression in the presence of PMA/PHA-P activation in human T cell lines. T cell receptor stimulation initiates a cascade of intracellular reactions causing an increasing of cytosolic calcium concentrations and resulting in nuclear translation of both NFAT and NFxB. Members of Nuclear Factor of Activated T cells (NFAT) are Ca²′ dependent transcription factors mediating immune response in T lymphocytes. NFAT have been shown to be crucial for inducible interleukine-2 (IL-2) expression in activated T cells (Mol Cell Biol. 15(11):6299-310 (1995); Nature Reviews Immunology 5:472-484 (2005)). We found that NFAT promoter is efficient to initiate reporter gene expression in the presence of PMA/PHA-P activation in human T cell lines. Other pathways including nuclear factor kappa B (NFκB) can also be employed to control transgene expression via T cell activation.

### Preparation of Engineered Immune Cells

The engineered immune cells may be obtained from peripheral blood, cord blood, bone marrow, tumor infiltrating lymphocytes, lymph node tissue, or thymus tissue. The host cells may include placental cells, embryonic stem cells, induced pluripotent stem cells, or hematopoietic stem cells. The cells may be obtained from humans, monkeys, chimpanzees, dogs, cats, mice, rats, and transgenic species thereof. The cells may be obtained from established cell lines.

The engineered immune cells expressing the recognition molecule, COR, and/or bNAb can be generated by introducing one or more nucleic acids (including for example a lentiviral vector) encoding the recognition molecule, COR, and/or bNAb into the immune cell. In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The nucleic acid can be inserted into a vector and packaged in retroviral particles using techniques known in the art. The recombinant virus can then be isolated and delivered to the engineered immune cell in vitro or ex vivo. A number of retroviral systems are known in the art. In some embodiments, adenovirus vectors are used. A number of adenovirus vectors are known in the art. In some embodiments, lentivirus vectors are used. In some embodiments, self-inactivating lentiviral vectors are used. For example, self-inactivating lentiviral vectors carrying the nucleic acid sequence(s) encoding the recognition molecule, COR, and/or bNAb can be packaged with protocols known in the art. The resulting lentiviral vectors can be used to transduce a mammalian cell (such as primary human T cells) using methods known in the art.

In some embodiments, the transduced or transfected mammalian cell is propagated ex vivo after introduction of the nucleic acid. In some embodiments, the transduced or transfected mammalian cell is cultured to propagate for at least about any of 1 day, 2 days, 3 days, 4 days, 5 days, 6 days, 7 days, 10 days, 12 days, or 14 days. In some embodiments, the transduced or transfected mammalian cell is cultured for no more than about any of 1 day, 2 days, 3 days, 4 days, 5 days, 6 days, 7 days, 10 days, 12 days, or 14 days. In some embodiments, the transduced or transfected mammalian cell is further evaluated or screened to select the engineered immune cell.

The introduction of the one or more nucleic acids into the immune cell can be accomplished using techniques known in the art. In some embodiments, the engineered immune cells (such as engineered T cells) are able to replicate in vivo, resulting in long-term persistence that can lead to sustained control of a disease associated with expression of the target antigen (such as viral infection).

Prior to expansion and genetic modification of the immune cells, a source of immune cells is obtained from a subject. Immune cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments of the present invention, any number of immune cell lines available in the art may be used. In some embodiments of the present invention, immune cells can be obtained from a unit of blood collected from a subject using any number of techniques known to the skilled artisan, such as FICOLL™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as Ca²⁺-free, Mg²⁺-free PBS, PlasmaLyte A, or other saline solutions with or without buffer. Alternatively, the undesirable components of the apheresis sample may be removed and the cells directly resuspended in culture media.

In some embodiments, immune cells (such as T cells) are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺ T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In some embodiments, the time period ranges from 30 minutes to 36 hours or longer (including all ranges between these values). In some embodiments, the time period is at least one, 2, 3, 4, 5, or 6 hours. In some embodiments, the time period is 10 to 24 hours. In some embodiments, the incubation time period is 24 hours. Longer incubation times may be used to isolate T cells in any situation where there are few T cells as compared to other cell types. Further, use of longer incubation times can increase the efficiency of capture of CD8⁺ T cells. Thus, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CD11b, CD 16, HLA-DR, and CD8. In some embodiments, it may be desirable to enrich for or positively select for regulatory T cells, which typically express CD4⁺, CD25⁺, CD62Lhi, GITR⁺, and FoxP3⁺. Alternatively, in some embodiments, T regulatory cells are depleted by anti-CD25 conjugated beads or other similar methods of selection.

For isolation of a desired population of cells by positive or negative selection, the concentration of cells and surface (e.g., particles such as beads) can be varied. In some embodiments, it may be desirable to significantly decrease the volume in which beads and cells are mixed together (i.e., increase the concentration of cells), to ensure maximum contact of cells and beads. For example, in some embodiments, a concentration of about 2 billion cells/ml is used. In some embodiments, a concentration of about 1 billion cells/ml is used. In some embodiments, greater than about 100 million cells/ml is used. In some embodiments, a concentration of cells of about any of 10, 15, 20, 25, 30, 35, 40, 45, or 50 million cells/ml is used. In some embodiments, a concentration of cells of about any of 75, 80, 85, 90, 95, or 100 million cells/ml is used. In some embodiments, a concentration of about 125 or about 150 million cells/ml is used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

Whether prior to or after genetic modification of the immune cells to express a desirable recognition molecules, optionally COR and optionally bNAb, the immune cells can be activated and expanded.

In some embodiments, the immune cells (such as T cells) described herein are expanded by contacting with a surface having attached thereto an agent that stimulates a CD3/TCR complex associated signal and a ligand that stimulates a co-stimulatory molecule on the surface of the T cells. In particular, T cell populations may be stimulated, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

### Genetic Modifications

In some embodiments, the engineered immune cell is a T cell modified to block or decrease the expression of CCR5. Modifications of cells to disrupt gene expression include any such techniques known in the art, including for example RNA interference (e.g., siRNA, shRNA, miRNA), gene editing (e.g., CRISPR- or TALEN-based gene knockout), and the like.

In some embodiments, engineered T cells with reduced expression of CCR5 are generated using the CRISPR/Cas system. For a review of the CRISPR/Cas system of gene editing, see for example Jian W & Marraffini L A, Annu. Rev. Microbiol. 69, 2015; Hsu P D et al., Cell, 157(6):1262-1278, 2014; and O'Connell M R et al., Nature 516: 263-266, 2014. In some embodiments, Engineered T cells with reduced expression of one or both of the endogenous TCR chains of the T cell are generated, for example using TALEN-based genome editing. In some embodiments, the engineered immune cells, in particular allogeneic immune cells obtained from donors can be modified to inactivate components of TCR involved in MHC recognition. In some embodiments, the modified immune cells do not cause graft versus host disease.

In some embodiments, the CCR5 gene (or TCR gene) is inactivated using CRISPR/Cas9 gene editing. CRISPR/Cas9 involves two main features: a short guide RNA (gRNA) and a CRISPR-associated endonuclease or Cas protein. The Cas protein is able to bind to the gRNA, which contains an engineered spacer that allows for directed targeting to, and subsequent knockout of, a gene of interest. Once targeted, the Cas protein cleaves the DNA target sequence, resulting in the knockout of the gene.

In some embodiments, the CCR5 gene (or TCR gene) is inactivated using transcription activator-like effector nuclease (TALEN©)-based genome editing. TALEN©-based genome editing involves the use of restriction enzymes that can be engineered for targeting to particular regions of DNA. A transcription activator-like effector (TALE) DNA-binding domain is fused to a DNA cleavage domain. The TALE is responsible for targeting the nuclease to the sequence of interest, and the cleavage domain (nuclease) is responsible for cleaving the DNA, resulting in the removal of that segment of DNA and subsequent knockout of the gene.

In some embodiments, the CCR5 gene (or TCR gene) is inactivated using zinc finger nuclease (ZFN) genome editing methods. Zinc finger nucleases are artificial restriction enzymes that are comprised of a zinc finger DNA-binding domain and a DNA-cleavage domain. ZFN DNA-binding domains can be engineered for targeting to particular regions of DNA. The DNA-cleavage domain is responsible for cleaving the DNA sequence of interest, resulting in the removal of that segment of DNA and subsequent knockout of the gene.

In some embodiments, the expression of the CCR5 gene is reduced by using RNA interference (RNAi) such as small interference RNA (siRNA), microRNA, and short hairpin RNA (shRNA). siRNA molecules are 20-25 nucleotide long oligonucleotide duplexes that are complementary to messenger RNA (mRNA) transcripts from genes of interest. siRNAs target these mRNAs for destruction. Through targeting, siRNAs prevent mRNA transcripts from being translated, thereby preventing the protein from being produced by the cell.

In some embodiments, the expression of the CCR5 gene (or TCR gene) is reduced by using anti-sense oligonucleotides. Antisense oligonucleotides targeting mRNA are generally known in the art and used routinely for downregulating gene expressions. See Watts, J. and Corey, D (2012) J. Pathol. 226(2):365-379.)

### Enrichment of the Engineered Immune Cells

In some embodiments, there is provided a method of enriching a heterogeneous cell population for an engineered immune cell according to any of the engineered immune cells described herein.

A specific subpopulation of engineered immune cells (such as engineered T cells) that specifically bind to a target antigen and target ligand (e.g., CD22 D1-4 or CD22 D5-7) can be enriched for by positive selection techniques. For example, in some embodiments, engineered immune cells (such as engineered T cells) are enriched for by incubation with target antigen-conjugated beads and/or target ligand-conjugated beads for a time period sufficient for positive selection of the desired engineered immune cells. In some embodiments, the time period is about 30 minutes. In some embodiments, the time period ranges from 30 minutes to 36 hours or longer (including all ranges between these values). In some embodiments, the time period is at least one, 2, 3, 4, 5, or 6 hours. In some embodiments, the time period is 10 to 24 hours. In some embodiments, the incubation time period is 24 hours. For isolation of engineered immune cells present at low levels in the heterogeneous cell population, use of longer incubation times, such as 24 hours, can increase cell yield. Longer incubation times may be used to isolate engineered immune cells in any situation where there are few engineered immune cells as compared to other cell types. The skilled artisan would recognize that multiple rounds of selection can also be used in the context of this invention.

For isolation of a desired population of engineered immune cells by positive selection, the concentration of cells and surface (e.g., particles such as beads) can be varied. In some embodiments, it may be desirable to significantly decrease the volume in which beads and cells are mixed together (i.e., increase the concentration of cells), to ensure maximum contact of cells and beads. For example, in some embodiments, a concentration of about 2 billion cells/ml is used. In some embodiments, a concentration of about 1 billion cells/ml is used. In some embodiments, greater than about 100 million cells/ml is used. In some embodiments, a concentration of cells of about any of 10, 15, 20, 25, 30, 35, 40, 45, or 50 million cells/ml is used. In some embodiments, a concentration of cells of about any of 75, 80, 85, 90, 95, or 100 million cells/ml is used. In some embodiments, a concentration of about 125 or about 150 million cells/ml is used. Using high concentrations can result in increased cell yield, cell activation, and cell expansion. Further, use of high cell concentrations allows more efficient capture of engineered immune cells that may weakly express the recognition molecule, COR, and/or bNAb.

In some embodiments, enrichment results in minimal or substantially no exhaustion of the engineered immune cells. For example, in some embodiments, enrichment results in fewer than about 50% (such as fewer than about any of 45, 40, 35, 30, 25, 20, 15, 10, or 5%) of the engineered immune cells becoming exhausted. Immune cell exhaustion can be determined by any means known in the art, including any means described herein.

In some embodiments, enrichment results in minimal or substantially no terminal differentiation of the engineered immune cells. For example, in some embodiments, enrichment results in fewer than about 50% (such as fewer than about any of 45, 40, 35, 30, 25, 20, 15, 10, or 5%) of the engineered immune cells becoming terminally differentiated. Immune cell differentiation can be determined by any methods known in the art, including any methods described herein.

In some embodiments, enrichment results in minimal or substantially no internalization of the recognition molecule or COR on the engineered immune cells. For example, in some embodiments, enrichment results in less than about 50% (such as less than about any of 45, 40, 35, 30, 25, 20, 15, 10, or 5%) of the recognition molecule or COR on the engineered immune cells becoming internalized. Internalization of the recognition molecule or COR on engineered immune cells can be determined by any methods known in the art, including any methods described herein.

In some embodiments, enrichment results in increased proliferation of the engineered immune cells. For example, in some embodiments, enrichment results in an increase of at least about 10% (such as at least about any of 20, 30, 40, 50, 60, 70, 80, 90, 100, 200, 300, 400, 500, 1000% or more) in the number of engineered immune cells following enrichment.

Thus, in some embodiments, there is provided a method of enriching a heterogeneous cell population for engineered immune cells expressing a recognition molecule (or one or more portions thereof) comprising: a) contacting the heterogeneous cell population with a first molecule comprising a target molecule (such as CD22) or one or more epitopes contained therein and/or a second molecule comprising the target molecule (such as CD22) or one or more epitopes contained therein to form complexes comprising the engineered immune cell bound to the first molecule and/or complexes comprising the engineered immune cell bound to the second molecule; and b) separating the complexes from the heterogeneous cell population, thereby generating a cell population enriched for the engineered immune cells. In some embodiments, the first and/or second molecules are immobilized, individually, to a solid support. In some embodiments, the solid support is particulate (such as beads). In some embodiments, the solid support is a surface (such as the bottom of a well). In some embodiments, the first and/or second molecules are labelled, individually, with a tag. In some embodiments, the tag is a fluorescent molecule, an affinity tag, or a magnetic tag. In some embodiments, the method further comprises eluting the engineered immune cells from the first and/or second molecules and recovering the eluate.

In some embodiments, the immune cells or engineered immune cells are enriched for CD4+ and/or CD8+ cells, for example through the use of negative enrichment, whereby cell mixtures are purified using two-step purification methods involving both physical (column) and magnetic (MACS magnetic beads) purification steps (Gunzer, M. et al. (2001) J. Immunol. Methods 258(1-2):55-63). In other embodiments, populations of cells can be enriched for CD4+ and/or CD8+ cells through the use of T cell enrichment columns specifically designed for the enrichment of CD4+ or CD8+ cells. In yet other embodiments, cell populations can be enriched for CD4+ cells through the use of commercially available kits. In some embodiments, the commercially available kit is the EASYSEP™ Human CD4+ T Cell Enrichment Kit (Stemcell Technologies). In other embodiments, the commercially available kit is the MAGNISORT™ Mouse CD4+ T cell Enrichment Kit (Thermo Fisher Scientific).

### Pharmaceutical Compositions

Also provided herein are engineered immune cell compositions (such as pharmaceutical compositions, also referred to herein as formulations) comprising an engineered immune cell (such as a T cell) described herein.

In some embodiments, there is provided an engineered immune cell composition comprising a homogeneous cell population of engineered immune cells (such as engineered T cells) of the same cell type and expressing the same recognition molecule (or one or more portions thereof), and optionally COR, and/or optionally bNAb. In some embodiments, the engineered immune cell is a T cell. In some embodiments, the engineered immune cell is selected from the group consisting of a cytotoxic T cell, a helper T cell, a natural killer T cell, and a 76T cell. In some embodiments, the engineered immune cell composition is a pharmaceutical composition.

In some embodiments, there is provided an engineered immune cell composition comprising a heterogeneous cell population comprising a plurality of engineered immune cell populations comprising engineered immune cells of different cell types, expressing different recognition molecules (or one or more portions thereof), optionally different CORs, and/or optionally different bNAbs.

In some embodiments, the pharmaceutical composition is suitable for administration to an individual, such as a human individual. In some embodiments, the pharmaceutical composition is suitable for injection. In some embodiments, the pharmaceutical composition is suitable for infusion. In some embodiments, the pharmaceutical composition is substantially free of cell culture medium. In some embodiments, the pharmaceutical composition is substantially free of endotoxins or allergenic proteins. In some embodiments, “substantially free” is less than about any of 10%, 5%, 1%, 0.1%, 0.01%, 0.001%, 1 ppm or less of total volume or weight of the pharmaceutical composition. In some embodiments, the pharmaceutical composition is free of mycoplasma, microbial agents, and/or communicable disease agents.

The pharmaceutical composition of the present applicant may comprise any number of the engineered immune cells. In some embodiments, the pharmaceutical composition comprises a single copy of the engineered immune cell. In some embodiments, the pharmaceutical composition comprises at least about any of 1, 10, 100, 1000, 10⁴, 10⁵, 10⁶, 10⁷, 10⁸ or more copies of the engineered immune cells. In some embodiments, the pharmaceutical composition comprises a single type of engineered immune cell. In some embodiments, the pharmaceutical composition comprises at least two types of engineered immune cells, wherein the different types of engineered immune cells differ by their cell sources, cell types, expressed therapeutic proteins (e.g., recognition molecule, COR and/or bNAb), and/or promoters, etc.

At various points during preparation of a composition, it can be necessary or beneficial to cryopreserve a cell. The terms “frozen/freezing” and “cryopreserved/cryopreserving” can be used interchangeably. Freezing includes freeze-drying.

In some embodiments, cells can be harvested from a culture medium, and washed and concentrated into a carrier in a therapeutically effective amount. Exemplary carriers include saline, buffered saline, physiological saline, water, Hanks' solution, Ringer's solution, Nonnosol-R (Abbott Labs), Plasma-Lyte A(R) (Baxter Laboratories, Inc., Morton Grove, Ill.), glycerol, ethanol, and combinations thereof.

In some embodiments, carriers can be supplemented with human serum albumin (HSA) or other human serum components or fetal bovine serum. In particular embodiments, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients, which can range in function from a bulking agent to an additive, which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, compositions can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within compositions can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells, including any values and ranges in between these values.

In compositions and formulations disclosed herein, cells are generally in a volume of a liter or less, 500 ml or less, 250 ml or less or 100 ml or less. Hence the density of administered cells is typically greater than 10⁴ cells/ml, 10⁷ cells/ml or 10⁸ cells/ml.

Also provided herein are nucleic acid compositions (such as pharmaceutical compositions, also referred to herein as formulations) comprising any of the nucleic acids encoding a recognition molecule (or one or more portions thereof), optional COR and/or optional bNAb described herein. In some embodiments, the nucleic acid composition is a pharmaceutical composition. In some embodiments, the nucleic acid composition further comprises any of an isotonizing agent, an excipient, a diluent, a thickener, a stabilizer, a buffer, and/or a preservative; and/or an aqueous vehicle, such as purified water, an aqueous sugar solution, a buffer solution, physiological saline, an aqueous polymer solution, or RNase free water. The amounts of such additives and aqueous vehicles to be added can be suitably selected according to the form of use of the nucleic acid composition.

The compositions and formulations disclosed herein can be prepared for administration by, for example, injection, infusion, perfusion, or lavage. The compositions and formulations can further be formulated for bone marrow, intravenous, intradermal, intraarterial, intranodal, intralymphatic, intraperitoneal, intralesional, intraprostatic, intravaginal, intrarectal, topical, intrathecal, intratumoral, intramuscular, intravesicular, and/or subcutaneous injection.

The formulations to be used for in vivo administration must be sterile. This is readily accomplished by, e.g., filtration through sterile filtration membranes.

### Excipient

The pharmaceutical compositions of the present application are useful for therapeutic purposes. Thus, different from other compositions comprising engineered immune cells, such as production cells that express the recognition molecule, optionally COR, and/or optionally bNAb, the pharmaceutical compositions of the present application comprises a pharmaceutically acceptable excipient suitable for administration to an individual.

Suitable pharmaceutically acceptable excipient may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, the pharmaceutically acceptable excipient comprises autologous serum. In some embodiments, the pharmaceutically acceptable excipient comprises human serum. In some embodiments, the pharmaceutically acceptable excipient is non-toxic, biocompatible, non-immunogenic, biodegradable, and can avoid recognition by the host's defense mechanism. The excipient may also contain adjuvants such as preserving stabilizing, wetting, emulsifying agents and the like. In some embodiments, the pharmaceutically acceptable excipient enhances the stability of the engineered immune cell or the antibody or other therapeutic proteins secreted thereof. In some embodiments, the pharmaceutically acceptable excipient reduces aggregation of the antibody or other therapeutic proteins secreted by the engineered immune cell. The final form may be sterile and may also be able to pass readily through an injection device such as a hollow needle. The proper viscosity may be achieved and maintained by the proper choice of excipients.

In some embodiments, the pharmaceutical composition is formulated to have a pH in the range of about 4.5 to about 9.0, including for example pH ranges of about any one of 5.0 to about 8.0, about 6.5 to about 7.5, or about 6.5 to about 7.0. In some embodiments, the pharmaceutical composition can also be made to be isotonic with blood by the addition of a suitable tonicity modifier, such as glycerol.

In some embodiments, the pharmaceutical composition is suitable for administration to a human. In some embodiments, the pharmaceutical composition is suitable for administration to a human by parenteral administration. Formulations suitable for parenteral administration include aqueous and non-aqueous, isotonic sterile injection solutions, which can contain antioxidants, buffers, bacteriostats, and solutes that render the formulation compatible with the blood of the intended recipient, and aqueous and non-aqueous sterile suspensions that can include suspending agents, solubilizers, thickening agents, stabilizing agents, and preservatives. The formulations can be presented in unit-dose or multi-dose sealed containers, such as ampules and vials, and can be stored in a condition requiring only the addition of the sterile liquid excipient methods of treatment, methods of administration, and dosage regimens described herein (i.e., water) for injection, immediately prior to use. In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

In some embodiments, the pharmaceutical composition is formulated for intravenous administration. In some embodiments, the pharmaceutical composition is formulated for subcutaneous administration. In some embodiments, the pharmaceutical composition is formulated for local administration to a tumor site. In some embodiments, the pharmaceutical composition is formulated for intratumoral injection.

In some embodiments, the pharmaceutical composition must meet certain standards for administration to an individual. For example, the United States Food and Drug Administration has issued regulatory guidelines setting standards for cell-based immunotherapeutic products, including 21 CFR 610 and 21 CFR 610.13. Methods are known in the art to assess the appearance, identity, purity, safety, and/or potency of pharmaceutical compositions. In some embodiments, the pharmaceutical composition is substantially free of extraneous protein capable of producing allergenic effects, such as proteins of an animal source used in cell culture other than the engineered mammalian immune cells. In some embodiments, “substantially free” is less than about any of 10%, 5%, 1%, 0.1%, 0.01%, 0.001%, 1 ppm or less of total volume or weight of the pharmaceutical composition. In some embodiments, the pharmaceutical composition is prepared in a GMP-level workshop. In some embodiments, the pharmaceutical composition comprises less than about 5 EU/kg body weight/hr of endotoxin for parenteral administration. In some embodiments, at least about 70% of the engineered immune cells in the pharmaceutical composition are alive for intravenous administration. In some embodiments, the pharmaceutical composition has a “no growth” result when assessed using a 14-day direct inoculation test method as described in the United States Pharmacopoeia (USP). In some embodiments, prior to administration of the pharmaceutical composition, a sample including both the engineered immune cells and the pharmaceutically acceptable excipient should be taken for sterility testing approximately about 48-72 hours prior to the final harvest (or coincident with the last re-feeding of the culture). In some embodiments, the pharmaceutical composition is free of mycoplasma contamination. In some embodiments, the pharmaceutical composition is free of detectable microbial agents. In some embodiments, the pharmaceutical composition is free of communicable disease agents, such as HIV type I, HIV type II, HBV, HCV, Human T-lymphotropic virus, type I; and Human T-lymphotropic virus, type II.

### Methods of Treating Diseases Using Engineered Immune Cells

The present application further provides methods of administering the engineered immune cells to treat diseases, including, but not limited to, infectious diseases, EBV positive T cell lymphoproliferative disorder, T-cell prolymphocytic leukemia, EBV-positive T cell lymphoproliferative disorders, adult T-cell leukemia/lymphoma, mycosis fungoides/sezary syndrome, primary cutaneous T-cell lymphoproliferative disorders, peripheral T-cell lymphoma (not otherwise specified), angioimmunoblastic T-cell lymphoma, and anaplastic large cell lymphoma, and autoimmune diseases.

Engineered immune cells containing distal portion-recognition molecules are particularly suitable for autologous therapies. In some embodiments, autologous lymphocyte infusion is used in the treatment. Autologous PBMCs are collected from a patient in need of treatment and T cells are activated and expanded using the methods described herein and known in the art and then infused back into the patient. In some embodiments, administration of the engineered immune cells results in depletion (for example about 70%, 80%, 90%, 99% or more reduction, or complete elimination) of the engineered immune cells comprising the distal portion-recognition molecule in the individual.

Engineered immune cells containing proximal portion-recognition molecules are particularly suitable for allogeneic therapies. In some embodiments, administration of the engineered immune cells results in no more than about 50% (such as no more than about any of 40%, 30%, 20%, 10%, or 5%) reduction of the engineered immune cells comprising the proximal portion-recognition molecules in the individual.

The engineered immune cells can undergo robust in vivo expansion and can establish target antigen (e.g., CD4 or CD22)-specific memory cells that persist at high levels for an extended period of time in blood and bone marrow. In some embodiments, the engineered immune cells infused into a patient can deplete cancer or virally-infected cells. In some embodiments, the engineered immune cells infused into a patient can eliminate cancer or virally-infected cells. Viral infection treatments can be evaluated, for example, by viral load, duration of survival, quality of life, protein expression and/or activity.

The engineered immune cells of the present application in some embodiments can be administered to individuals (e.g., mammals such as humans) to treat a cancer, for example T cell lymphoma, leukemia, B-cell precursor acute lymphoblastic leukemia (ALL), and B-cell lymphoma. The present application thus in some embodiments provides a method for treating a cancer in an individual comprising administering to the individual an effective amount of a composition (such as a pharmaceutical composition) comprising engineered immune cells according to any one of the embodiments described herein. In some embodiments, cancer is T cell lymphoma.

In some embodiments, the methods of treating a cancer described herein further comprises administering to the individual a second anti-cancer agent. Suitable anti-cancer agents include, but are not limited to, CD70 targeting drugs, TRBC1, CD30 targeting drugs, CD37 targeting drugs, CCR4 targeting drugs, CHOP (cyclophosphamide, doxorubicin, vincristine and prednisone), CHOEP (cyclophosphamide, doxorubicin, vincristine, etoposide and prednisone), EPOCH (etoposide, vincristine, doxorubicin, cyclophosphamide and prednisone), Hyper-CVAD (cyclophosphamide, vincristine, doxorubicin, and dexamethasone), HDAC inhibitors, CD52 antibody Belinostat, Bendamustine, BL-8040, Bortezomib, CPI-613, Mogamulizumab, Nelarabine, Nivolumab, Romidepsin and Ruxolitinib. In some embodiments, the second agent is an immune checkpoint inhibitor (e.g., an anti-CTLA4 antibody, an anti-PD1 antibody, or an anti-PD-L1 antibody). In some embodiments, the second anti-cancer agent is administered simultaneously with the engineered immune cells. In some embodiments, the second anti-cancer agent is administered sequentially with (e.g., prior to or after) the administration of the engineered immune cells. In some embodiments, the engineered immune cell compositions of the invention are administered in combination with a second, third, or fourth agent (including, e.g., an antineoplastic agent, a growth inhibitory agent, a cytotoxic agent, or a chemotherapeutic agent) to treat diseases or disorders involving target antigen expression.

The engineered immune cells of the present application can also be administered to individuals (e.g., mammals such as humans) to treat an infectious disease, for example HIV. The present application thus in some embodiments provides a method for treating an infectious disease in an individual comprising administering to the individual an effective amount of a composition (such as a pharmaceutical composition) comprising engineered immune cells according to any one of the embodiments described herein. In some embodiments, the viral infection is caused by a virus selected from, for example, Human T cell leukemia virus (HTLV) and HIV (Human immunodeficiency virus).

In some embodiments, methods of treating HIV are provided, which comprise administering any of the engineered immune cells described herein. There are two subtypes of HIV: HIV-1 and HIV-2. HIV-1 is the cause of the global pandemic and is a virus with both high virulence and high infectivity. HIV-2, however, is prevalent only in West Africa and is neither as virulent nor as infectious as HIV-1. The differences in virulence and infectivity between HIV-1 and HIV-2 infections may be rooted in the stronger immune response mounted against viral proteins in HIV-2 infections, leading to more efficient control in affected individuals (Leligdowicz, A. et al. (2007) J. Clin. Invest. 117(10):3067-3074). This may also be a controlling reason for the global spread of HIV-1 and the limited geographic prevalence of HIV-2.

Although HIV-2 infections are better controlled than HIV-1 infections, HIV-2-affected individuals still benefit from treatment. In some embodiments, the engineered immune cells are used for treating HIV-1 infections. In other embodiments, the engineered immune cells are used for treating HIV-2 infections. In some embodiments, the engineered immune cells are used for treating HIV-1 and HIV-2 infections.

In some embodiments, the methods of treating an infectious disease described herein further comprises administering to the individual a second anti-infectious disease agent. Suitable anti-infectious disease agents include, but are not limited to, anti-retroviral drugs, broad neutralization antibodies, toll-like receptor agonists, latency reactivation agents, CCR5 antagonists, immune stimulators (e.g., TLR ligands), vaccines, nucleoside reverse transcriptase inhibitors, nucleotide reverse transcriptase inhibitors, non-nucleoside reverse transcriptase inhibitors, HIV protease inhibitors, and fusion inhibitors. In some embodiments, the second anti-infectious agent is administered simultaneously with the engineered immune cells. In some embodiments, the second anti-infectious agent is administered sequentially with (e.g., prior to or after) the administration of the engineered immune cells.

In some embodiments, the individual is a mammal (e.g., human, non-human primate, rat, mouse, cow, horse, pig, sheep, goat, dog, cat, etc.). In some embodiments, the individual is a human. In some embodiments, the individual is a clinical patient, a clinical trial volunteer, an experimental animal, etc. In some embodiments, the individual is younger than about 60 years old (including for example younger than about any of 50, 40, 30, 25, 20, 15, or 10 years old). In some embodiments, the individual is older than about 60 years old (including for example older than about any of 70, 80, 90, or 100 years old). In some embodiments, the individual is diagnosed with or environmentally or genetically prone to one or more of the diseases or disorders described herein (such as cancer or viral infection). In some embodiments, the individual has one or more risk factors associated with one or more diseases or disorders described herein.

In some embodiments, the pharmaceutical composition is administered at a dose of at least about any of 10⁴, 10⁵, 10⁶, 10⁷, 10⁸, or 10⁹ cells/kg of body weight. In some embodiments, the pharmaceutical composition is administered at a dose of any of about 10⁴ to about 10⁵, about 10⁵ to about 10⁶, about 10⁶ to about 10⁷, about 10⁷ to about 10⁸, about 10⁸ to about 10⁹, about 10⁴ to about 10⁹, about 10⁴ to about 10⁶, about 10⁶ to about 10⁸, or about 10⁵ to about 10⁷ cells/kg of body weight.

In some embodiments, wherein more than one type of engineered immune cells are administered, the different types of engineered immune cells may be administered to the individual simultaneously, such as in a single composition, or sequentially in any suitable order.

In some embodiments, the pharmaceutical composition is administered for a single time. In some embodiments, the pharmaceutical composition is administered for multiple times (such as any of 2, 3, 4, 5, 6, or more times). In some embodiments, the pharmaceutical composition is administered once per week, once 2 weeks, once 3 weeks, once 4 weeks, once per month, once per 2 months, once per 3 months, once per 4 months, once per 5 months, once per 6 months, once per 7 months, once per 8 months, once per 9 months, or once per year. In some embodiments, the interval between administrations is about any one of 1 week to 2 weeks, 2 weeks to 1 month, 2 weeks to 2 months, 1 month to 2 months, 1 month to 3 months, 3 months to 6 months, or 6 months to a year. The optimal dosage and treatment regime for a particular patient can readily be determined by one skilled in the art of medicine by monitoring the patient for signs of disease and adjusting the treatment accordingly.

Thus, for example, in some embodiments, there is provided a method of treating an individual having a cancer, comprising administering to the individual an engineered immune cell (such as cytotoxic T cell, NK cell, or γδT cell) comprising on its surface a recognition molecule that comprises a binding moiety specifically binding to a target molecule on the surface of a target cell, wherein the target molecule comprises an extracellular domain (such as an extracellular domain that is at least about 175 amino acids long), wherein the binding moiety specifically binds to a distal portion of the extracellular domain, wherein the immune cell is capable of killing a target cell that comprises on its surface the target molecule, and wherein the immune cell is capable of killing a target cell that comprises on its surface both the target molecule and the recognition molecule, and wherein the engineered immune cells are autologous to the individual. In some embodiments, the recognition molecule is an immune cell receptor, such as a CAR or a cTCR.

In some embodiments, there is provided a method of treating an individual having a cancer (e.g., B-cell related cancer, for example B-cell precursor acute lymphoblastic leukemia or B-cell lymphoma), comprising administering to the individual an effective amount of an engineered immune cells (or pharmaceutical composition comprising engineered immune cells) comprising an anti-CD22 immune cell receptor, wherein the anti-CD22 immune cell receptor comprises an extracellular domain comprising a CD22 binding moiety that specifically binds to an epitope within D1-4 of CD22, a transmembrane domain, and an intracellular signaling domain, and wherein the engineered immune cells are autologous to the individual. In some embodiments, the anti-CD22 immune cell receptor is an anti-CD22 D1-4 CAR. In some embodiments, the anti-CD22 immune cell receptor is an anti-CD22 D1-4 cTCR. In some embodiments, the cancer is CD22+. In some embodiments, the cancer is B-cell precursor acute lymphoblastic leukemia. In some embodiments, the cancer is B-cell lymphoma. In some embodiments, the method further comprises administering to the individual a second anti-cancer agent, for example an anti-cancer agent selected from the group consisting of CD70 targeting drugs, TRBC1, CD30 targeting drugs, CD37 targeting drugs CCR4 targeting drugs, CHOP (cyclophosphamide, doxorubicin, vincristine and prednisone), CHOEP (cyclophosphamide, doxorubicin, vincristine, etoposide and prednisone), EPOCH (etoposide, vincristine, doxorubicin, cyclophosphamide and prednisone), Hyper-CVAD (cyclophosphamide, vincristine, doxorubicin, and dexamethasone), HDAC inhibitors, CD52 antibody Belinostat, Bendamustine, BL-8040, Bortezomib, CPI-613, Mogamulizumab, Nelarabine, Nivolumab, Romidepsin and Ruxolitinib. In some embodiments, the second anti-cancer agent is a checkpoint inhibitor (such as anti-CTLA4, anti-PD1, and anti-PD-L1). In some embodiments, the method further comprises obtaining immune cells from the individual. In some embodiments, the method further comprises introducing one or more nucleic acids encoding the anti-CD22 D1-4 immune cell receptor into the immune cells to generate the engineered immune cells comprising the anti-CD22 D1-4 immune cell receptor. In some embodiments, the administration of the engineered immune cells results in reduction (for example about 70%, 80%, 90%, 99% or more reduction, or complete elimination) of the engineered immune cells comprising the anti-CD22 D1-4 immune cell receptor in the individual.

In some embodiments, there is provided a method of reducing the number of target cells expressing a target molecule on its surface (such as cancer cells), comprising contacting the target cells with an effective amount of an engineered immune cells (such as cytotoxic T cell, NK cell, or γδT cell) comprising on its surface a recognition molecule that comprises a binding moiety specifically binding to the target molecule on the surface of the target cell, wherein the target molecule comprises an extracellular domain (such as an extracellular domain that is at least about 175 amino acids long), wherein the binding moiety specifically binds to a distal portion of the extracellular domain, wherein the immune cell is capable of killing a target cell that comprises on its surface the target molecule, and wherein the immune cell is capable of killing a target cell that comprises on its surface both the target molecule and the recognition molecule, and wherein the engineered immune cells and the target cells are derived from the same individual. In some embodiments, the recognition molecule is an immune cell receptor, such as a CAR or a cTCR.

In some embodiments, there is provided a method of reducing the number of CD22+ cells (e.g., B-cell related CD22+ cancer cells, for example B-cell precursor acute lymphoblastic leukemia cells or B-cell lymphoma cells), comprising contacting the CD22+ cells with an effective amount of an engineered immune cells (or pharmaceutical composition comprising engineered immune cells) comprising an anti-CD22 immune cell receptor, wherein the anti-CD22 immune cell receptor comprises an extracellular domain comprising a CD2 binding moiety that specifically binds to an epitope within D1-4 of CD22, a transmembrane domain, and an intracellular signaling domain, and wherein the engineered immune cells and the CD22+ cells are derived from the same individual. In some embodiments, the anti-CD22 immune cell receptor is an anti-CD22 D1-4 CAR. In some embodiments, the anti-CD22 immune cell receptor is an anti-CD22 D1-4 cTCR.

In some embodiments, there is provided a method of treating an individual having a cancer, comprising administering to the individual an engineered immune cell (such as cytotoxic T cell, NK cell, or γδT cell) comprising on its surface a recognition molecule that comprises a binding moiety specifically binding to a target molecule on the surface of a target cell, wherein the target molecule comprises an extracellular domain (such as an extracellular domain that is at least about 175 amino acids long), wherein the binding moiety specifically binds to a proximal portion of the extracellular domain, wherein the engineered immune cell is capable of killing a target cell that comprises on its surface the target molecule, wherein the engineered immune cell has no or reduced capability of killing a target cell comprising on its surface both the target molecule and the recognition molecule, and wherein the engineered immune cells are allogeneic to the individual. In some embodiments, the recognition molecule is an immune cell receptor, such as a CAR or a cTCR.

In some embodiments, there is provided a method of treating an individual having a cancer (e.g., B-cell related cancer, for example B-cell precursor acute lymphoblastic leukemia or B-cell lymphoma), comprising administering to the individual an effective amount of an engineered immune cells (or pharmaceutical composition comprising engineered immune cells) comprising an anti-CD22 immune cell receptor, wherein the anti-CD22 immune cell receptor comprises an extracellular domain comprising a CD22 binding moiety that specifically binds to an epitope within D5-7 of CD22, a transmembrane domain, and an intracellular signaling domain, and wherein the engineered immune cells are allogeneic to the individual. In some embodiments, the anti-CD22 immune cell receptor is an anti-CD22 D5-7 CAR. In some embodiments, the anti-CD22 immune cell receptor is an anti-CD22 D5-7 cTCR. In some embodiments, the cancer is CD22+. In some embodiments, the cancer is B cell lymphoma. In some embodiments, the method further comprises administering to the individual a second anti-cancer agent, for example an anti-cancer agent selected from the group consisting of CD70 targeting drugs, TRBC1, CD30 targeting drugs, CD37 targeting drugs and CCR4 targeting drugs. In some embodiments, the second anti-cancer agent is a checkpoint inhibitor (such as anti-CTLA4, anti-PD1, and anti-PD-L1). In some embodiments, the method further comprises obtaining immune cells from a donor individual. In some embodiments, the method further comprises introducing one or more nucleic acids encoding the anti-CD22 D5-7 immune cell receptor into the immune cells to generate the engineered immune cells comprising the anti-CD22 D5-7 immune cell receptor. In some embodiments, the administration of the engineered immune cells result in no more than about 50% (such as no more than about any of 40%, 30%, 20%, 10%, or 5%) reduction of the engineered immune cells comprising the anti-CD22 D5-7 immune cell receptor in the individual. In some embodiments, the engineered immune cells are modified to inactivate components of TCR involved in MHC recognition. In some embodiments, the engineered immune cells do not cause GvHD.

In some embodiments, there is provided a method of reducing the number of target cells expressing a target molecule on its surface (such as cancer cells), comprising contacting the target cells with an effective amount of an engineered immune cells (such as cytotoxic T cell, NK cell, or γδT cell) comprising on its surface a recognition molecule that comprises a binding moiety specifically binding to the target molecule on the surface of the target cell, wherein the target molecule comprises an extracellular domain (such as an extracellular domain that is at least about 175 amino acids long), wherein the binding moiety specifically binds to a proximal portion of the extracellular domain, wherein the immune cell is capable of killing a target cell that comprises on its surface the target molecule, wherein the engineered immune cell has no or reduced capability of killing a target cell comprising on its surface both the target molecule and the recognition molecule, and wherein the engineered immune cells and the target cells are derived from a different individual. In some embodiments, the recognition molecule is an immune cell receptor, such as a CAR or a cTCR.

In some embodiments, there is provided a method of reducing the number of CD22+ cells (e.g., B-cell related CD22+ cancer cells, for example B-cell precursor acute lymphoblastic leukemia cells or B-cell lymphoma cells), comprising contacting the CD22+ cells with an effective amount of an engineered immune cells (or pharmaceutical composition comprising engineered immune cells) comprising an anti-CD22 immune cell receptor, wherein the anti-CD22 immune cell receptor comprises an extracellular domain comprising a CD2 binding moiety that specifically binds to an epitope within D5-7 of CD22, a transmembrane domain, and an intracellular signaling domain, and wherein the engineered immune cells and the CD22+ cells are derived from a different individual. In some embodiments, the anti-CD22 immune cell receptor is an anti-CD22 D5-7 CAR. In some embodiments, the anti-CD22 immune cell receptor is an anti-CD22 D5-7 cTCR.

Thus, for example, in some embodiments, there is provided a method of treating an individual having an infectious disease (such as HIV), comprising administering to the individual an engineered immune cell (such as cytotoxic T cell, NK cell, or γδT cell) comprising on its surface a recognition molecule that comprises a binding moiety specifically binding to a target molecule on the surface of a target cell, wherein the target molecule comprises an extracellular domain (such as an extracellular domain that is at least about 175 amino acids long), wherein the binding moiety specifically binds to a distal portion of the extracellular domain, wherein the immune cell is capable of killing a target cell that comprises on its surface the target molecule, and wherein the immune cell is capable of killing a target cell that comprises on its surface both the target molecule and the recognition molecule, and wherein the engineered immune cells are autologous to the individual. In some embodiments, the recognition molecule is an immune cell receptor, such as a CAR or a cTCR.

In some embodiments, there is provided a method of treating an individual having a an infectious disease (such as HIV), comprising administering to the individual an engineered immune cell (such as cytotoxic T cell, NK cell, or γδT cell) comprising on its surface a recognition molecule that comprises a binding moiety specifically binding to a target molecule on the surface of a target cell, wherein the target molecule comprises an extracellular domain (such as an extracellular domain that is at least about 175 amino acids long), wherein the binding moiety specifically binds to a proximal portion of the extracellular domain, wherein the engineered immune cell is capable of killing a target cell that comprises on its surface the target molecule, wherein the engineered immune cell has no or reduced capability of killing a target cell comprising on its surface both the target molecule and the recognition molecule, and wherein the engineered immune cells are allogeneic to the individual. In some embodiments, the recognition molecule is an immune cell receptor, such as a CAR or a cTCR.

### Articles of Manufacture and Kits

In some embodiments of the present application, there is provided an article of manufacture containing materials useful for the treatment of a cancer (e.g., B-cell related cancer) or an infectious disease such as viral infection (e.g., infection by HIV). The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example, the container may be an intravenous solution bag or a vial having a stopper pierceable by a hypodermic injection needle). At least one active agent in the composition is an engineered immune cell presenting on its surface a recognition molecule described herein. The label or package insert indicates that the composition is used for treating a particular disease or condition. The label or package insert will further comprise instructions for administering the engineered immune cell composition to the patient. Articles of manufacture and kits comprising combination therapies described herein are also contemplated.

Package insert refers to instructions customarily included in commercial packages of therapeutic products that contain information about the indications, usage, dosage, administration, contraindications and/or warnings concerning the use of such therapeutic products. In other embodiments, the package insert indicates that the composition is used for treating a target antigen-positive viral infection (for example, infection by HIV) or cancer (e.g., B-cell related cancer).

Additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a target antigen-positive disease or disorder described herein, optionally in combination with the articles of manufacture. Kits of the invention include one or more containers comprising an engineered immune cell composition (or unit dosage form and/or article of manufacture), and in some embodiments, further comprise another agent (such as the agents described herein) and/or instructions for use in accordance with any of the methods described herein. The kit may further comprise a description of selection of individuals suitable for treatment. Instructions supplied in the kits of the present application are typically written instructions on a label or package insert (e.g., a paper sheet included in the kit), but machine-readable instructions (e.g., instructions carried on a magnetic or optical storage disk) are also acceptable.

Those skilled in the art will recognize that several embodiments are possible within the scope and spirit of this invention. The invention will now be described in greater detail by reference to the following non-limiting exemplary embodiments and examples. The following exemplary embodiments and examples further illustrate the invention but, of course, should not be construed as in any way limiting its scope.

### Exemplary Embodiments

The present application provides the following embodiments:
1. An engineered immune cell comprising on its surface a recognition molecule that comprises a binding moiety specifically binding to a target molecule on the surface of a target cell, wherein the target molecule comprises an extracellular domain, wherein the binding moiety specifically binds to a distal portion of the extracellular domain, wherein the immune cell is capable of killing a target cell that comprises on its surface the target molecule, and wherein the immune cell is capable of killing a target cell that comprises on its surface both the target molecule and the recognition molecule.
2. The engineered immune cell of embodiment 1, wherein the recognition molecule comprises the binding moiety, a transmembrane domain, and an intracellular signaling domain.
3. The engineered immune cell of embodiment 1 or 2, wherein the binding moiety is a single domain antibody (sdAb), an scFv, a Fab′, a (Fab′)₂, an Fv, or a peptide ligand.
4. The engineered immune cell of any one of embodiments 1-3, wherein the distance from the distal portion of the extracellular domain to the membrane of the target cell is more than about 0.5 times (e.g., more than about 1 time, 1.5 times, 2 times, or more) of the distance from the binding moiety to the membrane of engineered immune cell.
5. The engineered immune cell of any one of embodiments 1-4, wherein the extracellular domain of the target molecule is at least about 175 amino acids long.
6. The engineered immune cell of any one of embodiments 1-5, wherein:
   (i) the binding moiety binds to a region in the extracellular domain that is about 50 amino acids or more away from the C-terminus of the extracellular domain;
   (ii) the binding moiety binds to a region in the extracellular domain that is about 80 amino acids or more away from the C-terminus of the extracellular domain; and/or
   (iii) the binding moiety binds to a region that is within about 120 (e.g., about 80) amino acids from the N-terminus of the extracellular domain.
7. The engineered immune cell of any one of embodiments 1-6, wherein the distal portion of the extracellular domain is at least about 30 Å (e.g., at least about 40, 60, 90, 120 or more A) away from the membrane of the target cell.
8. The engineered immune cell of any one of embodiments 1-7, wherein the extracellular domain of the target molecule comprises three or more Ig-like domains.
9. The engineered immune cell of embodiment 8, wherein the binding moiety binds to a region outside the first two (e.g., outside the first three) Ig-like domains from the C-terminal end of the extracellular domain.
10. The engineered immune cell of embodiment 8 or 9, wherein the binding moiety binds to a region within the first four (e.g., within the first) Ig-like domain at the N-terminal end of the extracellular domain.
11. The engineered immune cell of any one of embodiments 1-10, wherein the target molecule is a transmembrane receptor.
12. The engineered immune cell of embodiment 11, wherein the target molecule is selected from the group consisting of CD22, CD4, CD21 (CR2), CD30, ROR1, CD5, and CD20.
13. The engineered immune cell of embodiment 12, wherein the target molecule is CD22.
14. The engineered immune cell of embodiment 13, wherein the binding moiety competes for binding with a reference antibody that specifically binds to an epitope within Domains 1-4 of CD22 (“anti-CD22 D1-4 antibody”).
15. The engineered immune cell of embodiment 13 or 14, wherein the binding moiety binds to an epitope in Domains 1-4 of CD22 that overlaps with the binding epitope of a reference anti-CD22 D1-4 antibody.
16. The engineered immune cell of any one of embodiments 13-15, wherein the binding moiety comprises the same heavy chain and light chain CDR sequences as those of a reference anti-CD22 D1-4 antibody.
17. The engineered immune cell of embodiment 16, wherein the binding moiety comprises the same heavy chain variable domain (VH) and light chain variable domain (VL) sequences as those of a reference anti-CD22 D1-4 antibody.
18. The engineered immune cell of any one of embodiments 14-17, wherein the reference anti-CD22 D1-4 antibody comprises a heavy chain CDR1 (HC-CDR1) comprising the amino acid sequence of SEQ ID NO: 67, a heavy chain CDR2 (HC-CDR2) comprising the amino acid sequence of SEQ ID NO: 68, a heavy chain CDR3 (HC-CDR3) comprising the amino acid sequence of SEQ ID NO: 69, a light chain CDR1 (LC-CDR1) comprising the amino acid sequence of SEQ ID NO: 70, a light chain CDR2 (LC-CDR2) comprising the amino acid sequence of SEQ ID NO: 71, and a light chain CDR3 (LC-CDR3) comprising the amino acid sequence of SEQ ID NO: 72.
19. The engineered immune cell of any one of embodiments 14-18, wherein the reference anti-CD22 D1-4 antibody comprises a VH comprising the amino acid sequence of SEQ ID NO: 73 and a VL comprising the amino acid sequence of SEQ ID NO: 74.
20. The engineered immune cell of any one of embodiments 1-19, wherein the engineered immune cell is capable of killing a target cell that comprises on its surface both the target molecule and the recognition molecule by at least 3 fold as compared to an engineered immune cell comprising on its surface a recognition molecule comprising a binding moiety that binds to a proximal portion of the extracellular domain of the target molecule.
21. An engineered immune cell comprising on its surface a recognition molecule that comprises a binding moiety specifically binding to a target molecule on the surface of a target cell, wherein the target molecule comprises an extracellular domain, wherein the binding moiety specifically binds to a proximal portion of the extracellular domain, wherein the engineered immune cell is capable of killing a target cell that comprises on its surface the target molecule, and wherein the engineered immune cell has no or reduced capability of killing a target cell comprising on its surface both the target molecule and the recognition molecule.
22. The engineered immune cell of embodiment 21, wherein the recognition molecule comprises the binding moiety, a transmembrane domain, and an intracellular signaling domain.
23. The engineered immune cell of embodiment 21 or 22, wherein the binding moiety is an sdAb, an scFv, a Fab′, a (Fab′)2, an Fv, or a peptide ligand.
24. The engineered immune cell of any one of embodiments 21-23, wherein the distance from the proximal portion of the extracellular domain to the membrane of the target cell is no more than about 2 times (e.g., no more than about 1.5 times, or no more than about 1 time) of the distance from the binding moiety to the membrane of engineered immune cell.
25. The engineered immune cell of any one of embodiments 21-24, wherein the extracellular domain of the target molecule is at least about 175 amino acids long.
26. The engineered immune cell of any one of embodiments 21-25, wherein:
   (i) the binding moiety binds outside of a region that is about 80 amino acids or more away from the N-terminus of the extracellular domain;
   (ii) the binding moiety binds to a region in the extracellular domain that is within about 120 (e.g., about 102) amino acids from the C-terminus of the extracellular domain; and/or
   (iii) the binding moiety binds to a region in the extracellular domain that is within about 50 amino acids from the C-terminus of the extracellular domain.
27. The engineered immune cell of any one of embodiments 21-26, wherein the proximal portion of the extracellular domain is no more than about 120 Å (e.g., no more than about 100, 90, 80, 70 or 60 Å) from the membrane of the target cell.
28. The engineered immune cell of any one of embodiments 21-27, wherein the extracellular domain of the target molecule comprises two or more Ig-like domains.
29. The engineered immune cell of embodiment 28, wherein the binding moiety binds to a region outside the first (e.g., outside the first four) Ig-like domain at the N-terminal end of the extracellular domain.
30. The engineered immune cell of embodiment 29, wherein the binding moiety binds to a region within the first three (e.g., within the first two) Ig-like domains from the C-terminal end of the extracellular domain.
31. The engineered immune cell of any one of embodiments 21-30, wherein the target molecule is a transmembrane receptor.
32. The engineered immune cell of embodiment 31, wherein the target molecule is selected from the group consisting of CD22, CD4, CD21 (CR2), CD30, ROR1, CD5, and CD20.
33. The engineered immune cell of embodiment 32, wherein the target molecule is CD22.
34. The engineered immune cell of embodiment 33, wherein the binding moiety competes for binding with a reference antibody that specifically binds to an epitope within Domains 5-7 of CD22 (“anti-CD22 D5-7 antibody”).
35. The engineered immune cell of embodiment 33 or 34, wherein the binding moiety binds to an epitope in Domains 5-7 of CD22 that overlaps with the binding epitope of a reference anti-CD22 D5-7 antibody.
36. The engineered immune cell of any one of embodiments 33-35, wherein the binding moiety comprises the same heavy chain and light chain CDR sequences as those of a reference anti-CD22 D5-7 antibody.
37. The engineered immune cell of embodiment 36, wherein the binding moiety comprises the same VH and VL sequences as those of a reference anti-CD22 D5-7 antibody.
38. The engineered immune cell of any one of embodiments 34-37, wherein the reference anti-CD22 D5-7 antibody comprises a HC-CDR1 comprising the amino acid sequence of SEQ ID NO: 76, a HC-CDR2 comprising the amino acid sequence of SEQ ID NO: 77, a HC-CDR3 comprising the amino acid sequence of SEQ ID NO: 78, a LC-CDR1 comprising the amino acid sequence of SEQ ID NO: 79, a LC-CDR2 comprising the amino acid sequence of SEQ ID NO: 80, and a LC-CDR3 comprising the amino acid sequence of SEQ ID NO: 81.
39. The engineered immune cell of any one of embodiments 35-38, wherein the reference anti-CD22 D5-7 antibody comprises a VH comprising the amino acid sequence of SEQ ID NO: 82 and a VL comprising the amino acid sequence of SEQ ID NO: 83.
40. The engineered immune cell of any one of embodiments 21-39, wherein the engineered immune cell kills a target cell that comprises on its surface both the target molecule and the recognition molecule by no more than about 20% as compared to an engineered immune cell comprising on its surface a recognition molecule comprising a binding moiety that binds to a distal end of the extracellular domain of the target molecule.
41. The engineered immune cell of any one of embodiments 1-40, wherein the recognition molecule is monospecific.
42. The engineered immune cell of any one of embodiments 1-40, wherein the recognition molecule is multispecific.
43. The engineered immune cell of embodiment 42, wherein the recognition molecule comprises a second binding moiety specifically recognizing a second target molecule.
44. The engineered immune cell of embodiment 43, wherein the second binding moiety is an sdAb, an scFv, a Fab′, a (Fab′)₂, an Fv, or a peptide ligand.
45. The engineered immune cell of embodiment 43 or 44, wherein the binding moiety and the second binding moiety are linked in tandem.
46. The engineered immune cell of embodiment 45, wherein the binding moiety is N-terminal to the second binding moiety.
47. The engineered immune cell of embodiment 45, wherein the binding moiety is C-terminal to the second antigen binding moiety.
48. The engineered immune cell of any one of embodiments 43-47, wherein the binding moiety and the second binding moiety are linked via a linker.
49. The engineered immune cell of any one of embodiments 2-48, wherein the binding moiety is fused to the transmembrane domain directly or indirectly.
50. The engineered immune cell of embodiment 49, wherein the binding moiety is non-covalently bound to a polypeptide comprising the transmembrane domain.
51. The engineered immune cell of embodiment 50, wherein the recognition molecule comprises
   i) a first polypeptide comprising the binding moiety and a first member of a binding pair; and
   ii) a second polypeptide comprising a second member of the binding pair, wherein the first member and the second member bind to each other, and wherein the second member is fused to the transmembrane domain directly or indirectly.
52. The engineered immune cell of embodiment 49, wherein the binding moiety is fused to a polypeptide comprising the transmembrane domain.
53. The engineered immune cell of any one of embodiments 1-52, wherein the recognition molecule is a chimeric antigen receptor (“CAR”).
54. The engineered immune cell of embodiment 53, wherein the transmembrane domain is derived from a molecule selected from the group consisting of CD8α, CD4, CD28, 4-1BB, CD80, CD86, CD152 and PD1.
55. The engineered immune cell of embodiment 54, wherein the transmembrane domain is derived from CD8α.
56. The engineered immune cell of any one of embodiments 53-55, wherein the intracellular signaling domain comprises a primary intracellular signaling domain derived from CD3ζ, FcRγ, FcRβ, CD3γ, CD3δ, CD3ε, CD5, CD22, CD79a, CD79b, or CD66d.
57. The engineered immune cell of embodiment 56, wherein the primary intracellular signaling domain is derived from CD3ζ.
58. The engineered immune cell of embodiment any one of embodiments 53-57, wherein the intracellular signaling domain comprises a co-stimulatory signaling domain.
59. The engineered immune cell of embodiment 58, wherein the co-stimulatory signaling domain is derived from a co-stimulatory molecule selected from the group consisting of CD27, CD28, 4-1BB, OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, DAP10, DAP12, CD83, ligands of CD83 and combinations thereof.
60. The engineered immune cell of embodiments 59, wherein the co-stimulatory signaling domain comprises a cytoplasmic domain of 4-1BB.
61. The engineered immune cell of any one of embodiments 53-60, wherein the recognition molecule further comprises a hinge domain located between the C-terminus of the binding moiety and the N-terminus of the transmembrane domain.
62. The engineered immune cell of embodiment 61, wherein the hinge domain is derived from CD8a or IgG4 CH2-CH3.
63. The engineered immune cell of any one of embodiments 1-52, wherein the recognition molecule is a chimeric T cell receptor (“cTCR”).
64. The engineered immune cell of embodiment 63, wherein the transmembrane domain is derived from the transmembrane domain of a TCR subunit selected from the group consisting of TCRα, TCRβ, TCRγ, TCRδ, CD3γ, CD3ε, and CD3δ.
65. The engineered immune cell of embodiment 64, wherein the transmembrane domain is derived from the transmembrane domain of CD3.
66. The engineered immune cell of any one of embodiments 63-65, wherein the intracellular signaling domain is derived from the intracellular signaling domain of a TCR subunit selected from the group consisting of TCRα, TCRβ, TCRγ, TCRδ, CD3γ, CD3ε, and CD3δ.
67. The engineered immune cell of embodiment 66, wherein the intracellular signaling domain is derived from the intracellular signaling domain of CD3.
68. The engineered immune cell of embodiment 66 or 67, wherein the transmembrane domain and intracellular signaling domain of the recognition molecule are derived from the same TCR subunit.
69. The engineered immune cell of any one of embodiments 63-68, wherein the recognition molecule further comprises at least a portion of an extracellular domain of a TCR subunit.
70. The engineered immune cell of embodiment 69, wherein the binding moiety is fused to the N-terminus of CD3F (“eTCR”).
71. The engineered immune cell of any one of embodiments 1-70, wherein the engineered immune cell is a T cell.
72. The engineered immune cell of embodiment 71, wherein the immune cell is selected from the group consisting of a cytotoxic T cell, a helper T cell, a natural killer (NK) cell, a natural killer T (NK-T) cell, and a γδT cell.
73. The engineered immune cell of any one of embodiments 1-72, further comprising a co-receptor.
74. The engineered immune cell of embodiment 73, wherein the co-receptor is a chemokine receptor.
75. The engineered immune cell of any one of embodiments 1-74, wherein the target cell is an immune cell.
76. The engineered immune cell of any one of embodiments 1-74, wherein the target cell is a tumor cell.
77. A pharmaceutical composition comprising the engineered immune cell of any one of embodiments 1-20 and 41-76.
78. A pharmaceutical composition comprising the engineered immune cell of any one of 21-76.
79. A method of treating an individual having a cancer, comprising administering to the individual an effective amount of the pharmaceutical composition of embodiment 77.
80. The method of embodiment 79, wherein the engineered immune cells are autologous to the individual.
81. The method of embodiment 79 or 80, wherein the cancer is selected from the group consisting of T cell lymphoma, leukemia, B-cell precursor acute lymphoblastic leukemia (ALL), and B-cell lymphoma.
82. A method of treating an individual having an infectious disease, comprising administering to the individual an effective amount of the pharmaceutical composition of embodiment 77.
83. The method of embodiment 82, wherein the engineered immune cells are autologous to the individual.
84. The method of embodiment 82 or 83, wherein the infectious disease is an infection by a virus selected from the group consisting of HIV and HTLV.
85. The method of embodiment 84, wherein the infectious disease is HIV.
86. A method of treating an individual having a cancer, comprising administering to the individual an effective amount of the pharmaceutical composition of embodiment 78.
87. The method of embodiment 86, wherein the engineered immune cell is allogeneic to the individual.
88. The method of embodiment 86 or 87, wherein the cancer is selected from the group consisting of T cell lymphoma, leukemia, B-cell precursor acute lymphoblastic leukemia (ALL), and B-cell lymphoma.
89. A method of treating an individual having an infectious disease, comprising administering to the individual an effective amount of the pharmaceutical composition of embodiment 78.
90. The method of embodiment 89, wherein the engineered immune cells are allogeneic to the individual.
91. The method of embodiment 89 or 90, wherein the infectious disease is an infection by a virus selected from the group consisting of HIV and HTLV.
92. The method of embodiment 91, wherein the infectious disease is HIV.
93. A method of making the engineered immune cell of any one of embodiments 1-76, comprising introducing one or more nucleic acids encoding the recognition molecule into an immune cell, thereby obtaining the engineered immune cell.
94. A composition comprising one or more nucleic acids encoding the recognition molecule of the engineered immune cell of any one of embodiments 1-76.

### Examples

### Example 1: Materials and Methods

CAR-T cell construction. Plasmids containing CAR-encoding coding sequences were synthesized in Genscript and cloned into pLVX lentiviral vector. Second generation lentiviruses were packaged in 293T cells. Pan T cells were isolated from human PBMC (Hemacare) and activated in vitro by anti-CD3/anti-CD28 beads (Miltenyi) for 2 days before they were transduced with CAR-coding lentiviruses in the presence of 8 g/ml polybrene. Cells were spinoculated with the lentiviruses at 1000 g at 32° C. for one hour and were cultured in 24-well plates. Old media was removed and fresh media was added one day post the transduction.

CAR-T cell maintenance and phenotyping. CAR-T cells are cultured in AIM-V media (Thermal Fisher Scientific)+5% Fetal Bovine Serum (FBS)+3001U/ml IL-2. CAR+ percentages were detected 4 days post transduction by anti-Fab antibodies (Jackson Laboratories). Cells were also stained with anti-CD4 and anti-CD8 antibodies to characterize the population.

Cell killing assays. T cell leukemia/lymphoma cell lines Sup-T1 and HH, or CFSE labeled human pan T cells were used as target cells. CAR-T cells were used as effector cells. CAR-T cells and target cells were mixed at desired E:T ratios. Cells were co-cultured before they were collected for flow cytometry. Supernatant was also harvested for cytokine detection. Target cell killing was determined by the CFSE positive cell rate or CD4+ positive cell rate.

Domain mapping. Human CD4 protein contains four extracellular immunoglobulin-like domains (D1 to D4) and an intracellular domain (D5). Each human CD4 domain was cloned into a mouse CD4 backbone and replaced the mouse CD4 counter-domain to generate hybrid CD4 proteins. The hybrid CD4 coding sequences were cloned into pcDNA3.4 vector and were transiently expressed in HEK-293 cells. Anti-human CD4 antibodies were used to stain these cells to determine which human CD4 domain will be recognized by each antibody. Data was collected on a BD FACS Celesta flow cytometer and analyzed by Flowjo software.

Epitope binning experiment. The epitope binning experiment was carried out on Biacore instrument. Briefly, the first antibody was fixed on the chip, CD4-Fc protein flew through the chip during the first phase. A secondary antibody was mixed with CD4-Fc protein at 2:1 ratio and flew through the chip during the second phase. The signal was recorded by Biacore.

Antibody blocking assay. Ibalizumab, Tregalizumab and Zanolimumab monoclonal antibodies were manufactured in Genscript and were used as blocking antibodies in the experiment. Effector and CFSE labeled target cells were co-cultured in the absence or presence of the blocking antibodies of 50 nM or 100 nM as indicated in figures. Target cell killing was measured by detecting CFSE by flow cytometry. Different concentrations of antibodies were used as indicated in the figures.

CAR+ Tumor cell killing assay. Human cutaneous T lymphoma cell line HH cells were transduced with anti-CD4 CAR lentiviruses and the CAR+ rate was detected by flow cytometry. 8×10⁴ HH cells or CAR-HH cells were used as target cells and were co-cultured with anti-CD4 CAR-T effector cells or UNT cells at E:T=2:1. After 8 days of co-culture, the CD4+% was detected by flow cytometry.

In vivo efficacy. NOD-Prkdc^{em26Cd52}Il2rg^{em26cd22}/NJuCr mice (NCG) mice were purchased from Nanjing Biomedical Research Institute of Nanjing University and maintained in Genscript model animal facilities. The neonatal NCG mice were transplanted with human hematopoietic stem cells and mice>15 weeks of age were used in the experiments. NCG mice was treated with 3×10⁵ CAR+ anti-CD4 domain 1 CAR-T cells or the same total amount of un-transduced cells as control. At day 18 post treatment, the mice were sacrificed and the splenocytes were stained with anti-human CD45 antibody, anti-human CD4 antibody and anti-human CD8 antibody. Data was collected on a BD FACS celesta flow cytometer and was analyzed by Flowjo software.

### Example 2. Analysis of Anti-CD4 CAR-T Cells

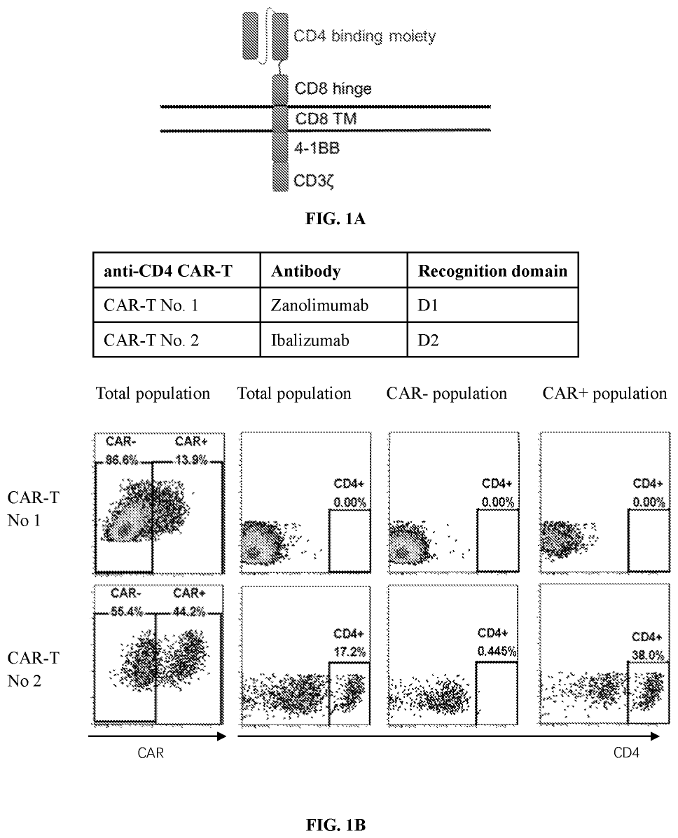

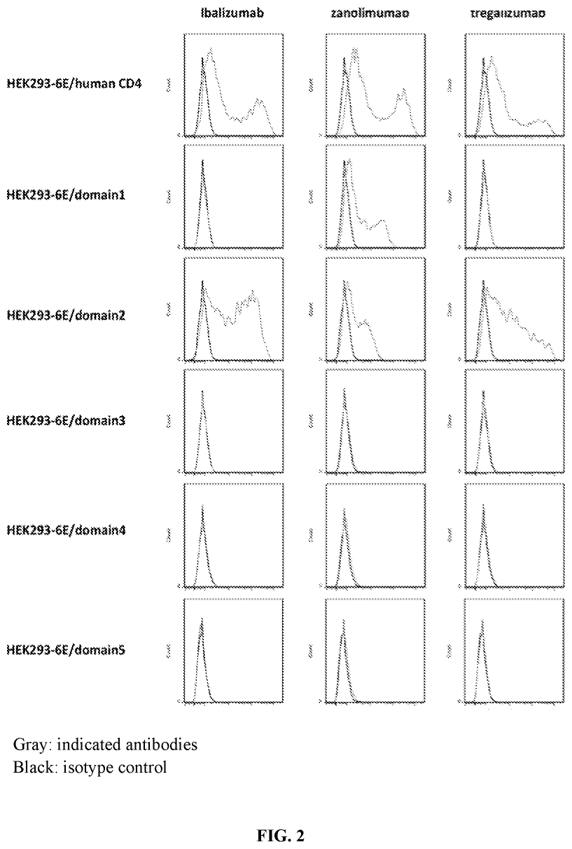

FIG. 1A depicts the structure of an anti-CD4 CAR, which is composed of an CD4 binding moiety (e.g., scFv or sdAb), a hinge region, a transmembrane domain, a co-stimulatory domain and a CD3ζ signaling domain.

SEQ ID NOs of the CAR scFv region of the CAR-T cells used in the example are as follows:

| CAR- | HC- | HC- | HC- | LC- | LC- | LC- | | | |
|---|---|---|---|---|---|---|---|---|---|
| T No. | CDR1 | CDR2 | CDR3 | CDR1 | CDR2 | CDR3 | VH | VL | CAR |
| 1 | 1 | 2 | 3 | 4 | 5 | 6 | 7 | 8 | 33 |
| 4 | 9 | 10 | 11 | 12 | 13 | 14 | 15 | 16 | 34 |
| 5 | 17 | 18 | 19 | 20 | 21 | 22 | 23 | 24 | 35 |
| 2 | 25 | 26 | 27 | 28 | 29 | 30 | 31 | 32 | 36 |
| 3 | 46 | 47 | 48 | 49 | 50 | 51 | 52 | 53 | 54 |
| 6 | 55 | 56 | 57 | 58 | 59 | 60 | 61 | 62 | 63 |

Transmembrane domain (CD8α transmembrane domain): SEQ ID NO: 37
Co-stimulatory domain (4-1-BB co-stimulatory domain): SEQ ID NO: 38
CD3ζ signaling domain: SEQ ID NO: 39
Hinge domain (CD8α hinge domain): SEQ ID NO: 40
CD3ϵ transmembrane domain: SEQ ID NO: 41
CD3ϵ signaling domain: SEQ ID NO: 42
CD3ϵ extracellular domain: SEQ ID NO: 43
Full-length CD3ϵ: SEQ ID NO: 44
Full-length human CD4: SEQ ID NO: 45
Anti-CD4 eTCR: SEQ ID NO: 64

The CAR+ % rate was 13.9% in the CAR-T No. 1 cells, and the CAR+ % rate was 44.2% in No. 2 cells. The CAR+ % were higher in the No. 2 cells than No. 1, but the killing effect was not correlated with the CAR+ percentage. The CD4+% was 0% in No. 1 total cell population, and it was 17.2% in No. 2 total cell population. The CD4+ cells were mostly CAR+ cells, as indicated in the CAR+ population in No. 2 cells in FIG. 1B. The No. 2 CAR+population is thus less susceptible to CAR-T killing. It was reported that anti-CD19 CAR could block the CD19 antigen on the same cells (i.e., in-cis blocking) and leading to the protection of CAR transduced leukemia cells from being killed by CAR-T cells (reference: Nature Medicine volume 24, pages 1499-1503 (2018)). The phenotype of our No. 2 CAR-T suggests that CAR may block the CD4 on the same cell from killing by a second CAR. The protection of self was not observed on No. 1 CAR-T cells.

Since all the CARs were generated in the same way and their only difference is the scFv region. The scFv may cause the different phenotypes we saw between CAR-T No. 1 and No. 2. The scFv in CAR-T No. 1 and No. 2 were derived from Zanolimumab and Ibalizumab respectively. A domain mapping experiment was carried out to detect which CD4 domains these antibodies recognize. One additional antibody, Tregalizumab, was also included in this experiment.

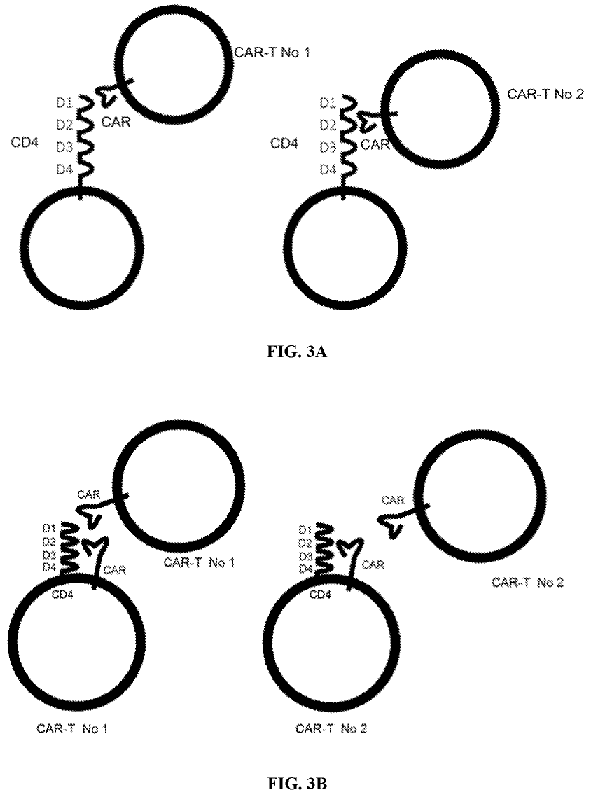

CD4 is a member of immunoglobulin superfamily. It contains four extracellular immunoglobulin domains, Domain 1 to 4 from distal to proximal to cell membrane. The four CD4 extracellular domains and its intracellular domain were named D1-D5 and were expressed transiently with a mouse CD4 backbone in HEK-293 cells. The three antibodies were used to detect human CD4 D1-D5 expression by flow cytometry on these 293 cells. As shown in FIG. 2, Ibalizumab and Tregalizumab interacted with human CD4 domain 2, while Zanolimumab mainly recognized human CD4 domain 1.

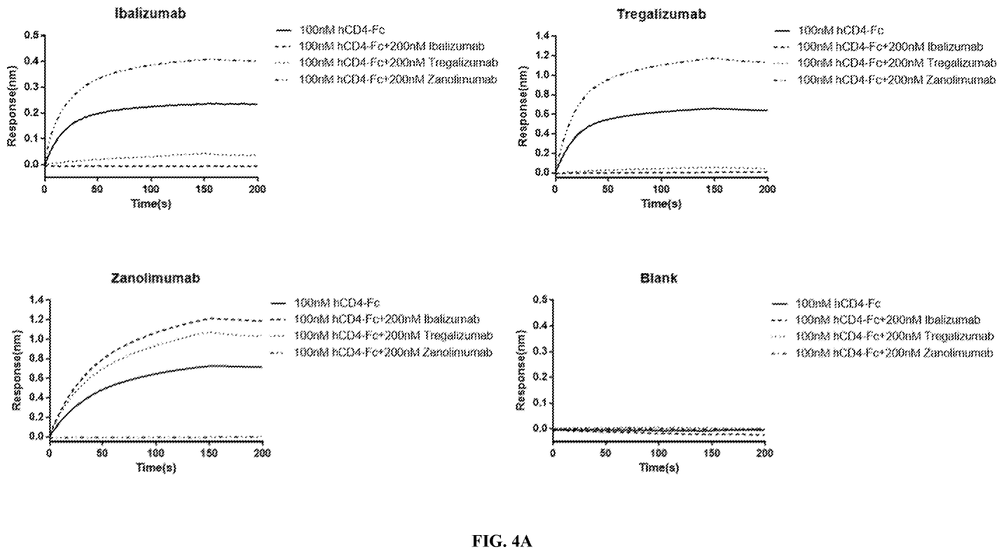

Based on the results discussed herein, an interaction model was hypothesized as illustrated in FIGS. 3A-3B. CAR-T No. 1 bears an scFv that can recognize human CD4 Domain 1, while CAR-T No. 2 has an scFv that can recognize Domain 2 as indicated in FIG. 3A. The proximal domains to the cell membrane is within shorter distance to the chimeric antigen receptors that are expressed on the same cell surface, thus the chimeric antigen receptor may be able to bind to it as showed on the right in FIG. 3B. The interaction between the chimeric antigen receptor and CD4 on the same cell will prevent the CD4 from being recognized by another CAR-T, thus protect the cell from being killed by a second CAR-T cell.

### Example 3. Antibody Blocking Assays

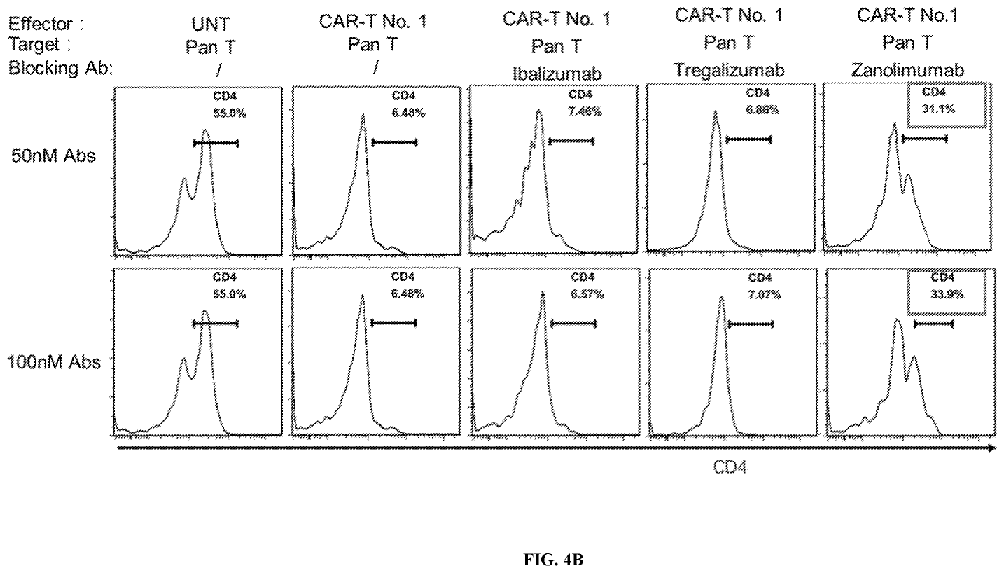

Anti-CD4 antibodies were used to mimic the in-cis interaction between the CAR scFv region and the CD4 molecule. Three antibodies, Ibalizumab, Tregalizumab, Zanolimumab, which mainly recognize CD4 Domain 2, Domain2, and Domain 1 respectively in a flow cytometry assay (FIG. 2), were used in the blocking assay. First, an epitope binning experiment was performed to exam whether the three antibodies compete for the same CD4 binding site. As shown in FIG. 4A, Ibalizumab and Tregalizumab compete with each other for their binding to human CD4 protein. The influence of Ibalizumab or Tregalizumab on Zanolimumab-CD4 interaction was minor. Second, these antibodies were used to test whether they could block the CAR-T mediated target cell killing (FIG. 4B). In the antibody blocking experiment, CAR-T No. 1, which interacts with CD4 Domain 1, was used as effector cells. As shown in FIG. 4B, there were 55% CD4+ cells when the target cells were co-cultured with control UNT cells. The percentage dropped to 6.5% when the target cells were incubated with CAR-T No. 1 effector cells. The percentage of CD4+ cells remained at ˜7% when Ibalizumab or Tregalizumab was added to the culture, suggesting these two antibodies do not block the CAR-T No. 1 mediated target cell recognition and killing. The CD4+ percentage increased to more than 30% when Zanolimumab antibody was added to the culture, suggesting the CAR-T No. 1 mediated killing could be blocked by the domain 1 recognition Zanolimumab antibody. These results indicate that chimeric antigen receptor interaction with CD4 on the same cell could block the recognition of CD4 by another CAR-T cell. The quantitative analysis of FIG. 4B for this experiment was shown in FIG. 4C.

### Example 4. Assays for Anti-CD4 CAR-T Cells

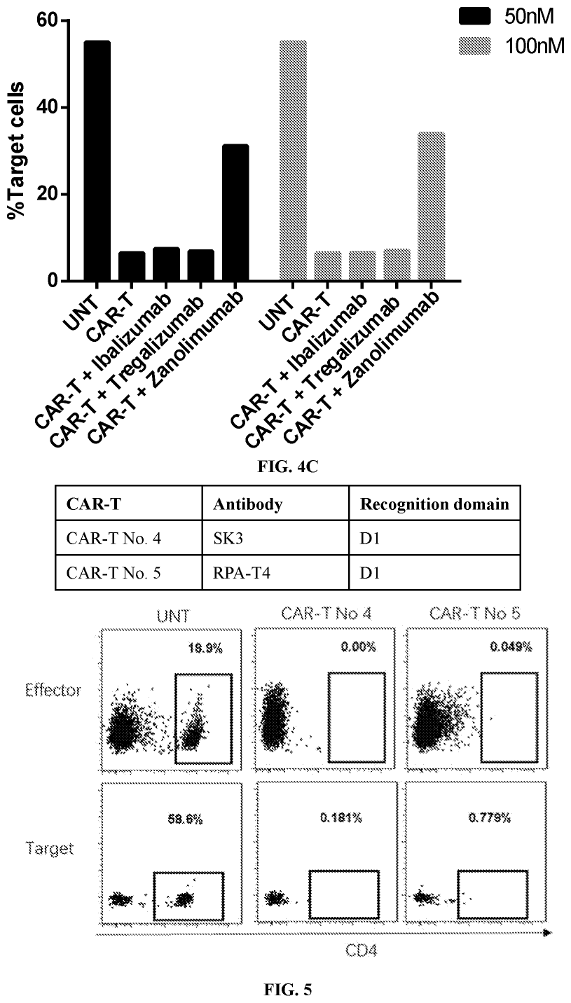

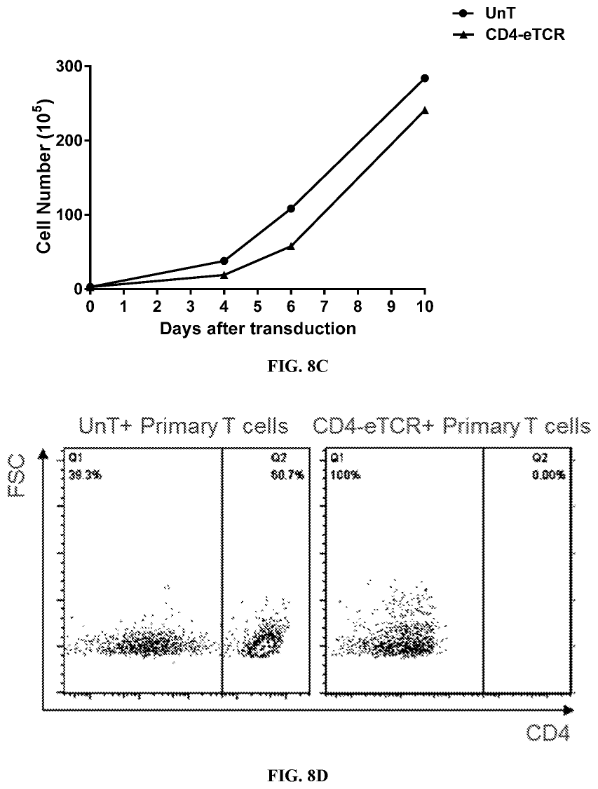

For autologous therapy, when the patient's own T cells were used to generate CAR-T cells, the anti-CD4 CAR-T recognizing CD4 domain 1 is preferred to anti-CD4 CAR-T recognizing other domains. Domain 1 targeting anti-CD4 CAR-T do not block CD4 in-cis and can eliminate CD4+ cells in both the CAR+ and CAR− population to avoid any possible HIV infected CD4+ T cell contamination or malignant T cell contamination in the CAR-T product. To further prove the advantage of anti-CD4 domain 1 CAR-T, two more anti-CD4 CAR-T cells recognizing domain 1 of CD4 were tested. The data is presented in FIG. 5. Both CAR-T No. 4 and No. 5 recognize CD4 Domain 1. The scFv in CAR-T No. 4 and No. 5 were derived from SK3 and RPA-T4 respectively. To prove the self-protection effects for antibodies recognizing other domains of CD4, two more anti-CD4 CAR-T (domain 2-3) were tested. The data is presented in FIG. 9. Both CAR-T No. 3 and No. 6 cells recognize CD4 Domain 2-3.

Un-transduced pan T cells (UNT) were used as negative control. UNT and CAR-T cells were co-cultured with CFSE labeled pan T cells for 24 hours before they were harvested for flow cytometry. Effector cell population and target cell population were distinguished by CFSE. In the control UNT samples, 18.9% of effector cells were CD4+ after co-culture. There were 0% of CD4+ cells in the effector population of No. 4 cells. For CAR-T No. 5, the CD4+ percentage in both effector and target population were less than 1%. In contrast, there were 12.5% and 13.1% of CD4+ cells in the effector population of No. 3 and No. 6 cells. This further indicates the anti-CD4 domain 1 CAR-T can eliminate CD4+population in both the CAR-T cells and the target cells, that there is no in-cis blocking in the CAR-T cells.

### Example 5. Cell Killing Assays of Anti-CD4 CAR-T Cells

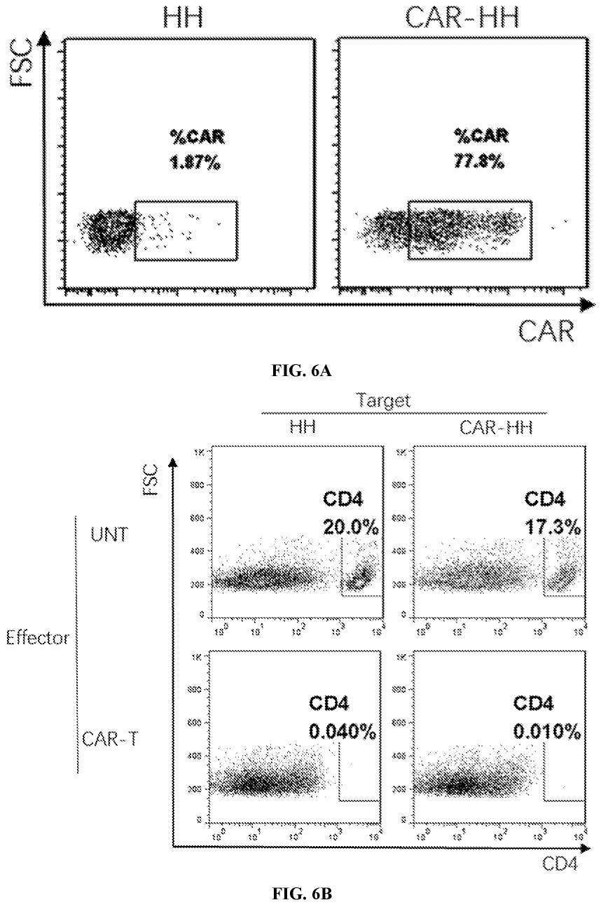

To further demonstrate that anti-CD4 CAR-T cells do not have in-cis protection for the CD4 molecule expressed on the same cells as the CAR, a CD4+ T lymphoma cell line HH was transduced with the CAR lentiviruses. The data are presented in FIG. 6A shows that 77.8% of HH cells were CAR+ after transduction. These cells express both CD4 and anti-CD4 Domain 1 CAR and were named as CAR-HH cells. CAR-HH cells and HH cells alone were co-cultured with anti-CD4 domain 1 CAR-T No. 1 cells or control UNT cells. FIGS. 6B-6C show that after 8 days of culture, there were 20% of CD4+ cells in the UNT treated HH cells, and 17.3% of CD4+ cells in the UNT treated CAR-HH cells. However, the percentage of remaining CD4+ cells were less than 0.1% in both the HH and CAR-HH sample co-cultured with CAR-T cells. The CAR-T cells could kill the HH cells no matter whether they express a CAR or not. These data proved that the anti-CD4 domain 1 CAR do not have the in-cis block the CD4 antigen been recognized by an anti-CD4 domain CAR-T. They can eliminate residue virus infected CD4 T cells or CD4 T lymphoma cells contaminated in the CAR-T products if autologous therapy is desired.

### Example 6. In Vivo Analysis of Anti-CD4 CAR-T Cells

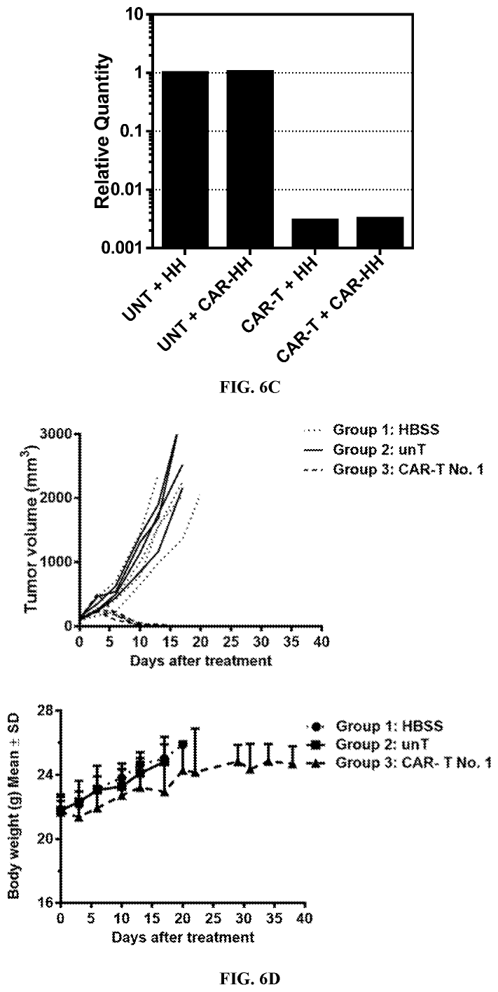

To test whether the anti-CD4 domain 1 CAR-T cells are effective in vivo, mice with human immune system and rhesus experiment were utilized. The adult HIS mice with human T cells were intravenously injected with anti-CD4 CAR-T cells or UNT cells. The CD4/CD8 ratio in the mice spleen at day 18 post treatment is shown in FIG. 7. The CD4+ percentage was 43.1% in the UNT mouse spleen, while the percentage dropped to 1.25% in the CAR-T mouse spleen. These data suggest that the anti-CD4 domain 1 CAR-T No. 1 cells were very effective in eliminating CD4+ cells in vivo.

The efficacy of anti-CD4 domain 1 CAR-T cells were also assessed in cell-derived xenograft mouse (CDX) models. Mice transplanted with HH T cell lymphoma cells were treated with the anti-CD4 CAR-T No. 1 cells, HBSS buffer, or UNT cells. As shown in FIG. 6D, the tumor size was reduced to 0 within 15 days post CAR-T treatment, while in the two control groups, the tumor grew continuously until the end of the experiment or until the mice had to be sacrificed due to the tumor burden.

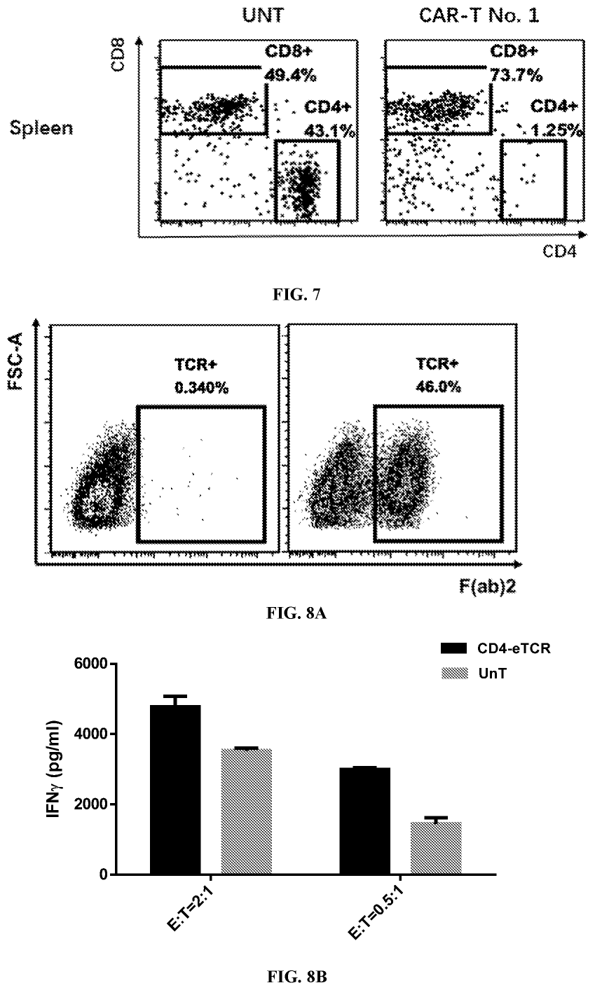

The anti-CD4 domain 1 scFvs were also constructed into a chimer T cell receptor (“cTCR”). In this example, it was linked to CD3ε, thus was named as anti-CD4 eTCR. As shown in FIG. 8A, 46% of T cells were eTCR+ after transduction. The anti-CD4 eTCR cells produced IFNγ when cultured with pan T cell target cells, but the level was only increased slightly. FIG. 8C shows the expansion of anti-CD4 eTCR cells. The cells expanded vigorously within 10 days in culture. FIG. 8D shows the target cell killing by these anti-CD4 eTCR cells. The CFSE labeled pan T cells were used as target cells and was co-cultured with the anti-CD4 eTCR cells for 24 hours before they were harvested for flow cytometry. The anti-CD4 eTCR cells could eliminate all the CD4+ T cells as shown on the right of FIG. 8D.

### Example 7. Assays for Anti-CD22 CAR-T

Similar to the example of CD4, we hypothesized that there can be a self-protective effect when there are 3 domains near the cell membrane. The proximal domains to the cell membrane is within shorter distance (within 3 domains) to the chimeric antigen receptors that are expressed on the same cell surface, thus the chimeric antigen receptor may be able to bind to it. The interaction between the chimeric antigen receptor and CD22 on the same cell will prevent the CD22 from being recognized by another anti-CD22 CAR-T, thus protect the cell from being killed by a second anti-CD22 CAR-T cell.

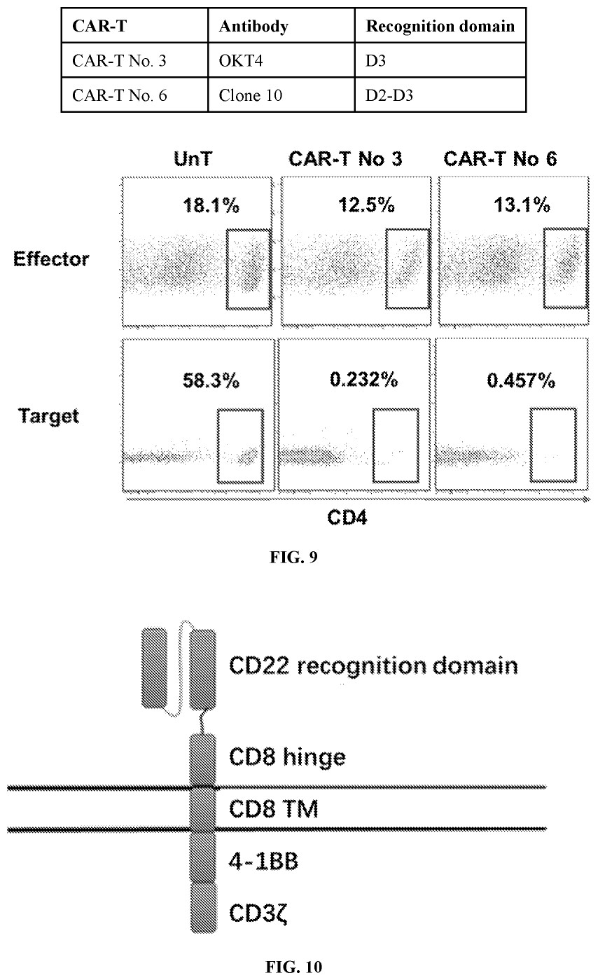

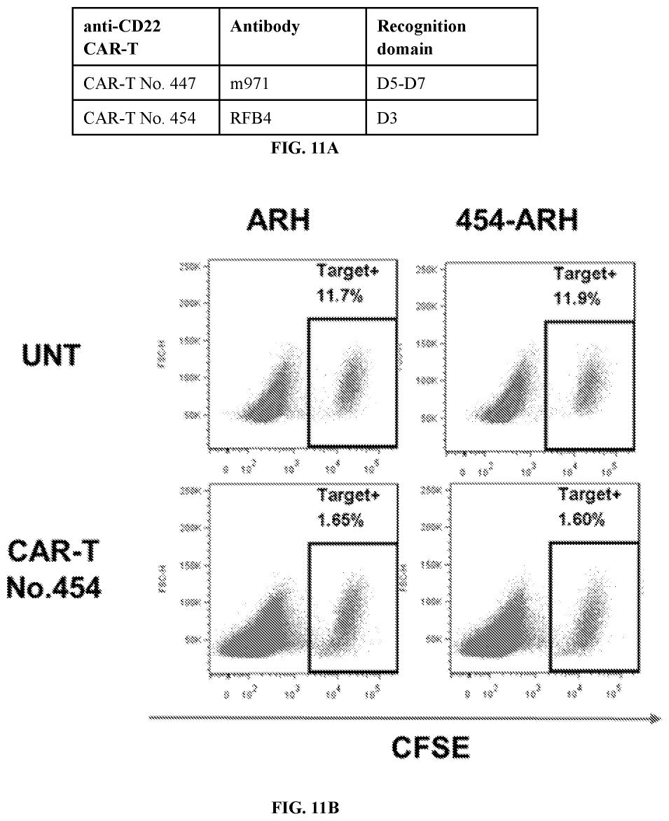

To test this hypothesis, two anti-CD22 CAR-Ts were tested (FIGS. 10 and 11A). CAR-T No. 454 recognizes Domain 3 of CD22, and CAR-T No. 447 recognizes domain 5-7 (3 domains near the cell membrane) of CD22. UNT cells (un-transduced T cells) and CAR-T cells were co-cultured with CFSE-labeled pan T target cells (“ARH cells”) at E:T (effector:target) ratio of 0.5:1 for 24 hours. The remaining target cells were detected by flow cytometry.

The sequence of the CAR scFv region of the anti-CD22 CAR-T cells are as follows:

| CAR- | HC- | HC- | HC- | LC- | LC- | LC- | | | |
|---|---|---|---|---|---|---|---|---|---|
| T No. | CDR1 | CDR2 | CDR3 | CDR1 | CDR2 | CDR3 | VH | VL | CAR |
| 454 | 67 | 68 | 69 | 70 | 71 | 72 | 73 | 74 | 75 |
| 447 | 76 | 77 | 78 | 79 | 80 | 81 | 82 | 83 | 84 |

Transmembrane domain (CD8α transmembrane domain): SEQ ID NO: 37
Co-stimulatory domain (4-1-BB co-stimulatory domain): SEQ ID NO: 38
CD3ζ signaling domain: SEQ ID NO: 39
Hinge domain (CD8α hinge domain): SEQ ID NO: 40
CD3ϵ transmembrane domain: SEQ ID NO: 41
CD3ϵ signaling domain: SEQ ID NO: 42
CD3ϵ extracellular domain: SEQ ID NO: 43
Full-length CD3ϵ: SEQ ID NO: 44
Full-length human CD22: SEQ ID NO: 66

As shown in FIGS. 11B and 11C, target ARH and CAR454-ARH cells could be killed by CAR-T No. 454, demonstrating that CAR-T No. 454 has no protection for itself. By contrast, CAR-T No. 447, which recognizes Domains 5-7, was only able to kill the target ARH cells, with 8.15% of CAR447-ARH cells remaining. This demonstrates that CAR-T No. 447 has protective effects on cells that co-express the CAR and CD22.

### SEQUENCE LISTING

Sequences of exemplary constructs according to embodiments of the invention:

| | Seq | CDR1 | Seq | CDR2 | Seq | CDR3 |
|---|---|---|---|---|---|---|
| Name | ID | Sequence | ID | Sequence | ID | Sequence |
| CAR-T No. 1 | 1 | GGSFSGY | 2 | NHSGS | 3 | VINWFDP |
| VH | | | | | | |
| CAR-T No. 1 | 4 | RASQDISSW | 5 | AASSLQS | 6 | QQANSFPYT |
| VL | | LA | | | | |
| CAR-T No. 4 | 9 | GYTFTDYV | 10 | TYTGSGSS | 11 | RGKGTGFAF |
| VH | | | | | | |
| CAR-T No.4 | 12 | QSVDYDGDS | 13 | AASNLES | 14 | QQSYEDPPT |
| VL | | Y | | | | |
| CAR-T No. 5 | 17 | GYTFTNY | 18 | DPSTGY | 19 | EGGIGGFAY |
| VH | | | | | | |
| CAR-T No. 5 | 20 | RASESVDSY | 21 | RASNLES | 22 | QQSKEDPYT |
| VL | | DNSFMH | | | | |
| CAR-T No. 2 | 25 | GYTFTSY | 26 | NPYNDG | 27 | EKDNYATGAWFAY |
| VH | | | | | | |
| CAR-T No. 2 | 28 | KSSQSLLYS | 29 | WASTRES | 30 | QQYYSYRT |
| VL | | TNQK | | | | |
| CAR-T No. 3 | 46 | GFSFSDC | 47 | SVKSENYG | 48 | SYYRYDVGAWFAY |
| VH | | | | | | |
| CAR-T No. 3 | 49 | RASKSVSTS | 50 | LASILES | 51 | QHSRELPWT |
| VL | | GYSYIY | | | | |
| CAR-T No. 6 | 55 | GYTFTNY | 56 | NTNTGE | 57 | LGLYYDYGYYAM |
| VH | | | | | | |
| CAR-T No. 6 | 58 | RASESVDSY | 59 | LASNLES | 60 | QQNNEDPYT |
| VL | | GN | | | | |
| CAR-T No. | 67 | GFAFSIYDM | 68 | YISSGGGTTYYP | 69 | HSGYGSSYGVLFAY |
| 545 VH | | S | | DTVKG | | |
| CAR-T No. | 70 | RASQDISNY | 71 | YTSILHS | 72 | QQGNTLPWT |
| 545 VL | | LN | | | | |
| CAR-T No. | 76 | GDSVSSNSA | 77 | RTYYRSKWYND | 78 | EVTGDLEDAFDI |
| 447 VH | | AWN | | YAVSVKS | | |
| CAR-T No. | 79 | RASQTIWSY | 80 | AASSLQS | 81 | QQSYSIPQT |
| 447 VL | | LN | | | | |

SEQ ID NO 07: (CAR No. 1 VH amino acid sequence)
QVQLQQWGAGLLKPSETLSLTCAVYGGSFSGYYWSWIRQPPGKGLEWIGEINHSGSTN
YNPSLKSRVTISVDTSKNQFSLKLSSVTAADTAVYYCARVINWFDPWGQGTLVT
SEQ ID NO 08: (CAR No. 1 VL amino acid sequence)
DIQMTQSPSSVSASVGDRVTITCRASQDISSWLAWYQHKPGKAPKLLIYAASSLQSGVPS
RFSGSGSGTDFTLTISSLQPEDFATYYCQQANSFPYTFGQGTKLEIK
SEQ ID NO 15: (CAR-T No. 4 VH amino acid sequence)
QVQLQQSGPELVKPGASVKMSCKASGYTFTDYVINWVKQRTGQGLEWIGETYTGSGSS
YYNEKFKDKATLTVDKASNIAYMQLSSLTSEDSAVYFCARRGKGTGFAFWGQGTLVT
VSA
SEQ ID NO 16: (CAR-T No. 4 VL amino acid sequence)
DIVLTQSPASLAVSLGQRATISCKASQSVDYDGDSYMNWYQQKPGQPPKLLIYAASNLE
SGIPARFTGSGSGTDFTLNIHPVEEEDTATYYCQQSYEDPPTFAGGTNLEIK
SEQ ID NO 23: (CAR-T No. 5 VH amino acid sequence)
QVQLQQSGAELAKPGASVKMSCKASGYTFTNYLMHWVKQRPGQGLEWIGYIDPSTGY
TVYLQKFKDKATLTADKSSSTTYMQLSSLTSEDSAVYYCAKEGGIGGFAYWGQGTLVT
VSA
SEQ ID NO 24: (CAR-T No. 5 VL amino acid sequence)
DIVLTPSPASLAVSLGQRATISCRASESVDSYDNSFMHWYQQKPGQPPKWYRASNLES
GIPARFSGSGSRTDFTLTIDPVEADDVATYYCQQSKEDPYTFGGGTKLEIK
SEQ ID NO 31: (CAR-T No. 2 VH amino acid sequence)
QVQLQQSGPEVVKPGASVKMSCKASGYTFTSYVIHWVRQKPGQGLDWIGYINPYNDG
TDYDEKFKGKATLTSDTSTSTAYMELSSLRSEDTAVYYCAREKDNYATGAWFAYWGQ
GTLVTVSSA
SEQ ID NO 32: (CAR-T No. 2 VL amino acid sequence)
DIVMTQSPDSLAVSLGERVTMNCKSSQSLLYSTNQKNYLAWYQQKPGQSPKLLIYWAS
TRESGVPDRFSGSGSGTDFTLTISSVQAEDVAVYYCQQYYSYRTFGGGTKLEIKR
SEQ ID NO 33: (CAR No. 1 amino acid sequence)
MALPVTALLLPLALLLHAARPQVQLQQWGAGLLKPSETLSLTCAVYGGSFSGYYWSWI
RQPPGKGLEWIGEINHSGSTNYNPSLKSRVTISVDTSKNQFSLKLSSVTAADTAVYYCAR
VINWFDPWGQGTLVTGGGGSGGGGSGGGGSDIQMTQSPSSVSASVGDRVTITCRASQDI
SSWLAWYQHKPGKAPKLLIYAASSLQSGVPSRFSGSGSGTDFTLTISSLQPEDFATYYCQ
QANSFPYTFGQGTKLEIKTTTPAPRPPTPAPTIASQPLSLRPEACRPAAGGAVHTRGLDFA
CDIYIWAPLAGTCGVLLLSLVITLYCKRGRKKLLYIFKQPFMRPVQTTQEEDGCSCRFPE
EEEGGCELRVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATKDTYDALH
MQALPPR
SEQ ID NO 34: (CAR No. 4 amino acid sequence)
MALPVTALLLPLALLLHAARPQVQLQQSGPELVKPGASVKMSCKASGYTFTDYVINWV
KQRTGQGLEWIGETYTGSGSSYYNEKFKDKATLTVDKASNIAYMQLSSLTSEDSAVYF
CARRGKGTGFAFWGQGTLVTVSAGGGGSGGGGSGGGGSDIVLTQSPASLAVSLGQRAT
ISCKASQSVDYDGDSYMNWYQQKPGQPPKLLIYAASNLESGIPARFTGSGSGTDFTLNIH
PVEEEDTATYYCQQSYEDPPTFAGGTNLEIKTTTPAPRPPTPAPTIASQPLSLRPEACRPA
AGGAVHTRGLDFACDIYIWAPLAGTCGVLLLSLVITLYCKRGRKKLLYIFKQPFMRPVQ
TTQEEDGCSCRFPEEEEGGCELRVKFSRSADAPAYKQGQNQLYNELNLGRREEYDVLD
KRRGRDPEMGGKPRRKNPQEGLYNELQKDKMAEAYSEIGMKGERRRGKGHDGLYQG
LSTATKDTYDALHMQALPPR
SEQ ID NO 35: (CAR No. 5 amino acid sequence)
MALPVTALLLPLALLLHAARPQVQLQQSGAELAKPGASVKMSCKASGYTFTNYLMEIW
VKQRPGQGLEWIGYIDPSTGYTVYLQKFKDKATLTADKSSSTTYMQLSSLTSEDSAVYY
CAKEGGIGGFAYWGQGTLVTVSAGGGGSGGGGSGGGGSDIVLTPSPASLAVSLGQRAT
ISCRASESVDSYDNSFMHWYQQKPGQPPKLLIYRASNLESGIPARFSGSGSRTDFTLTIDP
VEADDVATYYCQQSKEDPYTFGGGTKLEIKTTTPAPRPPTPAPTIASQPLSLRPEACRPA
AGGAVHTRGLDFACDIYIWAPLAGTCGVLLLSLVITLYCKRGRKKLLYIFKQPFMRPVQ
TTQEEDGCSCRFPEEEEGGCELRVKFSRSADAPAYKQGQNQLYNELNLGRREEYDVLD
KRRGRDPEMGGKPRRKNPQEGLYNELQKDKMAEAYSEIGMKGERRRGKGHDGLYQG
LSTATKDTYDALHMQALPPR
SEQ ID NO 36: (CAR No. 2 amino acid sequence)
MALPVTALLLPLALLLHAARPQVQLQQSGPEVVKPGASVKMSCKASGYTFTSYVIHWV
RQKPGQGLDWIGYINPYNDGTDYDEKFKGKATLTSDTSTSTAYMELSSLRSEDTAVYY
CAREKDNYATGAWFAYWGQGTLVTVSSAGGGGSGGGGSGGGGSDIVMTQSPDSLAVS
LGERVTMNCKSSQSLLYSTNQKNYLAWYQQKPGQSPKLLIYWASTRESGVPDRFSGSG
SGTDFTLTISSVQAEDVAVYYCQQYYSYRTFGGGTKLEIKRTTTPAPRPPTPAPTIASQPL
SLRPEACRPAAGGAVHTRGLDFACDIYIWAPLAGTCGVLLLSLVITLYCKRGRKKLLYIF
KQPFMRPVQTTQEEDGCSCRFPEEEEGGCELRVKFSRSADAPAYKQGQNQLYNELNLG
RREEYDVLDKRRGRDPEMGGKPRRKNPQEGLYNELQKDKMAEAYSEIGMKGERRRGK
GHDGLYQGLSTATKDTYDALHMQALPPR
SEQ ID NO 37: (CD8α transmembrane domain amino acid sequence)
IYIWAPLAGTCGVLLLSLVITLYC
SEQ ID NO 38: (4-1BB co-stimulatory domain amino acid sequence)
KRGRKKLLYIFKQPFMRPVQTTQEEDGCSCRFPEEEEGGCEL
SEQ ID NO 39: (CD3ζ signaling domain amino acid sequence)
RVKFSRSADAPAYKQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
SEQ ID NO 40: (CD8α hinge domain amino acid sequence)
TTTPAPRPPTPAPTIASQPLSLRPEACRPAAGGAVHTRGLDFACD
SEQ ID NO 41: (CD3ϵ transmembrane domain amino acid sequence)
VMSVATIVIVDICITGGLLLLVYYWS
SEQ ID NO 42: (CD3ϵ signaling domain amino acid sequence)
MQSGTHWRVLGLCLLSVGVWGQ
SEQ ID NO 43: (CD3ϵ extracellular domain amino acid sequence)
DGNEEMGGITQTPYKVSISGTTVILTCPQYPGSEILWQHNDKNIGGDEDDKNIGSDEDHL
SLKEFSELEQSGYYVCYPRGSKPEDANFYLYLRARVCENCMEMD
SEQ ID NO 44: (full length CD3ϵ amino acid sequence)
MQSGTHWRVLGLCLLSVGVWGQDGNEEMGGITQTPYKVSISGTTVILTCPQYPGSEIL
WQHNDKNIGGDEDDKNIGSDEDHLSLKEFSELEQSGYYVCYPRGSKPEDANFYLYLRA
RVCENCMEMDVMSVATIVIVDICITGGLLLLVYYWSKNRKAKAKPVTRGAGAGGRQR
GQNKERPPPVPNPDYEPIRKGQRDLYSGLNQRRI
SEQ ID NO 45: (full length human CD4 amino acid sequence)
MNRGVPFRHLLLVLQLALLPAATQGKKVVLGKKGDTVELTCTASQKKSIQFHWKNSN
QIKILGNQGSFLTKGPSKLNDRADSRRSLWDQGNFPLIIKNLKIEDSDTYICEVEDQKEEV
QLLVFGLTANSDTHLLQGQSLTLTLESPPGSSPSVQCRSPRGKNIQGGKTLSVSQLELQD
SGTWTCTVLQNQKKVEFKIDIVVLAFQKASSIVYKKEGEQVEFSFPLAFTVEKLTGSGEL
WWQAERASSSKSWITFDLKNKEVSVKRVTQDPKLQMGKKLPLHLTLPQALPQYAGSG
NLTLALEAKTGKLHQEVNLVVMRATQLQKNLTCEVWGPTSPKLMLSLKLENKEAKVS
KREKAVWVLNPEAGMWQCLLSDSGQVLLESNIKVLPTWSTPVQPMALIVLGGVAGLL
LFIGLGIFFCVRCRHRRRQAERMSQIKRLLSEKKTCQCPHRFQKTCSPI
SEQ ID NO 52: (CAR No. 3 VH amino acid sequence)
EEQLVESGGGLVKPGGSLRLSCAASGFSFSDCRMYWLRQAPGKGLEWIGVISVKSENY
GANYAESVRGRFTISRDDSKNTVYLQMNSLKTEDTAVYYCSASYYRYDVGAWFAYW
GQGTLVTVSSA
SEQ ID NO 53: (CAR No. 3 VL amino acid sequence)
DIVMTQSPDSLAVSLGERATINCRASKSVSTSGYSYIYWYQQKPGQPPKLLIYLASILESG
VPDRFSGSGSGTDFTLTISSLQAEDVAVYYCQHSRELPWTFGQGTKVEIKR
SEQ ID NO 54: (CAR No. 3 amino acid sequence)
MALPVTALLLPLALLLHAARPEEQLVESGGGLVKPGGSLRLSCAASGFSFSDCRMYWL
RQAPGKGLEWIGVISVKSENYGANYAESVRGRFTISRDDSKNTVYLQMNSLKTEDTAV
YYCSASYYRYDVGAWFAYWGQGTLVTVSSAGGGGSGGGGSGGGGSDIVIVITQSPDSL
AVSLGERATINCRASKSVSTSGYSYIYWYQQKPGQPPKLLIYLASILESGVPDRFSGSGSG
TDFTLTISSLQAEDVAVYYCQHSRELPWTFGQGTKVEIKRTTTPAPRPPTPAPTIASQPLS
LRPEACRPAAGGAVHTRGLDFACDIYIWAPLAGTCGVLLLSLVITLYCKRGRKKLLYIF
KQPFMRPVQTTQEEDGCSCRFPEEEEGGCELRVKFSRSADAPAYKQGQNQLYNELNLG
RREEYDVLDKRRGRDPEMGGKPRRKNPQEGLYNELQKDKMAEAYSEIGMKGERRRGK
GHDGLYQGLSTATKDTYDALHMQALPPR
SEQ ID NO 61: (CAR No. 6 VH amino acid sequence)
QIQLVQSGPELKKPGETVKISCKASGYTFTNYGMNWVKQAPGKGLKCMGWINTNTGEP
TYAEEFKGRFAFSLETSATTAFLQINNLKDEDTATYFCARLGLYYDYGYYAMDYWGQ
GASVTVSS
SEQ ID NO 62: (CAR No. 6 VL amino acid sequence)
NIVLTQSPASLAVSLGQRATISCRASESVDSYGNSFMEIWYQQKPGQPPKLFIYLASNLES
GVPARFSGSGSRTDFTLTIDPVEADDAATYYCQQNNEDPYTFGGGTKLEIK
SEQ ID NO 63: (CAR No. 6 amino acid sequence)
MALPVTALLLPLALLLHAARPQIQLVQSGPELKKPGETVKISCKASGYTFTNYGMNWV
KQAPGKGLKCMGWINTNTGEPTYAEEFKGRFAFSLETSATTAFLQINNLKDEDTATYFC
ARLGLYYDYGYYAMDYWGQGASVTVSSGGGGSGGGGSGGGGSNIVLTQSPASLAVSL
GQRATISCRASESVDSYGNSFMHWYQQKPGQPPKLFIYLASNLESGVPARFSGSGSRTD
FTLTIDPVEADDAATYYCQQNNEDPYTFGGGTKLEIKTTTPAPRPPTPAPTIASQPLSLRP
EACRPAAGGAVHTRGLDFACDIYIWAPLAGTCGVLLLSLVITLYCKRGRKKLLYIFKQP
FMRPVQTTQEEDGCSCRFPEEEEGGCELRVKFSRSADAPAYKQGQNQLYNELNLGRRE
EYDVLDKRRGRDPEMGGKPRRKNPQEGLYNELQKDKMAEAYSEIGMKGERRRGKGH
DGLYQGLSTATKDTYDALHMQALPPR
SEQ ID NO: 64: (Anti-CD4 eTCR)
MQSGTHWRVLGLCLLSVGVWGQQVQLQQWGAGLLKPSETLSLTCAVYGGSFSGYYW
SWIRQPPGKGLEWIGEINHSGSTNYNPSLKSRVTISVDTSKNQFSLKLSSVTAADTAVYY
CARVINWFDPWGQGTLVTGGGGSGGGGSGGGGSDIQMTQSPSSVSASVGDRVTITCRA
SQDISSWLAWYQHKPGKAPKLLIYAASSLQSGVPSRFSGSGSGTDFTLTISSLQPEDFAT
YYCQQANSFPYTFGQGTKLEIKGGGGSGGGGSGGGGSDGNEEMGGITQTPYKVSISGTT
VILTCPQYPGSEILWQHNDKNIGGDEDDKNIGSDEDHLSLKEFSELEQSGYYVCYPRGSK
PEDANFYLYLRARVCENCMEMDVMSVATIVIVDICITGGLLLLVYYWSKNRKAKAKPV
TRGAGAGGRQRGQNKERPPPVPNPDYEPIRKGQRDLYSGLNQRRI
SEQ ID NO: 65
CC(A/T)₆GG
SEQ ID NO 66: (full length human CD22 amino acid sequence)
MHLLGPWLLLLVLEYLAFSDSSKWVFEHPETLYAWEGACVWIPCTYRALDGDLESFILF
HNPEYNKNTSKFDGTRLYESTKDGKVPSEQKRVQFLGDKNKNCTLSIHPVHLNDSGQL
GLRMESKTEKWMERIHLNVSERPFPPHIQLPPEIQESQEVTLTCLLNFSCYGYPIQLQWL
LEGVPMRQAAVTSTSLTIKSVFTRSELKFSPQWSHEIGKIVTCQLQDADGKFLSNDTVQL
NVKHTPKLEIKVTPSDAIVREGDSVTMTCEVSSSNPEYTTVSWLKDGTSLKKQNTFTLN
LREVTKDQSGKYCCQVSNDVGPGRSEEVFLQVQYAPEPSTVQILHSPAVEGSQVEFLCM
SLANPLPTNYTWYHNGKEMQGRTEEKVHIPKILPWHAGTYSCVAENILGTGQRGPGAE
LDVQYPPKKVTTVIQNPMPIREGDTVTLSCNYNSSNPSVTRYEWKPHGAWEEPSLGVLK
IQNVGWDNTTIACAACNSWCSWASPVALNVQYAPRDVRVRKIKPLSEIHSGNSVSLQC
DFSSSHPKEVQFFWEKNGRLLGKESQLNFDSISPEDAGSYSCWVNNSIGQTASKAWTLE
VLYAPRRLRVSMSPGDQVMEGKSATLTCESDANPPVSHYTWFDWNNQSLPYHSQKLR
LEPVKVQHSGAYWCQGTNSVGKGRSPLSTLTVYYSPETIGRRVAVGLGSCLAILILAICG
LKLQRRWKRTQSQQGLQENSSGQSFFVRNKKVRRAPLSEGPHSLGCYNPMMEDGISYT
TLRFPEMNIPRTGDAESSEMQRPPPDCDDTVTYSALHKRQVGDYENVIPDFPEDEGIHYS
ELIQFGVGERPQAQENVDYVILKH
SEQ ID NO 73: (CAR-T No. 454 VH amino acid sequence)
EVQLVESGGGLVKPGGSLKLSCAASGFAFSIYDMSWVRQTPEKRLEWVAYISSGGGTT
YYPDTVKGRFTISRDNAKNTLYLQMSSLKSEDTAMYYCARHSGYGSSYGVLFAYWGQ
GTLVTVSA
SEQ ID NO 74: (CAR-T No. 454 VL amino acid sequence)
DIQMTQTTSSLSASLGDRVTISCRASQDISNYLNWYQQKPDGTVKLLIYYTSILHSGVPS
RFSGSGSGTDYSLTISNLEQEDFATYFCQQGNTLPWTFGGGTKLEIK
SEQ ID NO 75: (CAR No. 454 amino acid sequence)
MALPVTALLLPLALLLHAARPEVQLVESGGGLVKPGGSLKLSCAASGFAFSIYDMSWV
RQTPEKRLEWVAYISSGGGTTYYPDTVKGRFTISRDNAKNTLYLQMSSLKSEDTAMYY
CARHSGYGSSYGVLFAYWGQGTLVTVSAGGGGSGGGGSGGGGSDIQMTQTTSSLSASL
GDRVTISCRASQDISNYLNWYQQKPDGTVKLLIYYTSILHSGVPSRFSGSGSGTDYSLTIS
NLEQEDFATYFCQQGNTLPWTFGGGTKLEIKTTTPAPRPPTPAPTIASQPLSLRPEACRPA
AGGAVHTRGLDFACDIYIWAPLAGTCGVLLLSLVITLYCKRGRKKLLYIFKQPFMRPVQ
TTQEEDGCSCRFPEEEEGGCELRVKFSRSADAPAYKQGQNQLYNELNLGRREEYDVLD
KRRGRDPEMGGKPRRKNPQEGLYNELQKDKMAEAYSEIGMKGERRRGKGHDGLYQG
LSTATKDTYDALHMQALPPR
SEQ ID NO 82: (CAR No. 447 VH amino acid sequence)
QVQLQQSGPGLVKPSQTLSLTCAISGDSVSSNSAAWNWIRQSPSRGLEWLGRTYYRSK
WYNDYAVSVKSRITINPDTSKNQFSLQLNSVTPEDTAVYYCAREVTGDLEDAFDIWGQ
GTMVTVSS
SEQ ID NO 83: (CAR No. 447 VL amino acid sequence)
DIQMTQSPSSLSASVGDRVTITCRASQTIWSYLNWYQQRPGKAPNLLIYAASSLQSGVPS
RFSGRGSGTDFTLTISSLQAEDFATYYCQQSYSIPQTFGQGTKLEIK
SEQ ID NO 84: (CAR No. 447 amino acid sequence)
MALPVTALLLPLALLLHAARPQVQLQQSGPGLVKPSQTLSLTCAISGDSVSSNSAAWN
WIRQSPSRGLEWLGRTYYRSKWYNDYAVSVKSRITINPDTSKNQFSLQLNSVTPEDTAV
YYCAREVTGDLEDAFDIWGQGTMVTVSSGGGGSGGGGSGGGGSDIQMTQSPSSLSASV
GDRVTITCRASQTIWSYLNWYQQRPGKAPNLLIYAASSLQSGVPSRFSGRGSGTDFTLTI
SSLQAEDFATYYCQQSYSIPQTFGQGTKLEIKTTTPAPRPPTPAPTIASQPLSLRPEACRPA
AGGAVHTRGLDFACDIYIWAPLAGTCGVLLLSLVITLYCKRGRKKLLYIFKQPFMRPVQ
TTQEEDGCSCRFPEEEEGGCELRVKFSRSADAPAYKQGQNQLYNELNLGRREEYDVLD
KRRGRDPEMGGKPRRKNPQEGLYNELQKDKMAEAYSEIGMKGERRRGKGHDGLYQG
LSTATKDTYDALHMQALPPR

What is claimed is:

1. An engineered immune cell comprising on its surface a recognition molecule that comprises a binding moiety specifically binding to a target molecule on the surface of a target cell,
wherein the target molecule comprises an extracellular domain,
wherein the binding moiety specifically binds to a distal portion of the extracellular domain,
wherein the immune cell is capable of killing a target cell that comprises on its surface the target molecule, and
wherein the immune cell is capable of killing a target cell that comprises on its surface both the target molecule and the recognition molecule.

2. The engineered immune cell of claim 1, wherein:
(i) the distance from the distal portion of the extracellular domain to the membrane of the target cell is more than about 0.5 times of the distance from the binding moiety to the membrane of engineered immune cell;
(ii) the extracellular domain of the target molecule is at least about 175 amino acids long, optionally wherein the binding moiety binds to a region in the extracellular domain that is about 50 amino acids or more away from the C-terminus of the extracellular domain; and/or optionally wherein the binding moiety binds to a region that is within about 80 amino acids from the N-terminus of the extracellular domain; and/or
(iii) the distal portion of the extracellular domain is at least about 30 Å away from the membrane of the target cell.

3. The engineered immune cell of claim 1 or 2, wherein the extracellular domain of the target molecule comprises three or more Ig-like domains, and wherein:
(i) the binding moiety binds to a region outside the first two Ig-like domains from the C-terminal end of the extracellular domain; and/or
(ii) the binding moiety binds to a region within the first Ig-like domain at the N-terminal end of the extracellular domain.

4. The engineered immune cell of any one of claims 1-3, wherein:
(i) the binding moiety competes for binding with a reference antibody that specifically binds to an epitope within Domains 1-4 of CD22 (“anti-CD22 D1-4 antibody”);
(ii) the binding moiety binds to an epitope in Domains 1-4 of CD22 that overlaps with the binding epitope of a reference anti-CD22 D1-4 antibody;
(iii) the binding moiety comprises the same heavy chain and light chain CDR sequences as those of a reference anti-CD22 D1-4 antibody; and/or
(iv) the binding moiety comprises the same heavy chain variable domain (VH) and light chain variable domain (VL) sequences as those of a reference anti-CD22 D1-4 antibody.

5. The engineered immune cell of claim 4, wherein:
(i) the reference anti-CD22 D1-4 antibody comprises a heavy chain CDR1 (HC-CDR1) comprising the amino acid sequence of SEQ ID NO: 67, a heavy chain CDR2 (HC-CDR2) comprising the amino acid sequence of SEQ ID NO: 68, a heavy chain CDR3 (HC-CDR3) comprising the amino acid sequence of SEQ ID NO: 69, a light chain CDR1 (LC-CDR1) comprising the amino acid sequence of SEQ ID NO: 70, a light chain CDR2 (LC-CDR2) comprising the amino acid sequence of SEQ ID NO: 71, and a light chain CDR3 (LC-CDR3) comprising the amino acid sequence of SEQ ID NO: 72; and/or
(ii) the reference anti-CD22 D1-4 antibody comprises a VH comprising the amino acid sequence of SEQ ID NO: 73 and a VL comprising the amino acid sequence of SEQ ID NO: 74.

6. The engineered immune cell of any one of claims 1-5, wherein the engineered immune cell is capable of killing a target cell that comprises on its surface both the target molecule and the recognition molecule by at least 3 fold as compared to an engineered immune cell comprising on its surface a recognition molecule comprising a binding moiety that binds to a proximal portion of the extracellular domain of the target molecule.

7. An engineered immune cell comprising on its surface a recognition molecule that comprises a binding moiety specifically binding to a target molecule on the surface of a target cell,
wherein the target molecule comprises an extracellular domain,
wherein the binding moiety specifically binds to a proximal portion of the extracellular domain,
wherein the engineered immune cell is capable of killing a target cell that comprises on its surface the target molecule, and
wherein the engineered immune cell has no or reduced capability of killing a target cell comprising on its surface both the target molecule and the recognition molecule.

8. The engineered immune cell of claim 7, wherein:
(i) the distance from the proximal portion of the extracellular domain to the membrane of the target cell is no more than about 2 times of the distance from the binding moiety to the membrane of engineered immune cell;
(ii) the extracellular domain of the target molecule is at least about 175 amino acids long, optionally wherein the binding moiety binds outside of a region that is about 80 amino acids or more away from the N-terminus of the extracellular domain; and/or optionally wherein the binding moiety binds to a region in the extracellular domain that is within about 102 amino acids from the C-terminus of the extracellular domain; and/or
(iii) the proximal portion of the extracellular domain is no more than about 90 Å away from the membrane of the target cell.

9. The engineered immune cell of claim 7 or 8, wherein the extracellular domain of the target molecule comprises two or more Ig-like domains, and wherein:
(i) the binding moiety binds to a region outside the first Ig-like domain at the N-terminal end of the extracellular domain; and/or
(ii) the binding moiety binds to a region within the first two Ig-like domains from the C-terminal end of the extracellular domain.

10. The engineered immune cell of claim 9, wherein:
(i) the binding moiety competes for binding with a reference antibody that specifically binds to an epitope within Domains 5-7 of CD22 (“anti-CD22 D5-7 antibody”);
(ii) the binding moiety binds to an epitope in Domains 5-7 of CD22 that overlaps with the binding epitope of a reference anti-CD22 D5-7 antibody;
(iii) the binding moiety comprises the same heavy chain and light chain CDR sequences as those of a reference anti-CD22 D5-7 antibody; and/or
(iv) the binding moiety comprises the same VH and VL sequences as those of a reference anti-CD22 D5-7 antibody.

11. The engineered immune cell of claim 10, wherein:
(i) the reference anti-CD22 D5-7 antibody comprises a HC-CDR1 comprising the amino acid sequence of SEQ ID NO: 76, a HC-CDR2 comprising the amino acid sequence of SEQ ID NO: 77, a HC-CDR3 comprising the amino acid sequence of SEQ ID NO: 78, a LC-CDR1 comprising the amino acid sequence of SEQ ID NO: 79, a LC-CDR2 comprising the amino acid sequence of SEQ ID NO: 80, and a LC-CDR3 comprising the amino acid sequence of SEQ ID NO: 81; and/or
(ii) the reference anti-CD22 D5-7 antibody comprises a VH comprising the amino acid sequence of SEQ ID NO: 82 and a VL comprising the amino acid sequence of SEQ ID NO: 83.

12. The engineered immune cell of any one of claims 7-11, wherein the engineered immune cell kills a target cell that comprises on its surface both the target molecule and the recognition molecule by no more than about 20% as compared to an engineered immune cell comprising on its surface a recognition molecule comprising a binding moiety that binds to a distal end of the extracellular domain of the target molecule.

13. The engineered immune cell of any one of claims 1-12, wherein the binding moiety is an sdAb, an scFv, a Fab′, a (Fab′)2, an Fv, or a peptide ligand.

14. The engineered immune cell of any one of claims 1-13, wherein the recognition molecule comprises the binding moiety, a transmembrane domain, and an intracellular signaling domain.

15. The engineered immune cell of any one of claims 1-14, wherein the target molecule is a transmembrane receptor.

16. The engineered immune cell of claim 15, wherein the target molecule is selected from the group consisting of CD22, CD4, CD21 (CR2), CD30, ROR1, CD5, and CD20.

17. The engineered immune cell of claim 16, wherein the target molecule is CD22.

18. The engineered immune cell of any one of claims 1-17, wherein the recognition molecule is multispecific.

19. The engineered immune cell of claim 18, wherein the recognition molecule comprises a second binding moiety specifically recognizing a second target molecule, and wherein the binding moiety and the second binding moiety are linked in tandem.

20. The engineered immune cell of any one of claims 14-20, wherein:
(i) the binding moiety is fused to the transmembrane domain directly or indirectly;
(ii) the binding moiety is non-covalently bound to a polypeptide comprising the transmembrane domain;
(iii) the recognition molecule comprises i) a first polypeptide comprising the binding moiety and a first member of a binding pair; and ii) a second polypeptide comprising a second member of the binding pair, wherein the first member and the second member bind to each other, and wherein the second member is fused to the transmembrane domain directly or indirectly; and/or
(iv) the binding moiety is fused to a polypeptide comprising the transmembrane domain.

21. The engineered immune cell of any one of claims 1-20, wherein the recognition molecule is a chimeric antigen receptor (“CAR”).

22. The engineered immune cell of claim 21, wherein the transmembrane domain is derived from a molecule selected from the group consisting of CD8α, CD4, CD28, 4-1BB, CD80, CD86, CD152 and PD1, optionally wherein the transmembrane domain is derived from CD8α.

23. The engineered immune cell of claim 21 or 22, wherein the intracellular signaling domain comprises a primary intracellular signaling domain derived from CD3ζ, FcRγ, FcRβ, CD3γ, CD3δ, CD3ε, CD5, CD22, CD79α, CD79b, or CD66d, optionally wherein the primary intracellular signaling domain is derived from CD3δ.

24. The engineered immune cell of any one of claims 21-23, wherein the intracellular signaling domain comprises a co-stimulatory signaling domain, optionally wherein the co-stimulatory signaling domain is derived from a co-stimulatory molecule selected from the group consisting of CD27, CD28, 4-1BB, OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, DAP10, DAP12, CD83, ligands of CD83 and combinations thereof.

25. The engineered immune cell of any one of claims 21-24, wherein the recognition molecule further comprises a hinge domain located between the C-terminus of the binding moiety and the N-terminus of the transmembrane domain, optionally wherein the hinge domain is derived from CD8α or IgG4 CH2-CH3.

26. The engineered immune cell of any one of claims 1-20, wherein the recognition molecule is a chimeric T cell receptor (“cTCR”).

27. The engineered immune cell of claim 26, wherein the transmembrane domain is derived from the transmembrane domain of a TCR subunit selected from the group consisting of TCRα, TCRβ, TCRγ, TCRδ, CD3γ, CD3ε, and CD3δ, optionally wherein the transmembrane domain is derived from the transmembrane domain of CD3ε.

28. The engineered immune cell of claim 26 or 27, wherein the intracellular signaling domain is derived from the intracellular signaling domain of a TCR subunit selected from the group consisting of TCRα, TCRβ, TCRγ, TCRδ, CD3γ, CD3ε, and CD3δ, optionally wherein the intracellular signaling domain is derived from the intracellular signaling domain of CD3ε.

29. The engineered immune cell of any one of claims 26-28, wherein the transmembrane domain and intracellular signaling domain of the recognition molecule are derived from the same TCR subunit.

30. The engineered immune cell of any one of claims 26-29, wherein the recognition molecule further comprises at least a portion of an extracellular domain of a TCR subunit.

31. The engineered immune cell of claim 30, wherein the binding moiety is fused to the N-terminus of CD3F (“eTCR”).

32. The engineered immune cell of any one of claims 1-31, wherein the engineered immune cell is a T cell.

33. The engineered immune cell of claim 32, wherein the immune cell is selected from the group consisting of a cytotoxic T cell, a helper T cell, a natural killer (NK) cell, a natural killer T (NK-T) cell, and a γδT cell.

34. The engineered immune cell of any one of claims 1-33, further comprising a co-receptor, optionally wherein the co-receptor is a chemokine receptor.

35. The engineered immune cell of any one of claims 1-34, wherein the target cell is an immune cell.

36. The engineered immune cell of any one of claims 1-34, wherein the target cell is a tumor cell.

37. A pharmaceutical composition comprising the engineered immune cell of any one of claims 1-36.

38. A method of treating an individual having a cancer, comprising administering to the individual an effective amount of the pharmaceutical composition of claim 37.

39. The method of claim 38, wherein the binding moiety specifically binds to a distal portion of the extracellular domain, and wherein the engineered immune cells are autologous to the individual.

40. The method of claim 38, wherein the binding moiety specifically binds to a proximal portion of the extracellular domain, and wherein the engineered immune cell is allogeneic to the individual.

41. The method of any one of claims 38-40, wherein the cancer is selected from the group consisting of T cell lymphoma, leukemia, B-cell precursor acute lymphoblastic leukemia (ALL), and B-cell lymphoma.

42. A method of treating an individual having an infectious disease, comprising administering to the individual an effective amount of the pharmaceutical composition of claim 37.

43. The method of claim 42, wherein the binding moiety specifically binds to a distal portion of the extracellular domain, and wherein the engineered immune cells are autologous to the individual.

44. The method of claim 42, wherein the binding moiety specifically binds to a proximal portion of the extracellular domain, and wherein the engineered immune cells are allogeneic to the individual.

45. The method of claim any one of claims 42-44, wherein the infectious disease is an infection by a virus selected from the group consisting of HIV and HTLV.

46. The method of claim 45, wherein the infectious disease is HIV.

47. A method of making the engineered immune cell of any one of claims 1-36, comprising introducing one or more nucleic acids encoding the recognition molecule into an immune cell, thereby obtaining the engineered immune cell.
